SEQUENCE LISTING (73 sequences)

SEQ ID NO: 1 - protein, 11 aa, Human immunodeficiency virus
YGRKKRRQRRR

SEQ ID NO: 2 - protein, 16 aa, Unknown
  Penetratin 1 peptide
RQIKIWFQNRRMKWKK

SEQ ID NO: 3 - DNA, 32 bp, Artificial Sequence
  Primer Xho-Oct4-F
gatcctcgagatggctggacacctggcttcag

SEQ ID NO: 4 - DNA, 33 bp, Artificial Sequence
  Primer Eco-Oct4-R
gatcgaattctcagtttgaatgcatgggagagc

SEQ ID NO: 5 - DNA, 1059 bp, Mus sp.
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactga

SEQ ID NO: 6 - DNA, 31 bp, Artificial Sequence
  Primer Xho-Sox2-F
gatcctcgagatgtataacatgatggagacg

SEQ ID NO: 7 - DNA, 33 bp, Artificial Sequence
  Primer Eco-Sox2-R
gatcgaattctcacatgtgcgacaggggcagtg

SEQ ID NO: 8 - DNA, 960 bp, Mus sp.
atgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtga

SEQ ID NO: 9 - DNA, 31 bp, Artificial Sequence
  Primer Xho-KLF4-F
gatcctcgaggctgtcagcgacgctctgctc

SEQ ID NO: 10 - DNA, 35 bp, Artificial Sequence
  Primer Eco-Klf4-R
gatcgaattcttaaaagtgcctcttcatgtgtaag

SEQ ID NO: 11 - DNA, 1452 bp, Mus sp.
atgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaa

SEQ ID NO: 12 - DNA, 31 bp, Artificial Sequence
  Primer Xho-Myc-F
gatcctcgagcccctcaacgtgaacttcacc

SEQ ID NO: 13 - DNA, 34 bp, Artificial Sequence
  Primer Eco-Myc-R
gatcgaattcttatgcaccagagtttcgaagctg

SEQ ID NO: 14 - DNA, 1365 bp, Mus sp.
ctggatttcctttgggcgttggaaaccccgcagacagccacgacgatgcccctcaacgtgaacttcaccaacaggaactatgacctcgactacgactccgtacagccctatttcatctgcgacgaggaagagaatttctatcaccagcaacagcagagcgagctgcagccgcccgcgcccagtgaggatatctggaagaaattcgagctgcttcccaccccgcccctgtccccgagccgccgctccgggctctgctctccatcctatgttgcggtcgctacgtccttctccccaagggaagacgatgacggcggcggtggcaacttctccaccgccgatcagctggagatgatgaccgagttacttggaggagacatggtgaaccagagcttcatctgcgatcctgacgacgagaccttcatcaagaacatcatcatccaggactgtatgtggagcggtttctcagccgctgccaagctggtctcggagaagctggcctcctaccaggctgcgcgcaaagacagcaccagcctgagccccgcccgcgggcacagcgtctgctccacctccagcctgtacctgcaggacctcaccgccgccgcgtccgagtgcattgacccctcagtggtctttccctacccgctcaacgacagcagctcgcccaaatcctgtacctcgtccgattccacggccttctctccttcctcggactcgctgctgtcctccgagtcctccccacgggccagccctgagcccctagtgctgcatgaggagacaccgcccaccaccagcagcgactctgaagaagagcaagaagatgaggaagaaattgatgtggtgtctgtggagaagaggcaaacccctgccaagaggtcggagtcgggctcatctccatcccgaggccacagcaaacctccgcacagcccactggtcctcaagaggtgccacgtctccactcaccagcacaactacgccgcacccccctccacaaggaaggactatccagctgccaagagggccaagttggacagtggcagggtcctgaagcagatcagcaacaaccgcaagtgctccagccccaggtcctcagacacggaggaaaacgacaagaggcggacacacaacgtcttggaacgtcagaggaggaacgagctgaagcgcagcttttttgccctgcgtgaccagatccctgaattggaaaacaacgaaaaggcccccaaggtagtgatcctcaaaaaagccaccgcctacatcctgtccattcaagcagacgagcacaagctcacctctgaaaaggacttattgaggaaacgacgagaacagttgaaacacaaactcgaacagcttcgaaactctggtgcataa

SEQ ID NO: 15 - DNA, 51 bp, Artificial Sequence
  Primer Sall4-F
cagcgccgccgcggtggatccaccatggccatgtcgaggcgcaagcaggcg

SEQ ID NO: 16 - DNA, 50 bp, Artificial Sequence
  Primer Sall4-R
aagcttcgaattcaccgcatgcacttagctgacagcaatcttattttcct

SEQ ID NO: 17 - DNA, 3204 bp, Mus sp.
atgtcgaggcgcaagcaggcgaagccccagcacatcaactgggaggagggccagggcgagcagcctcagcagctaccgagccccgacctcgccgaggcgctggcggcggaggaacccggtgctccagtgaactcccctgggaactgcgatgaagcctcagaggactccataccggtgaagcggccccggcgggaggacactcacatctgcaacaaatgctgtgccgagttctttagtctctctgaattcatggaacacaagaaaagttgcactaaaacccctcctgtcctcatcatgaatgacagcgaggggccagtgccttcagaggacttttccagagctgccctgagccaccagctgggcagcccaagcaataaagacagtctccaggagaacggcagcagctcgggggacttgaagaagctgggcacggactccatcctgtacttgaagacagaggctacccagccatccacaccccaggacataagctatttacccaaaggcaaagtagccaacaccaatgtgactctgcaggcgctccgcggcaccaaggtggccgtgaaccaacggggtgcagaggcacccatggcgcccatgcctgctgcccaaggcatcccttgggtcctggagcagatcctgtgcctgcagcagcagcaactccagcaaatccagcttacggaacagattcgcgtccaggtgaacatgtgggcagcgcacgcgctccactctggagtggcgggggccgacacgctgaaggccttaagcagccatgtgtctcagcaagtgtccgtgtcccagcaggtgtcggctgccgtggccctgctcagccagaaagcctcaaacccagctctgtcgctcgatgccttgaaacaagccaagctacctcatgccagcgtcccctccgcagccagcccgttgtcctcggggttaacgtccttcaccttgaagcctgacgggacacgggttctccccaacttcgtgtctcgccttcccagtgccctgctacctcagactccgggctctgtgctcctgcagagtcccttctccgctgtgacgctcgaccagtccaagaaaggaaaggggaaaccccagaacctctccgcctctgcctcggtgttagatgtcaaggccaaggacgaagtcgtcctcggtaagcacaagtgtaggtactgtcccaaggttttcgggacagatagctcccttcagattcaccttcgctcccacaccggagagagaccttacgtgtgccctatctgtggtcaccgcttcaccaccaagggcaatctcaaggtccacttacaacgacaccctgaggtgaaggcaaacccccagctgttggccgaattccaggacaaaggggcagtgagtgccgcttctcactatgcactccctgtccccgtccctgccgatgaatcgagtctctctgtagacgccgagcctgtcccggtcacgggaaccccttctctagggctacctcaaaagctcacgtcagggcctaattccagggacctcatgggtggctccttgcccaatgacatgcagccagggccttctccagaaagtgaggcgggccttccactccttggggtggggatgatacataatcccccaaaggctgggggcttccagggcactggggccccagagtcagggtccgagaccctgaaattgcagcaactagtggagaacatagacaaggccactactgaccccaacgagtgtctcatttgtcatcgggtcctcagctgtcagagttccctgaagatgcattaccgtacccacacaggggagagaccattccagtgcaagatctgtggccgggccttctccaccaaaggcaacctgaagacacaccttggggttcaccgaaccaacacgaccgtaaagacccaacattcgtgccccatctgccagaagaaattcaccaacgccgtcatgttacagcagcatatccggatgcacatgggtggccagatccccaacacccctctgccagagagtccctgtgacttcacggctcccgagcccgtggccgtcagtgagaatggcagtgccagcggggtctgccaggacgacgcagcagaagggatggaagccgaggaggtctgttctcaggatgttcccagtggcccctcaactgtctctctgccggttcccagtgcccacctggcatcgccctctctgggcttctctgtgttggcctccctggatacgcaggggaaaggggctcttccggcgctggccctgcagaggcagagcagtcgagaaaacagctccctggagggcggtgacactggtccagccaatgactcttccttgctcgtgggtgaccaggagtgtcagagccgaagcccagatgccacggagaccatgtgctaccaggcagtgtcacctgccaatagccaagccggaagtgtcaagtcccggtctcccgagggtcacaaggccgagggcgtggagagctgccgcgttgacaccgaaggtcgtaccagcctccctccaacatttatccgagcacagcccacctttgtcaaagttgaagtgcctggcacctttgtgggaccccccagcatgccctcgggtatgccgcctttgctagcatcgcagccgcagccacgccgccaggccaagcagcactgctgcacacggtgtggaaagaacttctcgtctgccagtgccctgcagatccacgagcgaacacacacgggagagaagcctttcgtgtgtaacatatgcgggcgggccttcaccacgaaaggcaacctgaaggtacactacatgactcatggggccaacaataactccgcccgccggggaaggaagctggccatagagaaccccatggccgcgctgagtgctgagggaaagagagcgcccgaggtgttttccaaggagctcctgtcccccgcggtgagtgtggaccccgcctcctggaaccagtacaccagcgtcctgaatgggggtctggccatgaagaccaacgagatctccgtgatccagagcggaggcatccccacgctgcctgtgtcgctgggggccagctctgtggtgagcaatggcacgatttccaagcttgacggctctcagaccggtgtgagcatgcccatgagcgggaacggagaaaagctcgctgttcccgacggcatggccaaacaccagttccctcacttcctggaggaaaataagattgctgtcagctaa

SEQ ID NO: 18 - DNA, 34 bp, Artificial Sequence
  Primer Xho-EGFP-F
gatcctcgagatggtgagcaagggcgaggagctg

SEQ ID NO: 19 - DNA, 41 bp, Artificial Sequence
  Primer Eco-EGFP-R
gatcgaattctcagttatctacttgtacagctcgtccatgc

SEQ ID NO: 20 - DNA, 728 bp, Unknown
  EGFP sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtagataactga

SEQ ID NO: 21 - DNA, 37 bp, Artificial Sequence
  Primer Xho-hsMyc-F
gatcctcgagatgcccctcaacgttagcttcaccaac

SEQ ID NO: 22 - DNA, 34 bp, Artificial Sequence
  Primer Eco-hsMyc-R
gatcgaattcttacgcacaagagttccgtagctg

SEQ ID NO: 23 - DNA, 1365 bp, Homo sapiens
ctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaa

SEQ ID NO: 24 - DNA, 33 bp, Artificial Sequence
  Primer Nco-hsOct4-F
gatcccatggcgggacacctggcttcggatttc

SEQ ID NO: 25 - DNA, 33 bp, Artificial Sequence; identical to SEQ ID NO: 4
  Eco-hsOct4-R

SEQ ID NO: 26 - DNA, 1083 bp, Homo sapiens
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactga

SEQ ID NO: 27 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 28 - DNA, 32 bp, Artificial Sequence
  Primer Xho-hsSox2-F
gatcctcgagatgtacaacatgatggagacgg

SEQ ID NO: 29 - DNA, 34 bp, Artificial Sequence
  Primer Eco-hsSox2-R
gatcgaattctcacatgtgtgagaggggcagtgt

SEQ ID NO: 30 - DNA, 954 bp, Homo sapiens
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtga

SEQ ID NO: 31 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 32 - DNA, 33 bp, Artificial Sequence
  Primer Xho-hsKlf4-F
gatcctcgagatggctgtcagcgacgcgctgct

SEQ ID NO: 33 - DNA, 36 bp, Artificial Sequence
  Primer Eco-hsKlf4-R
gatcgaattcttaaaaatgcctcttcatgtgtaagg

SEQ ID NO: 34 - DNA, 1440 bp, Homo sapiens
atgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 35 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 36 - DNA, 35 bp, Artificial Sequence
  Primer Kpn-hsSall4-F
gatcggtaccatgtcgaggcgcaagcaggcgaaac

SEQ ID NO: 37 - DNA, 34 bp, Artificial Sequence
  Primer Eco-hsSall4-R
gatcgaattcttagctgaccgcaatcttgttttc

SEQ ID NO: 38 - DNA, 3162 bp, Homo sapiens
atgtcgaggcgcaagcaggcgaaaccccagcacatcaactcggaggaggaccagggcgagcagcagccgcagcagcagaccccggagtttgcagatgcggccccagcggcgcccgcggcgggggagctgggtgctccagtgaaccacccagggaatgacgaggtggcgagtgaggatgaagccacagtaaagcggcttcgtcgggaggagacgcacgtctgtgagaaatgctgtgcggagttcttcagcatctctgagttcctggaacataagaaaaattgcactaaaaatccacctgtcctcatcatgaatgacagcgaggggcctgtgccttcagaagacttctccggagctgtactgagccaccagcccaccagtcccggcagtaaggactgtcacagggagaatggcggcagctcagaggacatgaaggagaagccggatgcggagtctgtggtgtacctaaagacagagacagccctgccacccaccccccaggacataagctatttagccaaaggcaaagtggccaacactaatgtgaccttgcaggcactacggggcaccaaggtggcggtgaatcagcggagcgcggatgcactccctgcccccgtgcctggtgccaacagcatcccgtgggtcctcgagcagatcttgtgtctgcagcagcagcagctacagcagatccagctcaccgagcagatccgcatccaggtgaacatgtgggcctcccacgccctccactcaagcggggcaggggccgacactctgaagaccttgggcagccacatgtctcagcaggtttctgcagctgtggctttgctcagccagaaagctggaagccaaggtctgtctctggatgccttgaaacaagccaagctacctcacgccaacatcccttctgccaccagctccctgtccccagggctggcacccttcactctgaagccggatgggacccgggtgctcccgaacgtcatgtcccgcctcccgagcgctttgcttcctcaggccccgggctcggtgctcttccagagccctttctccactgtggcgctagacacatccaagaaagggaaggggaagccaccgaacatctccgcggtggatgtcaaacccaaagacgaggcggccctctacaagcacaagtgtaagtactgtagcaaggtttttgggactgatagctccttgcagatccacctccgctcccacactggagagagacccttcgtgtgctctgtctgtggtcatcgcttcaccaccaagggcaacctcaaggtgcactttcaccgacatccccaggtgaaggcaaacccccagctgtttgccgagttccaggacaaagtggcggccggcaatggcatcccctatgcactctctgtacctgaccccatagatgaaccgagtctttctttagacagcaaacctgtccttgtaaccacctctgtagggctacctcagaatctttcttcggggactaatcccaaggacctcacgggtggctccttgcccggtgacctgcagcctgggccttctccagaaagtgagggtggacccacactccctggggtgggaccaaactataattccccaagggctggtggcttccaagggagtgggacccctgagccagggtcagagaccctgaaattgcagcagttggtggagaacattgacaaggccaccactgatcccaacgaatgtctcatttgccaccgagtcttaagctgtcagagctccctcaagatgcattatcgcacccacaccggggagagaccgttccagtgtaagatctgtggccgagccttttctaccaaaggtaacctgaagacacaccttggggttcaccgaaccaacacatccattaagacgcagcattcgtgccccatctgccagaagaagttcactaatgccgtgatgctgcagcaacatattcggatgcacatgggcggtcagattcccaacacgcccctgccagagaatccctgtgactttacgggttctgagccaatgaccgtgggtgagaacggcagcaccggcgctatctgccatgatgatgtcatcgaaagcatcgatgtagaggaagtcagctcccaggaggctcccagcagctcctccaaggtccccacgcctcttcccagcatccactcggcatcacccacgctagggtttgccatgatggcttccttagatgccccagggaaagtgggtcctgccccttttaacctgcagcgccagggcagcagagaaaacggttccgtggagagcgatggcttgaccaacgactcatcctcgctgatgggagaccaggagtatcagagccgaagcccagatatcctggaaaccacatccttccaggcactctccccggccaatagtcaagccgaaagcatcaagtcaaagtctcccgatgctgggagcaaagcagagagctccgagaacagccgcactgagatggaaggtcggagcagtctcccttccacgtttatccgagccccgccgacctatgtcaaggttgaagttcctggcacatttgtgggaccctcgacattgtccccagggatgacccctttgttagcagcccagccacgccgacaggccaagcaacatggctgcacacggtgtgggaagaacttctcgtctgctagcgctcttcagatccacgagcggactcacactggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcagctaa

SEQ ID NO: 39 - DNA, 1851 bp, Homo sapiens
  misc_feature (337)..(337): n is a, c, g, or t
atgtcgaggcgcaagcaggcgaaaccccagcacatcaactcggaggaggaccagggcgagcagcagccgcagcagcagaccccggagtttgcagatgcggccccagcggcgcccgcggcgggggagctgggtgctccagtgaaccacccagggaatgacgaggtggcgagtgaggatgaagccacagtaaagcggcttcgtcgggaggagacgcacgtctgtgagaaatgctgtgcggagttcttcagcatctctgagttcctggaacataagaaaaattgcactaaaaatccacctgtcctcatcatgaatgacagcgaggggcctgtgccttcanaagacttctccggagctgtactgagccaccagcccaccagtcccggcagtgaggactgtcacagggagaatggcggcagctcanaggacataaaggagaagccggatgcggagtctgtggtgtacctaaagacagagacagccctgccacccaccccccaggacataagctatttagccaaaggcaaagtggccaacactaacgtgaccttgcaggcactacggggcaccaaggtggcggtgaatcagcggagcgcggatgcactccctgcccccgtgcctggtgccaacagcatcccgtgggtcctcgagcagatcttgtgtctgcagcagcagcagctacagcagatccagctcaccgagcagatccgcatccaggtgaacatgtgggcctcccacgccctccactcaagcggggcaggggccgacactctgaagaccttgggcagccacatgtctcagcaggtttctgcagctgtggctttgctcagccagaaagctggaagccaaggtctgtctctggatgccttgaaacaagccaagctacctcacgccaacatcccttctgccaccagctccctgtccccagggctggcacccttcactctgaagccggatgggacccgggtgctcccgaacgtcatgtcccgcctcccgagcgctttgcttcctcaggccccgggctcggtgctcttccagagccctttctccactgtggcgctagacacatccaagaaagggaaggggaagccaccgaacatctccgcggtggatgtcaaacccaaagacgaggcggccctctacaagcacaagtgtcggagcagtctcccttccacgtttatccgagccccgccgacctatgtcaaggttgaagttcctggcacatttgtgggaccctcgacattgtccccagggatgacccctttgttagcagcccagccacgcggacaggccaagcaacatggctgcacacggtgtggnaagaacttntcgtntgntagcgctcttcagatccacgagcggactcacantggagagaagccttttgtgtgcaacatttgtgggcgagcttttaccaccaaaggcaacttaaaggttcactacatgacacacggggcgaacaataactcagcccgccgtggaaggaagttggccatcgagaacaccatggctctgttaggtacggacggaaaaagagtctcagaaatctttcccaaggaaatcctggccccttcagtgaatgtggaccctgttgtgtggaaccagtacaccagcatgctcaatggcggtctggccgtgaagaccaatgagatctctgtgatccagagtgggggggttcctaccctcccggtttccttgggggccacctccgttgtgaataacgccactgtctccaagatggatggctcccagtcgggtatcagtgcagatgtggaaaaaccaagtgctactgacggcgttcccaaacnccagtttcctcacttcctggaagaaaacaagantgcggtcagctaa

SEQ ID NO: 40 - DNA, 35 bp, Artificial Sequence
  Primer h-UTf1-F
gatcctcgagatgctgctccggccccgcaggccgc

SEQ ID NO: 41 - DNA, 36 bp, Artificial Sequence
  Primer h-UTF1-R
gatcgaattctcactggcacgggtccctgaggaccc

SEQ ID NO: 42 - DNA, 1026 bp, Homo sapiens
atgctgctccggccccgcaggccgcccccgctcgcgccccccgcgccgccctcgcccgccagccccgaccccgagccgcggacacccggagacgccccggggaccccgccccggaggcccgcctcgcccagcgcgctgggggaactcgggttgccggtgtccccgggctcggcgcagcgcacgccctggagcgcccgggagacggagctgctgctggggacgctgctgcaaccggccgtgtggcgcgcgctgctcctggaccgccgccaggccctgcccacctaccgccgcgtgtcggccgcgctggcccagcagcaggtgcgccgcacccccgcgcagtgccgccgccgctacaagttccttaaagacaagtttcgcgaggcgcacggccagccgcccgggcccttcgacgagcagatccggaagctcatggggctgctgggcgacaacgggcgcaaacggcctcgccgccgctccccggggtccgggcgcccccagcgcgcccgccgcccggtccccaacgcgcacgcgccggctcccagcgaaccagacgccaccccgctgcccaccgcccgcgaccgcgacgcggaccccacctggacgctccgcttcagcccgtccccaccgaagtctgcggacgcctcccccgcccccggctccccgccagctcccgccccgaccgccctcgccacctgcatccccgaggaccgcgcgcccgtccgcggccccgggtccccgccgccacccccggcccgcgaagaccccgactcgccgcccggccgccccgaggactgcgcgccccctccggccgcgcccccgtcgctgaacaccgccctgctgcagaccctggggcacctgggcgacatcgcgaacatcctgggcccgctgcgcgaccagctgctgaccttgaaccagcacgtggagcagctgcgcggcgccttcgaccagacagtgtccctggccgtgggcttcattctgggcagcgcggccgccgagcgaggggtcctcagggacccgtgccagtga

SEQ ID NO: 43 - DNA, 1337 bp, Artificial Sequence
  Mouse myc codon optimized sequence
gatcctcgagccactcaacgttaattttaccaatcgtaattatgacctcgactatgactcagtccagccttacttcatctgtgatgaggaagaaaacttctaccaccaacagcagcaaagcgaactgcaaccgcccgcgcctagtgaagatatttggaaaaaatttgaattactgccgaccccccccctgtccccgtcccgtcgttcaggactttgtagcccgtcttatgtggccgtcgcgactagcttttcacctcgtgaggacgatgatggaggcggtggcaacttttcgaccgcagatcaactcgaaatgatgacagaacttttaggcggagatatggtaaatcagtctttcatttgtgaccctgatgacgaaacctttatcaaaaacattattattcaagattgcatgtggtctggctttagcgccgccgcgaaacttgtaagcgaaaaattagcctcatatcaagcagcacgcaaagattctacctcactcagccctgcccgcggacactctgtatgttccacgtcttctctgtacctccaagaccttactgccgcagccagcgaatgtattgacccgagtgttgtgtttccatatccactgaatgattcctctagtcccaaatcttgtacctcatccgacagcaccgcattctcgccgagctcagactcactgttatcctccgaaagcagccctcgcgcctcccccgaaccattggttttacacgaagaaacaccaccaaccacttcatccgactctgaagaagaacaagaagacgaagaagaaattgatgtagtcagtgtggaaaagcgtcaaaccccggcgaaacgtagcgaatctggttcctctccctcgcgcggacattctaaacccccacatagccccctcgttttaaaacgttgtcacgtttcaactcaccagcataattatgcagcaccaccatctacccgcaaagactatccagcagcaaaacgcgccaaactcgattccggccgcgtcctgaagcaaatttctaacaatcgcaaatgttcctcaccccgttcatccgataccgaagaaaatgataaacgccgtacccataacgttctggaacgccaacgccgtaacgaactgaaacgttcctttttcgcattgcgcgatcagatcccggagctcgaaaataatgaaaaagcacctaaagtagttatcctgaaaaaagcaaccgcatatattctgagcattcaagccgacgaacacaaattaacatccgaaaaagacttattacgtaaacgtcgcgaacaactgaaacataaactggaacaattacgcaactccggagcgtaagaattcgatc

SEQ ID NO: 44 - protein, 438 aa, Artificial Sequence
  Mouse myc codon optimized sequence
PLNVNFTNRNYDLDYDSVQPYFICDEEENFYHQQQQSELQPPAPSEDIWKKFELLPTPPLSPSRRSGLCSPSYVAVATSFSPREDDDGGGGNFSTADQLEMMTELLGGDMVNQSFICDPDDETFIKNIIIQDCMWSGFSAAAKLVSEKLASYQAARKDSTSLSPARGHSVCSTSSLYLQDLTAAASECIDPSVVFPYPLNDSSSPKSCTSSDSTAFSPSSDSLLSSESSPRASPEPLVLHEETPPTTSSDSEEEQEDEEEIDVVSVEKRQTPAKRSESGSSPSRGHSKPPHSPLVLKRCHVSTHQHNYAAPPSTRKDYPAAKRAKLDSGRVLKQISNNRKCSSPRSSDTEENDKRRTHNVLERQRRNELKRSFFALRDQIPELENNEKAPKVVILKKATAYILSIQADEHKLTSEKDLLRKRREQLKHKLEQLRNSGA

SEQ ID NO: 45 - DNA, 1340 bp, Artificial Sequence
  Human myc codon optimized DNA sequence
gatcctcgagatgccccttaatgtctcatttacgaaccgtaactacgatcttgattacgacagcgttcaaccttacttttactgcgatgaagaagaaaatttctatcagcaacaacagcaaagcgaactgcaacccccggccccttcagaggatatctggaaaaaattcgaacttttgccaaccccgcccctgtcaccttctcgccgctctggtttatgctccccgtcctatgtagccgtcactccattttccttacgtggtgataacgacggtggtggcggtagcttttcaaccgccgatcagttagaaatggttaccgaactcttaggcggcgatatggttaatcagtctttcatttgtgacccagatgacgaaacctttattaaaaacattatcattcaagactgcatgtggtctggtttctcagccgccgcaaaacttgtgtctgaaaaacttgcatcctaccaagctgcccgcaaagattccggctccccaaaccccgctcgtggccattccgtgtgtagcacctcgtccctttatttgcaggacttatcagcagcagcatctgaatgtatcgatccgtccgttgtcttcccatacccgttgaatgactcaagctctccaaaatcctgcgcctcccaagattcctccgcttttagcccctcctccgatagtctcctttcttccaccgagagttccccacagggatccccagaaccgttagttttgcacgaagaaacgcctccaaccacctcaagcgatagcgaagaagaacaagaagatgaagaagaaattgatgttgtttccgttgaaaaacgccaagccccaggtaaacgctccgaatccggctctccatccgctggcggccactctaaaccacctcatagcccgttagtactcaaacgctgccatgtctctacccatcaacataattatgccgcacctccaagtacgcgcaaagactacccagcagccaaacgcgtgaaactggatagtgtccgtgtcctccgtcaaattagcaataatcgtaaatgcacttctccccggtcctcagatactgaagaaaacgtaaaacgccgtactcataacgtcttagaacgtcagcgccgtaacgaactgaaacgctcattttttgcgcttcgtgatcaaatccccgaattagaaaataatgaaaaagcgcctaaagttgttatcctgaaaaaagccacagcctatatcttatccgtacaagccgaagaacaaaaacttatctctgaagaagatctgctccgcaaacgccgtgaacaattaaaacataaactggaacaattacgtaatagctgcgcctaagaattcgatc

SEQ ID NO: 46 - protein, 439 aa, Artificial Sequence
  Human myc codon optimized DNA sequence
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 47 - DNA, 1103 bp, Artificial Sequence
  Human oct4 codon optimized DNA sequence
gatcccatggatggctggtcatcttgcaagtgatttcgccttttcacctcccccaggtggcggcggtgacggtccgggcggtccagaaccaggttgggttgatccacgcacgtggttaagttttcaaggtcctccaggtggtccaggaattggtcccggtgttggccccggcagtgaagtgtggggcatccccccgtgtcctcccccctatgaattttgcggtggcatggcgtattgcggtcctcaagttggtgttggtttggtcccacaaggtggtctcgaaacctcacaacccgaaggagaagctggcgtgggtgtagaatcaaacagcgatggcgcctcacctgaaccatgcactgtcactcctggcgcggttaaattggaaaaagaaaaattagagcagaacccagaagaatcccaagatatcaaagcccttcagaaagaattagaacaatttgccaaactcttgaaacaaaaacgtatcactctcggatatacgcaagccgatgttggcctgaccctcggtgtattattcgggaaagtattttcacagacaacaatctgccgttttgaagcactgcaactgtcttttaaaaacatgtgcaaattacgccccctgctgcagaaatgggtcgaagaagcagataacaatgaaaacttacaggaaatttgcaaggccgaaaccttagttcaagctcgcaaacgtaaacgcaccagcattgaaaatcgtgtacgtggtaatctcgaaaatttattcttacagtgtcctaaaccaactttacagcaaatcagccatatcgctcagcaactcggtcttgagaaagacgtcgttcgggtttggttttgtaatcgtcgtcaaaaaggtaaacgctcgtcatccgactacgcccaacgggaagattttgaagctgcaggtagtccctttagtggcggccccgtttcgttccccctcgctccaggcccacattttggtaccccaggttacggtagtcctcattttacagcattatattcatccgttccgtttcccgaaggcgaggcattccctccagtatcggttactactctcggctcacctatgcactccaattaagaattcgatc

SEQ ID NO: 48 - protein, 360 aa, Artificial Sequence; identical to SEQ ID NO: 27
  Human Oct4 codon optimized protein sequence

SEQ ID NO: 49 - DNA, 974 bp, Artificial Sequence
  Human Sox2 codon optimized DNA sequence
gatcctcgagatgtacaacatgatggaaacagaactcaaacctccaggccctcaacaaacttccggtggtggcggcggcaactcaactgcagcagcagcaggtggtaatcagaaaaatagcccggatcgtgttaaacgcccgatgaacgcatttatggtatggtcccgcggtcaacgtcggaaaatggctcaagaaaaccctaaaatgcataacagcgaaatttctaaacgtttaggtgctgaatggaaactcttatctgaaaccgaaaaacgtccgtttattgatgaagccaaacgcttgcgcgcgctccacatgaaagaacatcccgattataaataccgtcctcgtcgtaaaaccaaaacgttaatgaaaaaagataaatacactcttccaggtggtctcttagctccaggcggtaactctatggcgtcaggggtcggggtcggtgctggactgggggccggagttaatcagcgtatggactcttatgcccacatgaacggttggtcaaatggcagctacagcatgatgcaagatcagcttggttatcctcaacatcccggtttgaacgctcatggcgcagctcaaatgcaaccgatgcaccgttacgacgtatccgcattacagtataacagtatgactagctcgcaaacttacatgaatggatcaccgacctacagtatgagttattcacaacaaggcacccccggcatggccttaggctcaatgggctccgtcgtcaaatccgaagcatcctcttccccaccagtcgttacgtcctcctcacactctcgtgcaccttgtcaagctggagatttacgcgatatgatctcaatgtatctccccggcgcagaagtaccagaaccagccgctccttcacgtcttcacatgtctcagcattatcaatctggccctgttccaggtaccgcaattaacggcacattaccattatctcacatgtaagaattcgatc

SEQ ID NO: 50 - protein, 317 aa, Artificial Sequence; identical to SEQ ID NO: 31
  Human Sox2 codon optimized protein sequence

SEQ ID NO: 51 - DNA, 1433 bp, Artificial Sequence
  Human klf4 codon optimized DNA sequence
gatcctcgagatggccgtctccgacgcactgttgcctagcttcagcacctttgcttcaggtcccgcaggccgcgaaaaaacactccgccaagcaggcgcccccaataaccgttggcgcgaagaactttcacatatgaaacgtctgcccccagtgttgccgggtcgcccttatgatttagctgcagcgaccgtggccaccgacctcgaatcgggtggagcaggcgcagcctgtggtggcagtaatttagcccctcttccccgtcgcgaaactgaagaatttaatgatctgttagacctcgactttattttatccaactctctcacccatccaccagaatcagtcgccgcaactgtttcgtcctccgcatcagcttcatcgtctagctctccgtcgtcaagcggccctgcatcggccccatctacatgctcttttacataccccatccgcgctggtaacgatccgggtgttgccccaggaggtaccggaggaggcttactgtatggtcgcgaatcagcccctccaccgacagccccgttcaaccttgccgatattaatgacgtgtcccctagtggtggctttgtggccgaattgctgcgtccagaacttgaccccgtttatatcccgcctcaacaacctcagccccctggcggtggcctcatgggtaaatttgtcttaaaagcaagcttgtccgcacctggttccgaatatggtagtccttccgttatctctgtttccaagggttctcctgatggctcccatccagttgtagttgcaccttataatggcggtcccccacgtacctgtcctaaaatcaaacaggaagctgtttcctcctgcacacatttaggtgccggccctcctctgagcaacggccatcgcccagcggcccacgatttccctttaggtcgtcaacttccatcccgtacgacaccaaccttaggcttagaagaagtcctgtcctctcgtgactgccatcctgctttacctctgcctccaggttttcatccacatccaggcccgaattacccttccttcttaccagatcaaatgcaaccacaagtcccccccttacactaccaagaactgatgccaccgggctcctgcatgccagaagaaccaaaaccgaaacgcggccgccgttcctggccccgcaaacgtaccgccacccacacctgtgactatgctggttgcggcaaaacatacactaaaagttcacaccttaaagcacatcttcgtacgcatactggcgaaaaaccttatcactgcgattgggatggctgtggttggaaattcgcacgctccgatgagttaacccgtcattatcgcaaacatactggacatcgcccattccaatgccaaaaatgcgatcgcgcgttttcccgttcagaccatttagccttacacatgaaacgccacttttaagaattcgatc

SEQ ID NO: 52 - protein, 470 aa, Artificial Sequence; identical to SEQ ID NO: 35
  Human klf4 codon optimized protein sequence

SEQ ID NO: 53 - DNA, 36 bp, Artificial Sequence
  Primer Age/xhoSense
accggtccgcctaaaaagaaacgcaaagtactcgag

SEQ ID NO: 54 - DNA, 36 bp, Artificial Sequence
  Primer Age/XhoRev-comp
ctcgagtactttgcgtttctttttaggcggaccggt

SEQ ID NO: 55 - DNA, 30 bp, Artificial Sequence
  Primer Nls-sen (Age/Xho)
ccggtccgcctaaaaagaaacgcaaagtac

SEQ ID NO: 56 - DNA, 30 bp, Artificial Sequence
  Primer Nls-anti(age/Xho)
tcgagtactttgcgtttctttttaggcgga

SEQ ID NO: 57 - DNA, 27 bp, Artificial Sequence
  Primer Nls-sen (age/xho)-P
ccggtcctaaaaagaaacgcaaagtac

SEQ ID NO: 58 - DNA, 27 bp, Artificial Sequence
  Primer Nls-anti(age/Xho)-P
tcgagtactttgcgtttctttttagga

SEQ ID NO: 59 - DNA, 39 bp, Artificial Sequence
  Primer Nco1NLS sense
ccatggccccgcctaaaaagaaacgcaaagtagccatgg

SEQ ID NO: 60 - DNA, 39 bp, Artificial Sequence
  Primer Nco1NLS antisense
ccatggctactttgcgtttctttttaggcggggccatgg

SEQ ID NO: 61 - DNA, 33 bp, Artificial Sequence
  Primer Nco1NLS sense
catggccccgcctaaaaagaaacgcaaagtagc

SEQ ID NO: 62 - DNA, 33 bp, Artificial Sequence
  Primer Nco1NLS antisense
catggctactttgcgtttctttttaggcggggc

SEQ ID NO: 63 - DNA, 29 bp, Artificial Sequence
  Primer Nco1NLS sense-G-P
catggcccctaaaaagaaacgcaaagtac

SEQ ID NO: 64 - DNA, 29 bp, Artificial Sequence
  Primer Nco1NLS antisense-G-P
catggtactttgcgtttctttttaggggc

SEQ ID NO: 65 - DNA, 36 bp, Artificial Sequence
  Primer HDM2-F
gatcctcgagatgtgcaataccaacatgtctgtacc

SEQ ID NO: 66 - DNA, 35 bp, Artificial Sequence
  Primer HDM2-R
gatcgaattcctaggggaaataagttagcacaatc

SEQ ID NO: 67 - DNA, 1476 bp, Unknown
  HDM2 cloned sequence
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctag

SEQ ID NO: 68 - DNA, 33 bp, Artificial Sequence
  Primer p53-F
gatcctcgagatggaggagccgcagtcagatcc

SEQ ID NO: 69 - DNA, 35 bp, Artificial Sequence
  Primer p53-393R
gatcgaattctcagtctgagtcaggcccttctgtc

SEQ ID NO: 70 - DNA, 36 bp, Artificial Sequence
  Primer Mdm2-F
gatcctcgagatgtgcaataccaacatgtctgtgtc

SEQ ID NO: 71 - DNA, 35 bp, Artificial Sequence
  Primer Mdm2-R
gatcgaattcctagttgaagtaacttagcacaatc

SEQ ID NO: 72 - DNA, 1470 bp, Mus sp.
atgtgcaataccaacatgtctgtgtctaccgagggtgctgcaagcacctcacagattccagcttcggaacaagagactctggttagaccaaaaccattgcttttgaagttgttaaagtccgttggagcgcaaaacgacacttacactatgaaagagattatattttatattggccagtatattatgactaagaggttatatgacgagaagcagcagcacattgtgtattgttcaaatgatctcctaggagatgtgtttggagtcccgagtttctctgtgaaggagcacaggaaaatatatgcaatgatctacagaaatttagtggctgtaagtcagcaagactctggcacatcgctgagtgagagcagacgtcagcctgaaggtgggagtgatctgaaggatcctttgcaagcgccaccagaagagaaaccttcatcttctgatttaatttctagactgtctacctcatctagaaggagatccattagtgagacagaagagaacacagatgagctacctggggagcggcaccggaagcgccgcaggtccctgtcctttgatccgagcctgggtctgtgtgagctgagggagatgtgcagcggcggcacgagcagcagtagcagcagcagcagcgagtccacagagacgccctcgcatcaggatcttgacgatggcgtaagtgagcattctggtgattgcctggatcaggattcagtttctgatcagtttagcgtggaatttgaagttgagtctctggactcggaagattacagcctgagtgacgaagggcacgagctctcagatgaggatgatgaggtctatcgggtcacagtctatcagacaggagaaagcgatacagactcttttgaaggagatcctgagatttccttagctgactattggaagtgtacctcatgcaatgaaatgaatcctccccttccatcacactgcaaaagatgctggacccttcgtgagaactggcttccagacgataaggggaaagataaagtggaaatctctgaaaaagccaaactggaaaactcagctcaggcagaagaaggcttggatgtgcctgatggcaaaaagctgacagagaatgatgctaaagagccatgtgctgaggaggacagcgaggagaaggccgaacagacgcccctgtcccaggagagtgacgactattcccaaccatcgacttccagcagcattgtttatagcagccaagaaagcgtgaaagagttgaaggaggaaacgcagcacaaagacgagagtgtggaatctagcttctccctgaatgccatcgaaccatgtgtgatctgccaggggcggcctaaaaatggctgcattgttcacggcaagactggacacctcatgtcatgtttcacgtgtgcaaagaagctaaaaaaaagaaacaagccctgcccagtgtgcagacagccaatccaaatgattgtgctaagttacttcaactag

SEQ ID NO: 73 - protein, 8 aa, Unknown
  Nuclear translocation peptide
PPKKKRKV

(19) United States
(12) Patent Application Publication
(10) Pub. No.: US 2012/0115225 A1
(43) Pub. Date: May 10, 2012

(54) REPROGRAMMING OF SOMATIC CELLS WITH PURIFIED PROTEINS

(76) Inventor: C. W. Xu, Pasadena, CA (US)

(21) Appl. No.: 13/265,006

(22) PCT Filed: Apr. 22, 2010
(86) PCT No.: PCT/US10/32131

§ 371 Date: Jan. 9, 2012

Related U.S. Application Data
(60) Provisional application No. 61/172,168, filed on Apr. 23, 2009.

(51) Int. Cl. C12N 5/071; C12N 5/0775

(52) U.S. Cl. 435/366; 435/377

(57) ABSTRACT

Purified somatic cell reprogramming factors are described herein. The factors are particularly useful alone or in combination with at least one effector of cellular metabolism, in order to generate at least one reprogramming somatic cell. Methods for using at least one somatic cell reprogramming factor and at least one somatic cell reprogramming enhancing factor are pro-vided. Additionally, the cells generated from the methods are also described. The methods and cells may find use in personalized medicine applications.

### CROSS REFERENCE TO PRIOR U.S. APPLICATION

This application claims benefit under 35 U.S.C. §119(e) to U.S. Provisional Application Ser. No. 61/172,168, filed Apr. 23, 2009, hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: NVCI_{—}009_{—}01WO_SeqList_ST25.txt, date recorded: Apr. 21, 2010, file size 76 kilobytes).

### FIELD OF THE INVENTION

The present invention relates to the use of one or more purified somatic cell reprogramming factors, alone or together with one or more effectors of cellular metabolism. Methods for reprogramming somatic cells are provided. More particularly, methods for generating induced pluripotent stem cells (iPSCs) from somatic cells are provided. The invention further concerns the reprogrammed cells generated by the methods provided herein.

### BACKGROUND OF THE INVENTION

Embryonic stem (ES) cells have the ability to grow indefinitely while maintaining pluripotency (Evans et al. (1981), Nature 292:154-156). Because of this, human embryonic stem cells may find use in basic and applied research as well as in tissue replacement therapies, for example the treatment of spinal cord injury, as well as other personalized medicine applications.

Although the application of ES cells in the treatment of disease and injury is a promising field of research, there are ethical concerns with the use of ES cells. Additionally, it has been difficult to generate ES cells in the laboratory (see, e.g., U.S. Patent Application Publication No. 2010/0062533). Furthermore, prior to the present invention, methods used to generate iPSCs have all required the use of viruses, genetic integration and/or plasmid vectors, each of which present a variety of serious biological and regulatory obstacles for clinical applications of iPSCs. Because of these issues, researchers have looked into methods for dedifferentiation of somatic cells into induced pluripotent stem cells (iPS(s).

The first reported attempt to reprogram somatic cells utilized retroviruses to express Klf4, Oct4, Sox2 and c-Myc (Takahashi and Yamanaka (2006), Cell, 126:663-76). However, the genomes of the reprogrammed cells contained viral DNA, which could result in deleterious genetic consequences. A number of recent studies have been reported to address this issue by using non-integrating adenovirus, lentiviruses (Sommer et al. (2009), Stem cells, 27:543-9) transient expression vectors (Okita et al. (2008), Science 322:949-53) and targeted integration and excision of vector sequences Kaji, (2009) Nature, 458:771-775; Woltjen et al. (2009) Nature, 458:776-770). However, all these approaches involved viruses, genetic integration, or plasmid vectors, and therefore, presented a variety of biological and regulatory obstacles for clinical applications of iPSCs.

iPSCs are thought to have many of the same capabilities of ES cells. A pluripotent stem cell (PSC) has the potential to differentiate into any of the three germ layers: (1) endoderm (interior stomach lining, gastrointestinal tract, lungs), (2) mesoderm (muscle, bone, blood vessels, urogenital tissue) and (3) ectoderm (epidermal tissues and nervous system). Therefore, methods for generating these cells would greatly benefit the fields of stem cell biology and personalized medicine.

The present invention provides reliable methods for reprogramming somatic cells, for example, to a pluripotent state, without the need for the introduction of viral DNA or other expression vectors or genetic means into the somatic cell(s) to be reprogrammed (i.e., dedifferentiated).

### SUMMARY OF THE INVENTION

Methods are provided herein to for reprogramming (dedifferentiating) somatic cells. More particularly, methods are provided to generate one or more iPSCs from one or more somatic cells. Because DNA or RNA vectors are not used in the methods of the invention, there is no risk for DNA mutation when employing the methods. To the inventors' knowledge, prior to the present invention, no one had published research regarding the reprogramming of mammalian, including human, somatic cells with defined non-genetic (e.g., protein) factors. With relatively small amounts of purified proteins, the methods of the present invention provide the highest efficiency and the fastest reprogramming of somatic cells shown to date. Accordingly, the methods provided herein provide an advantage over the prior art.

In one embodiment, a method for reprogramming a somatic cell is provided, the method comprises growing a somatic cell culture and treating the somatic cell culture with at least one purified somatic cell reprogramming factor. In another embodiment, the method comprises growing a somatic cell culture and treating the somatic cell culture with at least two purified somatic cell reprogramming factors. In another embodiment, the method comprises growing a somatic cell culture and treating the somatic cell culture with at least three purified somatic cell reprogramming factors. In another embodiment, the method comprises growing a somatic cell culture and treating the somatic cell culture with at least four purified somatic cell reprogramming factor. In another embodiment, the method comprises growing a somatic cell culture and treating the somatic cell culture with more than four purified somatic cell reprogramming factors. In a further embodiment, the at least one reprogrammed somatic cell is an iPSC.

In one embodiment, the method comprises growing a somatic cell culture; treating the somatic cell culture with a solution comprising an effective amount of at least one purified somatic cell reprogramming factors; harvesting the treated somatic cell culture to form a cell culture; and growing the cell culture until at least one somatic cell has been reprogrammed. In a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours or about 48 hours apart, in this embodiment. In a further embodiment, the at least one purified somatic cell reprogramming factors is selected from Oct4, Sox2, Klf4, c-Myc, Utf1, AID, M(H)DM2, dominant negative p53, tetramerization domain, p53 inhibitors, and their protein family members. In a further embodiment, the at least one purified somatic cell reprogramming factor is part of a chimeric protein, and is operatively linked to a protein transduction domain, to facilitate cellular entry of the at least one purified somatic cell reprogramming factor. In some embodiments, the protein transduction domain is HIV-TAT or variant thereof. HIV-TAT in one embodiment, is operatively linked to the N-terminus of the at least one purified somatic cell reprogramming factor. In a further embodiment, the at least one reprogrammed somatic cell is an iPSC.

In one embodiment, the method comprises growing a somatic cell culture; treating the somatic cell culture with a solution comprising an effective amount of at least two purified somatic cell reprogramming factors; harvesting the treated somatic cell culture to form a cell culture; and growing the cell culture until at least one somatic cell has been reprogrammed. In a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours or about 48 hours apart, in this embodiment. In a further embodiment, the at least two purified somatic cell reprogramming factors are selected from Oct4, Sox2, Klf4, c-Myc, Utf1, AID, M(H)DM2, dominant negative p53, tetramerization domain, p53 inhibitors, and their protein family members. In a further embodiment, the at least two purified somatic cell reprogramming factors are part of a chimeric protein, and is operatively linked to a protein transduction domain, to facilitate cellular entry of the at least two purified somatic cell reprogramming factors. In some embodiments, the protein transduction domain is HIV-TAT or variant two purified somatic cell reprogramming factors. In a further embodiment, the at least one reprogrammed somatic cell is an iPSC.

In one embodiment, the method comprises growing a somatic cell culture; treating the somatic cell culture with a solution comprising an effective amount of at least three purified somatic cell reprogramming factors; harvesting the treated somatic cell culture to form a cell culture; and growing the cell culture until at least one somatic cell has been reprogrammed. In a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours or about 48 hours apart, in this embodiment. In a further embodiment, the at least two purified somatic cell reprogramming factors are selected from Oct4, Sox2, Klf4, c-Myc, Utf1, AID, M(H)DM2, dominant negative p53, tetramerization domain, p53 inhibitors, and their protein family members. In one embodiment, the at least three purified somatic cell reprogramming factors are Sox2, KLF4 and Oct4, In a further embodiment, the at least three purified somatic cell reprogramming factors are pan of a chimeric protein, and is operatively linked to a protein transduction domain, to facilitate cellular entry of the at least three purified somatic cell reprogramming factors. In some embodiments, the protein transduction domain is HIV-TAT or variant thereof. HIV-TAT, in one embodiment, is operatively linked to the N-terminus of the at least three purified somatic cell reprogramming factors. In a further embodiment, the at least one reprogrammed somatic cell is an iPSC.

In one embodiment, the method comprises growing a somatic cell culture; treating the somatic cell culture with a solution comprising an effective amount of at least four or more purified somatic cell reprogramming factors; harvesting the treated somatic cell culture to form a cell culture; and growing the cell culture until at least one somatic cell has been reprogrammed. In a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours or about 48 hours apart, in this embodiment. In a further embodiment, the at least four purified somatic cell reprogramming factors are selected from Oct4, Sox2, Klf4, c-Myc, Utf1, AID, M(H)DM2, dominant negative p53, tetramerization domain, p53 inhibitors, and their protein family members. In a further embodiment, the at least four purified somatic cell reprogramming factors are part of a chimeric protein, and is operatively linked to a protein transduction domain, to facilitate cellular entry of the at least four purified somatic cell reprogramming factors. In some embodiments, the protein transduction domain is HIV-TAT or variant thereof, HIV-TAT, in one embodiment, is operatively linked to the N-terminus of the at least four purified somatic cell reprogramming factors. In a further embodiment, the at least one reprogrammed somatic cell is an iPSC.

In one embodiment, the method comprises growing a somatic cell culture; treating the somatic cell culture with a solution comprising (1) an effective amount of at least one purified somatic cell reprogramming factor and (2) an effective amount of a reprogramming enhancing factor harvesting the treated somatic cell culture to form a treated somatic cell suspension; plating the treated somatic cell suspension to form a cell culture, and growing the cell culture until at least one somatic cell has been reprogrammed. In a further embodiment, the at least one reprogrammed somatic cell is an iPSC.

In another embodiment, the method comprises growing a somatic cell culture; treating the somatic cell culture with a solution comprising (1) an effective amount of at least two purified somatic cell reprogramming factor and (2) an effective amount of a reprogramming enhancing factor harvesting the treated somatic cell culture to form a treated somatic cell suspension; plating the treated somatic cell suspension to form a cell culture, and growing the cell culture until at least one somatic cell has been reprogrammed, in a further embodiment, the at least one reprogrammed somatic cell is an iPSC.

In a further embodiment, the method comprises growing a somatic cell culture; treating the somatic cell culture with a solution comprising (1) an effective amount of at least three purified somatic cell reprogramming factors and (2) an effective amount of a reprogramming enhancing factor harvesting the treated somatic cell culture to form a treated somatic cell suspension; plating the treated somatic cell suspension to form a cell culture, and growing the cell culture until at least one somatic cell has been reprogrammed. In a further embodiment, the at least one reprogrammed somatic cell is an iPSC.

In a further embodiment, the method comprises growing a somatic cell culture; treating the somatic cell culture with a solution comprising (1) an effective amount of at least four purified somatic cell reprogramming factors and (2) an effective amount of a reprogramming enhancing factor; harvesting the treated somatic cell culture to form a treated somatic cell suspension; plating the treated somatic cell suspension to form a cell culture, and growing the cell culture until at least one somatic cell has been reprogrammed. In a further embodiment, the at least one reprogrammed somatic cell is an iPSC.

In a further embodiment, the method comprises growing a somatic cell culture; treating the somatic cell culture with a solution comprising (1) an effective amount of more than four purified somatic cell reprogramming factors and (2) an effective amount of a reprogramming enhancing factor harvesting the treated somatic cell culture to form a treated somatic cell suspension; plating the treated somatic cell suspension to form a cell culture, and growing the cell culture until at least one somatic cell has been reprogrammed, in a further embodiment, the at least one reprogrammed somatic cell is an iPSC.

In one embodiment, the method comprises growing a somatic cell culture; treating the somatic cell culture with a solution comprising (1) an effective amount of at least one purified somatic cell reprogramming factor and (2) an effective amount of a a somatic cell reprogramming enhancing factor; harvesting the treated somatic cell culture to form a treated somatic cell suspension; plating the treated somatic cell suspension to form a cell culture, and growing the cell culture until at least one somatic cell has been reprogrammed. In a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours or about 48 hours apart, in this embodiment. In a further embodiment, the at least one reprogrammed somatic cell is an iPSC.

In another embodiment, the method comprises growing a somatic cell culture; treating the somatic cell culture with a solution comprising (1) an effective amount of at least one purified somatic cell reprogramming factor and (2) an effective amount of a histone deacetylase inhibitor, harvesting the treated somatic cell culture to form a treated somatic cell suspension; plating the treated somatic cell suspension to form a cell culture, and growing the cell culture until at least one somatic cell has been reprogrammed. In a further embodiment, the at least one reprogrammed somatic cell is an iPSC.

In a further embodiment, the method comprises growing a somatic cell culture; treating the somatic cell culture with a solution comprising (1) an effective amount of at least one purified somatic cell reprogramming factor and (2) an effective amount of a histone deacetylase inhibitor; harvesting the treated somatic cell culture to form a treated somatic cell suspension; plating the treated somatic cell suspension to form a cell culture, and growing the cell culture until at least one somatic cell has been reprogrammed, in a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours, about 48 hours apart or more than 48 hours apart, in this embodiment. In a further embodiment, the at least one reprogrammed somatic cell is an iPSC.

In another embodiment, a method for reprogramming a somatic cell is provided. The method comprises growing a somatic cell culture; treating the somatic cell culture with a solution comprising (1) an effective amount of at least one purified somatic cell reprogramming factor and (2) an effective amount of a histone deacetylase inhibitor selected from valproic acid (VPA), suberoylanitide hydroxamic acid (SAHA) and trichostatin A (TSA); harvesting the treated somatic cell culture to form a treated somatic cell suspension; plating the treated somatic cell suspension to form a cell culture, and growing the cell culture until at least one somatic cell has been reprogrammed. In a further embodiment, the somatic cell culture is a mammalian cell culture. In a further embodiment, the at least one reprogrammed somatic cell is an iPSC. In yet a further embodiment, the histone deacetylase inhibitor is VPA. In yet a further embodiment, the treating step comprises one, two, three, four or more individual treatments. The treatments, in this embodiment, are spaced either about 6 hours, about 12 hours, about 18 hours, about 24 hours, about 48 hours apart or more than 48 hours apart. In a further embodiment, the at least one reprogrammed somatic cell is an iPSC.

In another embodiment, a method for reprogramming a somatic cell is provided. The method comprises growing a somatic cell culture; treating the somatic cell culture with a solution comprising (1) an effective amount of at least two purified somatic cell reprogramming factors and (2) an effective amount of a histone deacetylase inhibitor selected from valproic acid (VPA), suberoylanilide hydroxamic acid (SAHA) and trichostatin A (TSA); harvesting the treated somatic cell culture to form a treated somatic cell suspension; plating the treated somatic cell suspension to form a cell culture, and growing the cell culture until at least one somatic cell has been reprogrammed. In a further embodiment, the somatic cell culture is a mammalian cell culture. In a further embodiment, the at least one reprogrammed somatic cell is an iPSC. In yet a further embodiment, the histone deacetylase inhibitor is VPA. In yet a further embodiment, the treating step comprises one, two, three, four or more individual treatments. The treatments, in this embodiment, are spaced either about 6 hours, about 12 hours, about 18 hours, about 24 hours, about 48 hours apart or more than 48 hours apart. In a further embodiment, the at least one reprogrammed somatic cell is an iPSC.

Another embodiment is directed to a method for reprogramming a somatic cell. The method comprises growing a somatic cell culture; treating the somatic cell culture with a solution comprising (1) an effective amount of at least three purified somatic cell reprogramming factors and (2) an effective amount of a histone deacetylase inhibitor selected from valproic acid (VPA), suberoylanilide hydroxamic acid (SAHA) and trichostatin A (TSA); harvesting the treated somatic cell culture to form a treated somatic cell suspension; plating the treated somatic cell suspension to form a cell culture, and growing the cell culture until at least one somatic cell has been reprogrammed. In a further embodiment, the at least three purified somatic cell reprogramming factors are selected from Oct4, Sox2, Klf4, c-Myc, Utf1, AID, M(H)DIV12, dominant negative p53, tetramerization domain, p53 inhibitors and their protein family members. In even a further embodiment, the method comprises treating the somatic cell culture with at least four of the aforementioned purified somatic cell reprogramming factors. In one embodiment, the at least three purified somatic cell reprogramming factors are Sox2, KLF4 and Oct4. In a further embodiment, the at least one reprogrammed somatic cell is an iPSC.

In yet another embodiment, the present invention is directed to a method for reprogramming one or more somatic cells. The method comprises growing a somatic cell culture; treating the somatic cell culture with a solution comprising (1) an effective amount of at least three purified somatic cell reprogramming factors and (2) an effective amount of a histone deacetylase inhibitor selected from valproic acid (VPA), suberoylanilide hydroxamic acid (SAHA) and trichostatin A (TSA); wherein the treating step comprises one, two, three, four or more individual treatments with each purified somatic cell reprogramming factor spaced about 12 hours, about 24 hours or about 48 hours apart; harvesting the treated somatic cell culture to form a treated somatic cell suspension; plating the treated somatic cell suspension to form a cell culture, and growing the cell culture until at least one somatic cell has been reprogrammed. In a further embodiment, the solution comprises an effective amount of sodium azide. In a further embodiment, the at least one reprogrammed somatic cell is an iPSC.

In one embodiment, the present invention provides a method for generating an iPSC from a somatic cell, which in some embodiments, is a human somatic cell. The method comprises growing a somatic cell culture to at least 25% confluence; treating the somatic cell culture with a solution comprising (1) an effective amount of at least one purified somatic cell reprogramming factor, (2) an effective amount of VPA, (3) sodium azide and (4) vitamin C; harvesting the treated somatic cell culture to form a treated somatic cell suspension; plating the treated somatic cell suspension on a layer or partial layer of feeder cells to form a cell culture, and growing the cell culture until at least one induced pluripotent stem cell is generated. In a further embodiment, the at least one purified somatic cell reprogramming factor is part of a chimeric protein, and is operatively linked to a protein transduction domain, to facilitate cellular entry of the at least one purified somati cell reprogramming factor. In some embodiments, the protein transduction domain is FITV-TA717 or variant thereof. HIV-TAT, in one embodiment, is operatively linked to the N-terminus of the at least one purified somatic cell reprogramming factor.

In a further embodiment, the method comprises growing a somatic cell culture; treating the somatic cell culture with a solution comprising (1) an effective amount of at least one purified somatic cell reprogramming factor and (2) an effective amount of a factor which mimics hypoxia, upregulates glycolysis or inhibits respiration wherein the factor which mimics hypoxia; harvesting the treated somatic cell culture to form a treated somatic cell suspension; plating the treated somatic cell suspension to form a cell culture, and growing the cell culture until at least one somatic cell has been reprogrammed. In a further embodiment, the somatic cell culture is a mammalian cell culture. In yet a further embodiment, the somatic cell culture is a human cell culture. In even a further embodiment, the treating step comprises one, two, three, four or more individual treatments. The treatments are spaced at either about 6 hours, about 12 hours, about 18 hours, about 24 hours, about 48 hours apart or more than 48 hours apart, in this embodiment. In a further embodiment, the at least one reprogrammed somatic cell is an iPSC.

In another embodiment, a method for reprogramming a somatic cell is provided. The method comprises growing a somatic cell culture; treating the somatic cell culture with a solution comprising (1) an effective amount of at least one purified somatic cell reprogramming factor and (2) an effective amount of a factor which mimics hypoxia, upregulates glycolysis or inhibits respiration wherein the factor which mimics hypoxia, upregulates glycolysis or inhibits respiration is sodium azide; harvesting the treated somatic cell culture to form a treated somatic cell suspension; plating the treated somatic cell suspension to form a cell culture, and growing the cell culture until at least one somatic cell has been reprogrammed. In a further embodiment, the somatic cell culture is a mammalian cell culture. In a further embodiment, the at least one reprogrammed somatic cell is an iPSC. In yet a further embodiment, the treating step comprises one, two, three, four or more individual treatments. The treatments, in this embodiment, are spaced either about 6 hours, about 12 hours, about 18 hours, about 24 hours, about 48 hours apart or more than 48 hours apart. In a further embodiment, the at least one reprogrammed somatic cell is an iPSC.

In another embodiment, a method for reprogramming a somatic cell is provided. The method comprises growing a somatic cell culture; treating the somatic cell culture with a solution comprising (1) an effective amount of at least two purified somatic cell reprogramming, factors and (2) an effective amount of a factor that mimics hypoxia, upregulates glycolysis or inhibits respiration; harvesting the treated somatic cell culture to form a treated somatic cell suspension; plating the treated somatic cell suspension to form a cell culture, and growing the cell culture until at least one somatic cell has been reprogrammed. In a further embodiment, the somatic cell culture is a mammalian cell culture. In a further embodiment, the at least one reprogrammed somatic cell is an iPSC. In yet a further embodiment, the a factor that mimics hypoxia, upregulates glycolysis or inhibits respiration wherein the factor which mimics hypoxia is sodium azide. In yet a further embodiment, the treating step comprises one, two, three, four or more individual treatments. The treatments, in this embodiment, are spaced either about 6 hours, about 12 hours, about 18 hours, about 24 hours, about 48 hours apart or more than 48 hours apart. In a further embodiment, the at least one reprogrammed somatic cell is an iPSC.

Another embodiment is directed to a method for reprogramming a somatic cell. The method comprises growing a somatic cell culture; treating the somatic cell culture with a solution comprising (1) an effective amount of at least three purified somatic cell reprogramming factors and (2) an effective amount of a factor that mimics hypoxia, upregulates glycolysis or inhibits respiration; harvesting the treated somatic cell culture to form a treated somatic cell suspension; plating the treated somatic cell suspension to form a cell culture, and growing the cell culture until at least one somatic cell has been reprogrammed. In a further embodiment, the at least three purified somatic cell reprogramming factors are selected from Oct4, Sox2, Klf4, c-Myc, Utf1, AID, M(1-1)DM2, dominant negative p53, tetramerization domain, p53 inhibitors, and their protein family members. In even a further embodiment, the method comprises treating the somatic cell culture with at least four of the aforementioned purified somatic cell reprogramming factors. In one embodiment, the at least three purified somatic cell reprogramming factors are Sox2, KLF4 and Oct4. In a further embodiment a factor that mimics hypoxia, upregulates glycolysis or inhibits respiration wherein the factor which mimics hypoxia is sodium azide. In a further embodiment, the at least one reprogrammed somatic cell is an iPSC.

In yet another embodiment, the present invention is directed to a method for reprogramming one or more somatic cells. The method comprises growing a somatic cell culture; treating the somatic cell culture with a solution comprising (1) an effective amount of at least three purified somatic cell reprogramming factors and (2) an effective amount of a factor that mimics hypoxia, upregulates glycolysis or inhibits respiration; wherein the treating step comprises one, two, three, four or more individual treatments with each purified somatic cell reprogramming factor spaced about 12 hours, about 24 hours or about 48 hours apart; harvesting the treated somatic cell culture to form a treated somatic cell suspension; plating the treated somatic cell suspension to form a cell culture, and growing the cell culture until at least one somatic cell has been reprogrammed. In a further embodiment, the solution comprises an effective amount of sodium azide. In a further embodiment, the at least one reprogrammed somatic cell is an iPSC.

In one embodiment, the present invention provides a method for generating an iPSC from a somatic cell, which in sonic embodiments, is a human somatic cell, The method comprises growing a somatic cell culture to at least 25% confluence; treating the somatic cell culture with a solution comprising (1) an effective amount of at least one purified somatic cell reprogramming factor, (2) an effective amount of VPA, (3) sodium azide and (4) vitamin C; harvesting the treated somatic cell culture to form a treated somatic cell suspension; plating the treated somatic cell suspension on a layer or partial layer of feeder cells to form a cell culture, and growing the cell culture until at least one induced pluripotent stem cell is generated. In a further embodiment, the at least one purified somatic cell reprogramming factor is part of a chimeric protein, and is operatively linked to a protein transduction domain, to facilitate cellular entry of the at least one purified somati cell reprogramming factor. In some embodiments, the protein transduction domain is HIV-TAT or variant thereof. HIV-TAT, in one embodiment, is operatively linked to the N-terminus of the at least one purified somatic cell reprogramming factor.

These and other embodiments are disclosed or are apparent from, and encompassed by, the following Detailed Description.

### DETAILED DESCRIPTION OF THE INVENTION

### Definitions

“Somatic cell,” as used herein, refers to a cell that forms, in part, the body of an organism. Examples of somatic cells include, but are not limited to, fibroblasts, blood cells, epithelial cells, lung cells, glia, neurons, adipose cells, and liver cells.

“Reprogramming a somatic cell,” as used herein, means dedifferentiating a somatic cell into a cell that can either self renew (i.e., a unipotent cell), or a cell that can differentiate into one or more cell types (i.e., a stem cell). For the purposes of this invention, one or more somatic cells can be reprogrammed to one or more pluripotent stem cells, one or more multipotent stem cells (e.g., a hematopoietic cell), one or more oligopotent stem cells, or one or more unipotent cells. A “reprogrammed somatic cell” as used herein mean, a dedifferentiated somatic cell into a cell that can either self renew (i.e., a unipotent cell), or a cell that can differentiate into one or more cell types (i.e., a stem cell). For the purposes of this invention, one or more somatic cells can be reprogrammed to one or more pluripotent stem cells, one or more multipotent stem cells (e.g., a hematopoietic cell), one or more oligopotent stem cells, or one or more unipotent cells.

“Somatic cell reprogramming factor” as used herein means any protein, or peptide fragment thereof which is capable of reprogramming of a somatic cell to a more undifferentiated state.

“Somatic cell reprogramming enhancing factor” as used herein means any molecule, when used in conjunction with a somatic cell reprogramming factor, capable of enhancing the reprogramming of a somatic cell to a dedifferentiated state (e.g., increasing the efficiency, speed or reliability of reprogramming).

A “pluripotent stem cell,” (PSC) is a cell that has the potential to differentiate into a cell present in any of the three germ layers: (1) endoderm, (2) mesoderm or (3) ectoderm.

A “totipotent” or “omnipotent” stem cell, as used herein, can differentiate into an embryonic and extraembryonic cell (e.g., zygote).

The term “nucleic acid molecule” or “polynucleotide” refers to a deoxyribonucleotide or ribonucleotide polymer in either single-stranded or double-stranded form, and, unless specifically indicated otherwise, encompasses polynucleotides containing known analogs of naturally occurring nucleotides that can function in a similar manner as naturally occurring nucleotides. It will be understood that when a nucleic acid molecule is represented by a DNA sequence, this also includes RNA molecules having the corresponding RNA sequence in which “U” (uridine) replaces “T” (thymidine).

The term “recombinant nucleic acid molecule” refers to a laboratory produced nucleic acid molecule. A recombinant nucleic acid molecule can be produced by recombination methods, particularly genetic engineering techniques, or can be produced by a chemical synthesis method. A recombinant nucleic acid molecule can encode a fusion protein, for example, a somatic cell reprogramming factor (or fragment thereof) of the invention linked to a protein transduction domain (PTD). The term “recombinant host cell” refers to a cell that contains a recombinant nucleic acid molecule.

Reference to a polynucleotide “encoding” a polypeptide means that, upon transcription of the polynucleotide and translation of the mRNA produced there from, a polypeptide is produced. The encoding polynucleotide includes the coding strand, whose nucleotide sequence is identical to an mRNA, as well as its complementary strand.

The term “expression control sequence” refers to a nucleotide sequence that regulates the transcription or translation of a polynucleotide or the localization of a polypeptide to which to which it is operatively linked. Expression control sequences are “operatively linked” when the expression control sequence controls or regulates the transcription and, as appropriate, translation of the nucleotide sequence (i.e., a transcription or translation regulatory element, respectively), or localization of an encoded polypeptide to a specific compartment of a cell. Thus, an expression control sequence can be a promoter, enhancer, transcription terminator, a start codon (ATG), a splicing signal for intron excision and maintenance of the correct reading frame, a STOP codon, a ribosome binding site, or a sequence that targets a polypeptide to a particular location, for example, a cell compartmentalization signal (e.g., a protein transduction domain or a cell penetrating peptide), which can target a polypeptide to the cytosol, nucleus, plasma membrane, endoplasmic reticulum, mitochondrial membrane or matrix, chloroplast membrane or lumen, medial trans-Golgi cisternae, or a lysosome or endosome.

The terms “operatively linked” or “operably linked,” as used herein, are synonymous when used herein to describe chimeric proteins, and refer to polypeptide or peptide sequences that are placed in a physical and functional relationship to each other. In a preferred embodiment, the functions of the polypeptide components of the chimeric protein are unchanged compared to the functional activities of the parts in isolation. For example, a somatic cell reprogramming factor of the invention, or variant thereof, can be operatively linked to a protein transduction domain, a peptide tag and/or a fluorescent protein (e.g., GFP). Operatively linked polypeptides, in one embodiment, are produced using recombinant DNA methodologies and then purified, for example, on a nickel chromatography column. In another embodiment, the portions of the chimeric protein are synthesized separately, for example by recombinant DNA methodologies or solid state peptide synthesis, and then linked to each other using peptide bond chemistry.

The terms “amino” and “amine” both refer to an group.

The term “carboxyl” refers to the group —CO₂H and consists of a carbonyl and a hydroxyl group (C(═O)OH).

An “amino acid” is a molecule containing an amino group and carboxyl group, and is typically represented as follows:

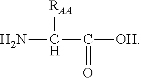

R_{AA} is referred to as the amino acid side chain. Cyclic amino acids do not fall under this formula, as each includes a cyclic group, in addition to the amino and carboxyl moieties. In some instances, the amino or carboxyl group may form part of the cyclic structure (for example, see proline's structure). The somatic cell reprogramming factors of the present invention can comprise both proteinogenic and non-proteinogenic amino acids. The twenty two proteinogenic amino acids (Table 1) are used during protein biosynthesis, and can be incorporated during translation.

TABLE 1
Proteinogenic Amino Acids and Their Abbreviations

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |
| Selenocysteine | SEC | U |
| Pyrrolysine | PYL | O |

A “non-proteinogenic amino acid” is an organic compound which is an amino acid, but is not among those encoded by the standard genetic code, or incorporated into proteins during translation. A non-proteinogenic amino acid may be formed by post-translational modification of a proteinogenic amino acid (for example, the hydroxylation of proline to form hydroxyproline). Other examples of non-proteinogenic amino acids include the D-isosteromers of the proteinogenic amino acids. Further examples of non-proteinogenic amino acids include, but are not limited to the following: citrulline, homocitrulline, hydroxyproline, homoarginine, homoserine, homotyrosine, homoproline, ornithine, 4-amino-phenylalanine, sarcosine, biphenylalanine, homophenylalanine, 4-amino-phenylalanine, 4-nitro-phenylalanine, 4-fluoro-phenylalanine, 2,3/1,5,6-pentafluoro-phenylalanine, norleucine, cyclohexylalanine, α-aminoisobutyric acid, N-methyl-alanine, N-methyl-glycine, N-methyl-glutamic acid, tert-butylglycine, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, cyclohexylglycine, diethylglycine and dipropylglycine.

The terms “polypeptide” and “protein” are synonymous, and refer to a polymer of two or more amino acid residues. The proteins provided herein may include one or more non-proteinogenic amino acids. Preferably, the polypeptide is a polymer of proteinogenic amino acids. These terms also include proteins that are post-translationally modified through reactions that include glycosylation, acetylation and phosphorylation.

The term “recombinant protein,” as used herein, refers to a protein that is produced by expression of a nucleotide sequence encoding the amino acid sequence of the protein from a recombinant DNA molecule.

The terms “isolated” and “purified” as used herein, are synonymous, and refer to a material that is substantially or essentially free from other components. For example, in one embodiment, a recombinant protein is isolated or purified when it is free from other components used in the cloning reaction, or solid state synthesis, isolation or purity is generally determined using analytical chemistry techniques such as polyacrylamide gel electrophoresis, mass spectrometry, or high performance liquid chromatography (HPLC). In one embodiment, a polynucleotide, protein or peptide of the present invention is considered to be isolated when it is the predominant species present in a preparation. A purified protein, peptide or nucleic acid molecule of the invention represents greater than about 80% of the macromolecular species present, greater than about 90% of the macromolecular species present, greater than about 95% of the macromolecular species present, greater than about 96% of the macromolecular species present, greater than about 97% of the macromolecular species present, greater than about 98% of the macromolecular species present, greater than about 99% of the macromolecular species present in a preparation. In a particular embodiment, a purified polynucleotide, protein or peptide is a polynucleotide, protein or peptide purified to essential homogeneity such that it is the only species detected when examined using conventional methods for determining purity of such a molecule.

A “variant” polypeptide, “variant” peptide and “variant” polynucleotide are substantially identical in sequence (e.g., at least 80% sequence identity) to the respective comparison sequence. In a preferred embodiment, the comparison sequence is the native (wild type) polypeptide, peptide or polynucleotide sequence. The variants may contain alterations in the nucleotide and/or amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both. In addition, the nucleotides can be sequenced to ensure that the correct coding regions were cloned and do not contain any unwanted mutations.

Functional fragments and variants of a polypeptide include those fragments and variants that maintain one or more functions of the parent polypeptide. It is recognized that the gene or cDNA encoding a polypeptide can be considerably mutated without materially altering one or more the polypeptide's functions. First, the genetic code is well-known to be degenerate, and thus different codons encode the same amino acids. Second, even where an amino acid substitution is introduced, the mutation can be conservative and have no material impact on the essential function(s) of a protein. See, e.g., Stryer Biochemistry 3^{-rd} Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al (1997) J. Immunol. 159(5): 2502-12). Other modifications that can be made without materially impairing one or more functions of a polypeptide include, for example, in vivo or in vitro chemical and biochemical modifications or the incorporation of unusual amino acids. Such modifications include, but are not limited to, for example, acetylation, carboxylation, phosphorylation, glycosylation, ubiquination, labeling, e.g., with radionucleotides, and various enzymatic modifications, as will be readily appreciated by those well skilled in the art. A variety of methods for labeling polypeptides, and labels useful for such purposes, are well known in the art, and include radioactive isotopes such as P³², ligands which bind to or are bound by labeled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues.

“Effective amount,” as used herein, means an amount of the particular component sufficient to result in the desired response. For example, an effective amount of a purified somatic cell reprogramming protein, in one embodiment, is an amount sufficient to generate an IPSC. However, the response can be any response that a user will recognize as an effective response. Non-limiting examples of responses include (1) generation of an iPSC, (2) generation of a differentiated cell from an iPSC, (3) therapeutic response to a cellular therapy. The “effective amount” may be an amount added at multiple stages over a period of time.

### Reprogramming Factors of the Invention

The present inventors have found, surprisingly, that one or more reprogramming factors can be used, alone, or together with other elements, to dedifferentiate one or more somatic cells, for example to a pluripotent, multipotent or oligopotent state. In a particular embodiment, mammalian iPSCs are generated from somatic cells, without the need to insert viral DNA into the somatic cells. Any factor capable of reprogramming a somatic cell may be used in accordance with the present invention. A non limiting list of reprogramming factors amenable use with the present invention inicude: POU class 5 homeobox 1 (“Pou5fl,” also reffered to herein as “Oct4”); Kruppel-like factor 4 (“Klf4”, for example Klf2, Klf4 or Klf5); sex determining region (Y)-box 2 (“Sox2”); myc proto-oncogene protein (“c-Myc”); dominant negative p53; the murine double minute oncogene (mdm2) and its human counterpart (hdm2); undifferentiated embryonic cell transcription factor 1 (UTF1); SALL4A, SALL4B, Nanog, BMP4, Essrb, AID, Lif, Lin28, M(H)DM2, dominant negative p53, tetramerization domain, p53 inhibitors, and their protein family members.

The mRNA and amino acid sequences of the above factors are known in the art, and are available, for example, in the National Center for Biotechnology Information (NCBI) databases. Table 2 includes a non-limiting representation of the nucleotide and protein accession numbers, corresponding to sequences amenable for use in the methods of the invention. One of ordinary skill in the art, equipped with the sequence accession numbers provided in Table 2 and in the example section (infra), or simply the name of a gene that has previously been sequenced, can synthesize the corresponding cDNA, for example, by solid state methods (i.e., chemical synthesis), or by reverse transcribing the mRNA. The cDNA can then be ligated into an expression vector and cloned, as discussed in more detail below.

TABLE 2
Non-limiting list of sequence accession nos. for human pluripotent
factors of the invention.

| Somatic cell | mRNA sequence | amino acid sequence |
|---|---|---|
| reprogramming factor | accession no. | accession no. |
| Oct4 | NM_002701 | NP_002692 |
| Klf4 | NM_004235 | NP_004226 |
| Sox2 | NM_003106 | NP_003097 |
| c-Myc | NM_002467 | NP_002458 |

In one embodiment, human protein sequences are used in the methods of the invention. However, the invention is not limited to human sequences. The present invention includes the use of any mammalian sequence of the somatic cell reprogramming factors described herein, human or otherwise (e.g., mouse).

In one embodiment, a variant of one or more of the somatic cell reprogramming factors is used in the methods of the invention. Variants can be made, for example, by site directed mutagenesis. The technique is well known in the art (see, e.g., Carter et al., (1985), Nucleic Acids Res. 13:4431-4443 and Kunkel et al. (1987), Proc. Natl. Acad. Sci. USA 82:488). Briefly, in carrying out site directed mutagenesis of DNA, the starting DNA is altered by first hybridizing an oligonucleotide encoding the desired mutation to a single strand of the starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated into the resulting double-stranded DNA. The resulting DNA can then be inserted into a protein expression vector to make the corresponding protein.

Alternatively, protein variants can be made by cassette mutagenesis (see Wells et al. (1985). Gene 34:315-323). In this embodiment, the starting material is an expression vector comprising the starting DNA to be mutated. The codon(s) in the DNA to be mutated are identified. There must be a unique restriction endonuclease site on each side of the identified mutation site(s). If no such restriction sites exist, they may be generated using the above-described site mediated mutagenesis method to introduce them at appropriate locations in the starting DNA. The vector DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using techniques known in the art of molecular biology. This double-stranded oligonucleotide is referred to as the cassette. The cassette is designed to have 5′ and 3′ ends that are compatible with the ends of the linearized plasmid, such that it can be directly ligated to the plasmid. Once ligation is complete, the plasmid contains the mutated DNA sequence.

In one embodiment of the invention, one or more fragments of the purified somatic cell reprogramming factors (or variant(s) thereof) is used in the methods provided herein.

### Preparation of the Reprogramming Factors of the Invention

Upon selection of individual factor(s), variant(s), fragment(s), homolog(s), ortholog(s) or family member(s), the somatic cell reprogramming factors are synthesized by methods well known to those skilled in the art of molecular biology and/or solid state chemistry.

If the DNA sequence of the reprogramming factor is known, it can be synthesized commercially. For example, sequences can be submitted to BlueHeron® Biotechnology (Bothell, Wash.) or DNA2.0 (Menlo Park, Calif.), for commercial synthesis of the DNA. The corresponding proteins can then be made by methods well known in the art of molecular biology.

In one embodiment, somatic cell reprogramming factor cDNA is inserted into an expression vector for cloning and protein expression. The vector, in one embodiment, includes an expression control sequence such as a transcription regulatory element. In one embodiment, the transcription regulatory element is a promoter or a polyadenylation signal sequence. In another embodiment, the expression control sequence is a translation regulator element such as a ribosome binding site.

The vector generally contains elements required for replication in a prokaryotic or eukaryotic host system or both, as desired. In one embodiment, the somatic cell reprogramming factors described herein are expressed in mammalian host cells. The vectors of the invention, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (for example, Promega, Madison Wis.; Stratagene, La Jolla GIBCO/BRL, Gaithersburg Md.) or can be constructed by one skilled in the art (see, e.g., Meth. Enzymol., Vol. 185, Goeddel, ed. (Academic Press, Inc., 1990); Jolly (1994), Canc. Gene Ther. 1:51-64; Flotte (1993), Bioenerg. Biomemb, 25:37-42; Kirshenbaum et al. (1992), J. Clin. Invest. 92:381-387).

The construction of expression vectors and the expression of a polynucleotide in transfected cells involves the use of molecular cloning techniques also well known in the art (see Sambrook et al., In “Molecular Cloning: A Laboratory Manual” (Cold Spring Harbor Laboratory Press 1989); “Current Protocols in Molecular Biology” (eds., Ausubel et al.; Greene Publishing Associates, Inc., and John Wiley & Sons, Inc. 1990 and supplements)). Expression vectors, in one embodiment, contain expression control sequences linked to a polynucleotide sequence of interest, for example, a polynucleotide encoding for a somatic cell reprogramming factor of interest (or variant or fragment thereof). The expression vector (for example, pCR4Blunt-TOPO (Invitrogen, Carlsbad, Calif.)) can be adapted for function in prokaryotes or eukaryotes by inclusion of appropriate promoters, replication sequences, markers, and the like. An expression vector can be transfected into a recombinant host cell for expression of the somatic cell reprogramming factor protein, and host cells can be selected, for example, for high levels of expression in order to obtain a large amount of isolated protein. A host cell can be maintained in cell culture, or can be a cell in vivo in an organism. A somatic cell reprogramming factor (or variant or fragment thereof) can be produced by expression from a polynucleotide encoding the protein in a host cell such as E. coli, yeast cells or insect cells. Alternatively, in one embodiment, the somatic cell reprogramming factor can be expressed in a mammalian host cell.

The protein expression vectors of the invention can include additional sequences to allow for the expressed somatic cell reprogramming factor (or variant thereof) to be linked to one or more polypeptides or peptides of interest. This linkage occurs by inserting the DNA corresponding to the polypeptide or peptide of interest at the 5″ or 3′ end of the somatic cell reprogramming factor DNA, in a protein expression vector. For example, in one embodiment, the somatic cell reprogramming factor can be linked to a protein transduction domain (PTD) peptide, discussed further below. In another embodiment, the somatic cell reprogramming factor is operatively linked only to a peptide tag, used for protein purification. It will be understood by those of ordinary skill in the art that the peptide purification tag is used solely for protein purification, and once the purification step is complete, the peptide purification tag is cleaved from the remainder of the protein.

In one embodiment, there are two peptides of interest linked to the expressed the somatic cell reprogramming factor (or variant thereof). The first peptide of interest is a protein transduction domain peptide and the second peptide of interest is a peptide tag (i.e., a purification tag), which can be used to facilitate isolation of the somatic cell reprogramming, factor, including any other polypeptides linked thereto (e.g., a protein transduction domain peptide). In this embodiment, the protein transduction domain is present at the C-terminal or N-terminal end of the somatic cell reprogramming factor. Alternatively, the protein transduction domain is present at an internal portion of the somatic cell reprogramming factor. Internal fusion is carried out, in one embodiment, if it does not impact the catalytic and/or regulatory activities of the active somatic cell reprogramming factor. As the regulatory and catalytic domains of a protein typically constitute a very small part of the respective full length protein, there is an ample sequence space for the internal fusion.

There may also be a spacer sequence between the protein transduction domain and the somatic cell reprogramming factor. The peptide tag, in this embodiment, is either linked to the end of the somatic cell reprogramming factor not linked to the protein transduction domain, or is linked to the free end of the protein transduction domain.

The peptide purification tag can be a polyhistidine tag containing, for example, six histidine residues, and as stated above, can be incorporated at the N-terminus of the somatic cell reprogramming factor (or variant thereof), the C-terminus, or can be present as an internal sequence. The somatic cell reprogramming actor can then be isolated from the remainder of a sample, for example, by nickel-chelate chromatography. Alternatively, as described above, the peptide purification tag can be incorporated at the free end of the protein transduction domain. Additional peptide purification tags, including streptavidin, glutathione S-transferase (GST), maltose binding protein (MBP), thioredoxin, a FLAG epitope, or any other ligand, including any peptide epitope (or antibody, or antigen binding fragment thereof, that specifically binds the epitope are well known in the art and similarly can be used (see, e.g., Hopp et al. (1988), Biotechnology 6:1204; U.S. Pat. No. 5,011,912).

The somatic cell reprogramming factors described herein, in some embodiments, may be purified without the use of peptide purification tags. For example, electrofocusing, on exchange chromatography and gel filtration chromatography may all be used to purify the reprogramming factors of the invention. These techniques are well known in the art of biochemistry and molecular biology (see, e.g., Goldman and Babtist (1979), J. Chromatogr. 179:330-332; Irvine (2001), Curr Protoc Cell Biol. May; Chapter 5:Unit 5.5; Suck et al., (2006), J. Biotechnol. 121:361-367; Calogero and Aulicino (2004), Methods Mol Med. 94:225-238).

In some embodiments, it may be desirable to fluorescently label the somatic cell reprogramming factor. Labeling can be performed (1) after the protein has been purified, with a chromophore, or (2) by joining a fluorescent protein to the somatic cell reprogramming factor to form a chimeric protein. For example, the green fluorescent protein (UT) sequence can be inserted directly upstream or downstream of the somatic cell reprogramming factor's DNA in an expression vector. The fluorescent chimera, in one embodiment, further includes a peptide tag for protein purification. In a further embodiment, the fluorescent chimera contains a peptide tag and a PTD peptide. Non-limiting examples of chimeric proteins of the present invention are provided below, in Table 3.

TABLE 3
Non-limiting list of chimeric proteins amenable
for use with the present invention

NH₂-(peptide purification tag)-(PTD peptide)-(somatic cell reprogramming
factor)-CO₂H
NH₂-(peptide purification tag)-(somatic cell reprogramming factor)-
(PTD peptide)-CO₂H
NH₂-(PTD peptide)-(somatic cell reprogramming factor)-(peptide
purification tag)-CO₂H
NH₂-(peptide purification tag)-(somatic cell reprogramming factor)-CO₂H
NH₂-(somatic cell reprogramming factor)-(peptide purification tag)-CO₂H
NH₂-(fluorescent protein)-(somatic cell reprogramming factor)-(peptide
purification tag)-CO₂H
NH₂-(peptide purification tag)-(somatic cell reprogramming factor)-
(fluorescent protein)-CO₂H
NH₂-(peptide purification tag)-(PTD peptide)-(somatic cell reprogramming
factor)-(fluorescent protein)-CO₂H
NH₂-(peptide purification tag)-(somatic cell reprogramming factor)-(PTD
peptide)-(fluorescent protein)-CO₂H
NH₂-(fluorescent protein)-(PTD peptide)-(somatic cell reprogramming
factor)-(peptide purification tag)-CO₂H
NH₂-(fluorescent protein)-(somatic cell reprogramming factor)-(peptide
purification tag)-CO₂H
NH₂-(PTD peptide)-(somatic cell reprogramming factor)-CO₂H
NH₂-(somatic cell reprogramming factor)-(PTD peptide)-CO₂H

As an alternative to recombinantly expressing the one or more somatic cell reprogramming factors of the invention, it may be desirable to synthesize the factor chemically, e.g., by liquid phase or solid-phase synthesis. Accordingly, both techniques can be employed to prepare the one or more somatic cell reprogramming factors of the invention. Solid-phase synthesis may be particularly useful when introducing non-proteinogenic amino acids into the one or more somatic cell reprogramming factors. The solid phase method, in one embodiment, is employed when it is difficult to express the protein of interest in a host cell. Solid phase synthesis was described originally by Merrifield (1963), JACS 85:2149. Additionally, chemical protein synthesis is available by commercial vendors, for example by GenScript (Piscataway, N.J.).

In an alternative embodiment, the somatic cell reprogramming factors of the invention are made by in vitro translation methods, also well known in the art. Kits to carry out this technique are available, e.g., from Pierce (a division of Thermo Fisher Scientific Inc., Rockford, Ill.) and Ambion (a division of Applied Biosystems, Austin Tex.). For example, in one embodiment, the somatic cell reprogramming factors of the invention are expressed in a cell-free expression system, e.g., rabbit reticulocyte lysate, wheat germ extract or an E. coli cell-free system. In a further embodiment, the somatic cell reprogramming factors of the invention are translated in a linked transcription:translation system.

### Protein Delivery Systems

The somatic cell reprogramming factors (or variants thereof) described herein are delivered into one or more somatic cells to generate one or more iPSCs. However, the wild type somatic cell reprogramming factors (or variants thereof) require an external delivery system to allow for cellular entry. Accordingly, the somatic cell reprogramming factors described herein can be operatively linked, chemically linked, or recombinantly expressed with, a protein transduction domain (PTD) peptide. In another embodiment, a PTD peptide is not used, and the one or more purified somatic cell reprogramming factors are introduced into one or more cells by electroporation or by incorporation into, liposomes or nanoparticles. These cellular delivery systems are discussed in more detail, below.

### PTD Peptides

PTD peptides are commonly referred to cell penetrating peptides (CPPs). These peptides known in the art are amenable for use with the present invention. For example, in one embodiment, a homopolymer of arginine or lysine, or a heteropolymer of arginine and lysine is operatively linked to a somatic cell reprogramming factor. In this embodiment, the length of the peptide is typically 5 amino acids, 6 amino acids, 7 amino acids, 8 amino acids, 9 amino acids, 10 amino acids, 11 amino acids, 12 amino acids, 13 amino acids, 14 amino acids, 15 amino acids or more, CPPs are described, for example by El-Sayed et al. (2009), AAPS J 11:13-22 and Ziegler et al. (2005), Biochemistry 44:138-148. Other PTD examples are given below.

In another embodiment, a nuclear translocation peptide is operatively linked to the at least one somatic cell reprogramming factor of the invention. In this embodiment, the nuclear translocation peptide can be linked to the somatic cell reprogramming factor in the manners described above for the protein purification tags, i.e., to the N-terminus or C-terminus of the somatic cell reprogramming factor, or at an internal portion of the somatic cell reprogramming factor. In a further embodiment, the chimeric protein includes a cell penetrating peptide, in order to translocate the lipid bilayer membrane. An example of a nuclear translocation peptide amenable for use with the present invention is the SV40 Large T nuclear localization sequence.

### HIV Transactivator Protein (TAT)

In one embodiment, the at least one somatic cell reprogramming factor is operatively linked to the HIV transactivator protein (TAT) peptide, a variant thereof, or a fragment thereof. For example, in one embodiment, the at least one somatic cell reprogramming factor is linked to amino acids 47-57 of the full length TAT protein. In another embodiment, the at least one somatic cell reprogramming factor is linked to a polyTAT sequence—i.e., a peptide comprising at least two repeats of the 47-47 amino acid sequence. Alternatively, the polyTAT sequence can include one or more variants of the TAT peptide.

The TAT peptide sequence (47-57) is given as SEQ ID NO: 1, below. This peptide is available commercially, for example, by Anaspec, Inc. (Fremont, Calif., catalog no. 60023-5).

(SEQ ID NO: 1)
NH₂-Tyr-Gly-Arg-Lys-Lys-Arg-Arg-Gln-Arg-Arg-Arg-
CO₂H.

In another embodiment, the PTD is a TAT variant, and includes at least one additional Arg residue.

The somatic cell reprogramming factors described herein, in one embodiment, are recombinantly expressed, purified, and then chemically linked to one TAT peptide or other PTD peptide or two or more copies of these PTD peptides. In this embodiment, peptide bond formation occurs either (1) between the N-terminus of the pluripotent protein and the C-terminus of the PTD peptide, or (2) between the C-terminus of the pluripotent protein and the N-terminus of the PTD peptide. Alternatively, two peptide bonds are formed, and the PTD peptide is present as an internal sequence of the somatic cell reprogramming factor.

In a preferred embodiment, the DNA corresponding to the somatic cell reprogramming factor is inserted into an expression vector containing the one or more PTD sequence(s) (for example, the pTAT-HA plasmid vector). A linking DNA sequence may be inserted between the somatic cell reprogramming factor DNA and the PTD peptide. In one embodiment, the somatic cell reprogramming factor DNA is inserted at the 5′ end of the one or more PTD DNA sequence(s). In another embodiment, the somatic cell reprogramming factor DNA is inserted at the 3′ end of the one or more PTD DNA sequence(s). Therefore, the one or more PTD sequence(s) may be joined to the C-terminus or N-terminus of the somatic cell reprogramming factor, or may be joined as an internal sequence.

In a specific embodiment, the PTD sequence is TAT, and is joined to the N-terminal end of at least one somatic cell reprogramming factor protein.

### Penetratin™ 1 Peptide

Another PTD peptide amenable for use with the present invention is the Penetratin™ 1 peptide, available for example, from Krackeler Scientific Inc., Albany, N.Y. (see also Perez et al. (1994), Mol. Endocrinol. 8:1278-1287). The peptide is 16 amino acids long and corresponds to the third helix of the homeodomain of antennapedeia protein.

In one embodiment, one or more Penetratinmt I peptide is activated and coupled directly to a somatic cell reprogramming factor of the invention. In another embodiment, the DNA sequence corresponding to the one or more Penetratin™ 1 peptide is inserted in a protein expression vector, either upstream or downstream of the somatic cell reprogramming factor DNA. In a further embodiment, there is a linking sequence between the one or more Penetratin™ 1 DNA and the somatic cell reprogramming factor DNA.

In one embodiment, the Penetratin™ 1 peptide sequence is as follows:

(SEQ ID NO: 2)
NH₂-Arg-Gln-Ile-Lys-Ile-Trp-Phe-Gln-Asn-Arg-Arg-Met-Lys-Trp-Lys-Lys-CO₂H

In one embodiment, the protein expression vector includes the one or more Penetratin™ 1 DNA sequence 5′ or 3′ to the somatic cell reprogramming factor's DNA. In a further embodiment, there is a DNA linking sequence between the Penetratin™ 1 DNA and the somatic cell reprogramming factor's DNA. In these embodiments, the protein expression vector can include a peptide tag, used for purification of the somatic cell reprogramming factor-PTD chimera. The peptide purification tag, for example polyHis, can be used in downstream affinity purification methods, which are well known to those skilled in the art. The peptide tag can be operatively linked to either the Penetratin™ 1 moiety, or to the somatic cell reprogramming factor moiety. In a further embodiment, the chimeric protein also includes a fluorescent moiety (e.g., GFP).

### VP22 Peptide

VP22 is a herpes simplex virus type it (HSV-1) structural protein, and has been shown to traffic between cells in vivo, as well as when part of a GFP⁻fusion protein (Elliott and O'Hare (1997), Cell 88:223-233; Elliott and O'Hare (1999), Gene Therapy 6:149-151). Accordingly, in one embodiment, the one or more purified somatic cell reprogramming factors forms part of a chimeric protein with one or more VP22 protein(s), or a peptide fragment(s) thereof. In a further embodiment, the chimeric protein is purified with a peptide purification tag, for example, a polyHis peptide tag. In a yet a further embodiment, the chimeric protein includes a fluorescent moiety, for example GFP.

Plasmids containing VP22 DNA are available for example, from Invitrogen. One of ordinary skill in the art can readily insert a somatic cell reprogramming factor into such a vector, to recombinantly express the fusion protein (i.e., VP22-somatic cell reprogramming factor chimera). Alternatively, each portion of the chimeric protein can be made separately, and then operatively linked by peptide bond chemistry.

### Histones, Peptide Fragments, and Variants Thereof

The histones (H1, H2A, H2B, H3, H4) have been shown to enter cells through an energy and receptor independent manner (see, e.g., Hariton-Gazal et al. (2003), J Cell Sci. 116:4577-4586; Wagstaff et al. (2007), Molecular Therapy 15:721-731). For example, a peptide derived from H2A has been shown to mediate the transfer of a macromolecule into COS-7 cells (see Balicki et al. (2002), Proc. Natl. Acad. Sci. USA 97:11500-11504). Accordingly, in one embodiment, one of the histone proteins (or peptide fragment thereof) can be linked to the somatic cell reprogramming factors of the present invention and delivered into one or more somatic cells.

In one embodiment, the linkage between a histone or peptide fragment and a somatic cell reprogramming factor can occur as described above (for example, in a protein expression vector using the histone DNA, or chemically linking the historic protein to the reprogramming factor after the latter has been recombinantly expressed, cloned and purified).

The histone or peptide fragment can be operatively linked to the N-terminus or C-terminus of the somatic cell reprogramming factor. Alternatively, the historic or peptide fragment can be internally linked to the somatic cell reprogramming factor (i.e., as a sequence between the N- and C-termini). In one embodiment, there is a linking sequence between the two aforementioned moieties. In a further embodiment, a fluorescent moiety is included in the aforementioned chimeric protein.

In another embodiment, the chimeric histone-somatic cell reprogramming factor moiety is operatively linked to a peptide tag, for example a poly-His sequence, for downstream protein purification on a nickel affinity chromatography column. As described above for the other PTD chimeras, the peptide tag can either be operatively linked to the PTD moiety, or to the somatic cell reprogramming factor moiety. In some embodiments, there is a linking sequence between the peptide tag and the PTD-somatic cell reprogramming factor chimeric protein.

### Non-PTD Delivery Systems

### Electroporation

As an alternative to the use of a PTD moiety, the one or more somatic cell reprogramming factors may be introduced into one or more cells by electroporation (see, e.g., Marreo et al. (1995), J. Biol. Chem., 270:15734-15738; Nolkrantz et al. (2002), Anal, Chem. 74:4300-4305). Briefly, in this embodiment, somatic cells are placed in a pulsed electrical field, and high-voltage electric pulses result in the formation of pores within the lipid bilayer cell membranes. Proteins can then enter the cells through the pores.

In one embodiment, the voltage applied to the cell suspension is 1 pulse at 10 volts, 1 pulse at 20 volts, 1 pulse at 30 volts, 1 pulse at 40 volts, 1 pulse at 50 volts, 1 pulse at 60 volts, 1 pulse at 70 volts, 1 pulse at 80 volts, 1 pulse at 90 volts, 1 pulse at 100 volts, 1 pulse at 110 volts, 1 pulse at 120 volts, 1 pulse at 130 volts, 1 pulse at 140 volts or 1 pulse at 150 volts. In another embodiment, the voltage applied to the cell suspension is 2 pulses at 10 volts each, 2 pulses at 20 volts each, 2 pulses at 30 volts each, 2 pulses at 40 volts each, 2 pulses at 50 volts each, 2 pulses at 60 volts each, 2 pulses at 70 volts each, 2 pulses at 80 volts each, 2 pulses at 90 volts each, 2 pulses at 100 volts each, 2 pulses at 110 volts each, 2 pulses at 120 volts each, 2 pulses at 130 volts each, 2 pulses at 140 volts each or 2 pulses at 150 volts each,

In one electroporation embodiment, mammalian cells, for example fibroblast cells, are suspended in a buffered solution of the one or more purified proteins of interest. The suspension is placed in a pulsed electrical field, and high-voltage electric pulses result in the formation of pores within the lipid bilayer cell membrane.

In another embodiment, cells are electroporated in tissue culture dishes using a Petri dish electrode. In one embodiment the electrode is 100 mm in diameter with 2-mm gap electrodes. However, other Petri dish electrodes are amenable for use with the present invention. In the culture dish electroporation embodiments, the one or more somatic cell reprogramming factors of interest are included in the electroporation medium, for example Ca²⁺ and Mg²⁺-free Hank's balanced salt solution.

After electroporation, the cell culture dish or cells in the cell suspension can be incubated at 37° C. (5% CO₂) for 5 minutes, 10 minutes, 15 minutes 20 minutes, 25 minutes or 30 minutes.

In one cell tissue culture electroporation embodiment, after incubating the cells as described above, the plates are washed once with serum-free DMEM (Dulbecco's Modified Eagle's Medium) and further incubated in serum-free DMEM for 30 minutes at 37° C.

In a cell suspension electroporation embodiment, after incubating the cell suspension as described above, the cells are washed with DMEM by first pelleting the cells by centrifugation, followed by the addition of serum-free DMEM to the pellet. After washing the cell pellet, the cells are suspended in serum-free DMEM for 30 minutes at 37° C.

### Liposomes

In one embodiment, the purified somatic cell reprogramming factors of the invention are delivered into one or more somatic cells by liposome carriers. Liposomes, in one embodiment, are made of lipids. In a further embodiment, the lipids are phospholipids. In another embodiment, the liposomes employed in the present invention are cationic.

Liposomes are formed, in one embodiment, by adding a solution of lipids (or phospholipids) to the solution of protein or proteins to be delivered intracellularly. The solution is then sonicated or mixed.

Somatic cell reprogramming factors delivered into cells by liposomes, in one embodiment, have a nuclear localization sequence operatively linked thereto. Alternatively or additionally, the protein has a PTD peptide operatively linked thereto.

Procedures for encapsulating proteins in liposomes are well known in the art. As an example, the ordinary skilled artisan is directed to Zelphati et al. (2001), J. Biol. Chem. 276:35103-35110.

### Nanoparticles

The purified somatic cell reprogramming factors of the present invention can also be delivered into mammalian cells with inorganic nanoparticles. For example, in one embodiment, particles made from calcium phosphate, gold, silver, platinum, palladium, iron-gold alloy, iron-platinum alloy, transition metal chalcogenides passivated by zinc sulfide, carbon materials, silicon oxide, iron oxide or layered double hydroxide (LDH) are employed as cellular delivery agents. In the nanoparticle embodiments, described herein, the somatic cell reprogramming factor may or may not have a PTI) peptide operatively linked thereto.

In one embodiment, the individual nanoparticles used in the present invention are between nm in diameter and 1000 nm (1 μm) in diameter.

Spacers, in one embodiment, are employed to link the somatic cell reprogramming factors of the present invention to nanoparticles. The spacer molecule, in a specific embodiment, is selected from a molecule with a thiol group, homo-bifunctional polyethylene oxides, hetero-bifunctional polyethylene oxides, a peptide and functionalized oligonucleotides.

Preparation of nanoparticles for cellular delivery of proteins is given, for example, U.S. Patent Application Publication No. 2009/0098574, the teachings of which are incorporated by reference, herein, in its entirety.

### Somatic Cell Reprogramming Enhancing Factors

### Histone Deacetylase Inhibitors

The present inventors have found that a histone deacetylase (HDAC) inhibitor, together with the one or more purified somatic cell reprogramming factors (described above), greatly improves the reprogramming efficiency of somatic cells, as compared to the sole introduction of one or more purified somatic cell reprogramming factors. In the HDAC embodiments, the HDAC inhibitor is present in a solution containing the one or more purified somatic cell reprogramming factors, and cells are exposed to the solution. The solution is typically a solution of cell culture medium.

Examples of HDAC inhibitors amenable for use with the present invention include valproic acid (VPA), suberoylanilide hydroxamic acid (SAHA) and trichostatin A (TSA). Each of these HDAC inhibitors is commercially available, for example from EMD Biosciences, San Diego, Calif.; Biomol International, Plymouth Meeting, Pa. or Sigma-Aidrich St. Louis, Mo. (See, for example, Fluangfu et al. (2008), Nat. Biotechnol. 26:795-797).

In one embodiment, an HDAC inhibitor is used together with the one or more purified somatic cell reprogramming factors, and the HDAC inhibitor is VPA. In a further embodiment, the concentration of VPA is about 0.5 mM, about 1 mM, about 2 mM, about 4 mM, about 6 mM, about 8 mM, about 10 mM, about 12 mM, about 14 mM, about 16 mM, about 18 mM or about 20 mM. In another embodiment, the concentration of VPA is either about 1 mM or about 2 mM.

In another embodiment, SAHA is used with the one or more purified somatic cell reprogramming factors. In a further embodiment, the concentration of SAHA is about 1 μM, about 2 μM, about 3 μM, about 4 μM, about 5 μM, about 6 μM, about 7 μM, about 8 μM, about 9 μM, about 10 μM, or about 20 μM.

In another embodiment, TSA is used with the one or more purified somatic cell reprogramming factors. In a further embodiment, the concentration of ISA is about 1 nM, about 5 nM, about 10 nM, about 15 nM, about 20 nM, about 25 nM, about 30 nM, about 40 nM or about 50 nM.

In another embodiment, two HDAC inhibitors are used together with the one or more purified somatic cell reprogramming factors. In a further embodiment, the two HDAC inhibitors are (1) VPA and SAHA, (2) VPA and TSA or (3) TSA and SAHA.

In even another embodiment, the three aforementioned HDAC inhibitors are used together with the one or more purified somatic cell reprogramming factors.

Alternatively or additionally, the methods of the invention can be carried out with at least one histone acetyl transferase. Examples of histone acetyl transferases amenable for use with the present invention are CREBBP, CDY1, CDY2, CDYL1, CLOCK, ELP3, EP300, HAT1, TF3C4, NCO and MYST (1-4).

### Hypoxia Conditions

The methods of the invention, in one embodiment, are carried out under conditions that upregulate glycolysis, inhibit respiration, hypoxic conditions, or conditions mimicking hypoxia. A consequence of the chemical hypoxia is an upregulation of glycolysis (Naughton (2003), Medical Hypotheses 60:332-334). Accordingly, in one embodiment, the methods provided herein are carried out under conditions which upregulate glycolysis. Alternatively or additionally, the methods provided herein are carried out in conditions which inhibit cellular respiration.

Sodium azide (NaN₃) has been reported to induce hypoxia (see, e.g., Gramniatopoulos et al. (2004), Brain Research Bulletin 62:297-303). It is thought that sodium azide blocks the oxygen-requiring steps in energy metabolism by inhibition of cytochrome oxidase and, accordingly, induces a “chemical hypoxia” (Rose et al. (1998), J. Neurosci. 18:3554-3567).

In one embodiment of the invention, sodium azide is added to a solution of the one or more purified somatic cell reprogramming factors. In a further embodiment, the solution contains one or more HDAC inhibitors, as described above. In a further embodiment, the solution contains vitamin C. In one embodiment, sodium azide is present at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, or about 2 mM. In one embodiment, sodium azide is introduced into the one or more somatic cells with a solution of 0.002% sodium azide, for a final concentration of 0.3 mM sodium azide. Alternatively, sodium azide is introduced into the one or more somatic cells with a solution of about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, or about 0.009%.

In one embodiment, the one or more purified somatic cell reprogramming factors is used in conjunction with sodium azide. In another embodiment, the one or more purified somatic cell reprogramming factors is used in conjunction with VPA and sodium azide to reprogram a somatic cell. In a further embodiment, the reprogrammed somatic cell is one or more iPSCs.

In an alternative embodiment, hypoxia can be mimicked by limiting the oxygen exposure of the cells, for example by culturing the cells in a low-oxygen or hypoxia chamber. This can occur before, during or after treatment of the somatic cell culture with the one or more purified somatic cell reprogramming factors.

In another embodiment, cellular respiration is inhibited chemically by an agent other than sodium azide. For example, malonate can be employed in the methods of the present invention. Other cellular respiratory inhibitors are known in the art of molecular biology and biochemistry, and are amenable for use with the present invention.

In one embodiment, the methods of the present invention are carried out under normal oxygen conditions where glycolysis has been upregulated. For example, glycolysis can be upregulated by the introduction of the myc protein (or variant thereof) or sodium azide. Alternatively or additionally, glycolysis can be increased by the introduction of dominant negative p53 (see Molchadsky et al. (2008), PLoS One 3:e3707), or transducible Ras and/or Akt proteins or tactors which upregulate Ras and/or Akt.

### Vitamin C

Vitamin C has been reported to enhance the reprogramming of somatic cells to pluripotent stem cells (Esteban et al. (2010), Cell Stem Cell 6:71-79). However, vitamin C, to the inventors' knowledge, has not been used in a solution comprising one or more purified somatic cell reprogramming factor proteins, in order to reprogram one or more somatic cells to one or more iPSCs. Nor has vitamin C been reported to have been used in a solution comprising one or more purified somatic cell reprogramming factor proteins and a histone deacetylase inhibitor to reprogram one or more somatic cells.

In one embodiment, the invention is directed to a method of reprogramming generating one or more somatic cells comprising treating the one or more somatic cells, with one or more purified somatic cell reprogramming factors and vitamin C. In a further embodiment, the method includes contacting the one or more somatic cells for a sufficient period of time with one or more purified somatic cell reprogramming factors, and vitamin C. In a further embodiment, the method includes contacting the one or more somatic cells for a sufficient period of time with one or more purified somatic cell reprogramming factors, one or more somatic cell reprogramming enhancing factors and vitamin C. In a further embodiment, the method includes contacting the one or more somatic cells for a sufficient period of time with one or more purified somatic cell reprogramming factors, a histone deacetylase and vitamin C. In a further embodiment, the method includes contacting the one or more somatic cells for a sufficient period of time with one or more purified somatic cell reprogramming factors, VPA and vitamin C. In a further embodiment, the method includes contacting the one or more somatic cells for a sufficient period of time with one or more purified somatic cell reprogramming factors, VPA, sodium azide and vitamin C.

In one embodiment, the concentration of vitamin C used in the treating step is about 10 μg/mL, about 15 μg/mL, about 20 μg/mL, about 25 about 30 μg/mL, about 35 μg/mL, about 40 μg/mL, about 45 μg/mL, about 50 μg/mL, about 55 about 60 μg/mL, about 65 μg/mL, about 70 or about 75 μg/mL. In one embodiment, the final concentration of vitamin C in a treating step is about 5 μM, about 10 μM, about 15 μM or about 20 μM. Multiple treatments of vitamin C may be employed with the vitamin C concentrations described herein.

In one embodiment, the treating step comprises multiple treatments. If the treating step comprises multiple treatments, for example one or more treatments occurring about 12 hours, 1 day, about 2 days, about 3 days, about 4 days, about 5 day, about 6 days, about 7 days or more after the initial treatment, the concentration of vitamin C used in the second treatment, in one embodiment, is the same as the concentration used in the second treatment. In another embodiment, the concentration of vitamin C is increased in the second treatment, as compared to the first treatment. In yet another embodiment, the concentration of vitamin C is decreased in the second treatment, as compared to the first treatment. In one embodiment, the methods of the invention can include three, four or five treatments of vitamin C. This aspect of the invention is discussed in more detail, below.

### Additional Reprogramming Enhancers

Other factors have been reported in the literature to increase the reprogramming efficiency of somatic cells. However, these factors, to the inventors' knowledge, have not been disclosed or suggested for use with the methods or the purified reprogramming factors described herein.

Accordingly, in one embodiment, the following reprogramming enhancing factors factors can be used with the methods of the present invention, to enhance the reprogramming of one or more somatic cells.

SV40 large T antigen, (Mali, et al. (2008), Stem cells 26:1998-2005 and Park, et al. (2008), Nature, 451; 141-146); catalytic subunit of human telomerase (hTERT) (Mali, et al. (2008) Stem cells 26:1998-2005 and Park, et al. (2008), Nature, 451; 141-146); inhibitors of DNA methyltransferase e.g. RG108 (Shi, et al. (2008), Cell Stem Cell, 3: 568-574) and AZA (Mikkelson et al. (2008), Nature, 454:49-55); MEK, inhibitors, e.g., PD0325901. (Silva et al., (2008), PLos Biol. 6, e253 10.371/journal.pbio.0060253); GSK3 inhibitors e.g., CHR99021 . . . . Silva et al, (2008), PLos Biol. 6, e253, 10.371/journalpbio.0060253); TGFβ inhibitor e.g., A-83-01 (Li et al. (2009), Cell Stem Cell 4:46-19); Effectors of Writ signaling (Tcf3, Cole et al. (2008), Genes Dev. 22:746-0.755); Utf1. (Zhao, et al. (2008), Cell Stem Cell, 3, 475-479); G9a histone methyltransferase inhibitors e.g., BIX-10294 (Shi, et al. (2008), Cell Stem Cell, 3: 568-574) and L-type calcium channel agonists e.g., BayK8644 (Shi, et al. (2008), Cell Stem Cell, 3: 568-574); DNA demethylase e.g. AID (Bhutani, et al. (2010) Nature, 463:1042-1048). Alternatively or additionally, inhibitors of DNA methylation, e.g., 5-aza-deoxycytidine (Huangfu, et al. (2008), Nat. Biotechnol., 26:795-7) can be employed.

### METHODS OF THE INVENTION

The present inventors have surprisingly found that a somatic cell can be reprogrammed without genetic manipulation. Because DNA or RNA vectors are not being used in the methods of the invention, there is no risk for genetic mutation when treating the somatic cells.

The methods provided herein utilize a somatic cell culture and purified proteins, alone or with one or more reprogramming enhancing factors, to reprogram at least one somatic cell, for example to produce an iPSC. Although the invention is mainly described using a fibroblast as the somatic cell, the invention is not limited thereto. Any somatic cell is amenable for use with the present invention.

In the methods disclosed herein, the somatic cells are plated in an appropriate medium and allowed to adhere to the plate and grow, at least overnight for at least 8 hours) Cell growth, in one embodiment, takes place in a 37° C./5% CO₂ incubator. However, other CO₂ and O₂ concentrations can be used.

Cells can be grown until at least 5% confluent, at least 10% confluent, at least 15% confluent, at least 20% confluent, at least 25% confluent, at least 30% confluent, at least 35% confluent, at least 40% confluent, at least 45% confluent, at least 50% confluent, at least 60% confluent, at least 65% confluent, at least 70% confluent, at least 75% confluent, at least 80% confluent, at least 85% confluent, at least 90% confluent, at least 95% confluent or 99% confluent.

Once a cell culture reaches the desired confluence, it is treated with at least one purified somatic cell reprogramming factor protein. As described above, the purified somatic cell reprogramming factor may or may not be operatively linked to a PTD domain. This will depend on the protein delivery system chosen by the user. In one embodiment, the cell culture medium is replaced with fresh medium prior to treatment. In another embodiment, the medium is not replaced.

In one embodiment, the purified somatic cell reprogramming factor is diluted in a buffer, for example, buffer Z, prior to cell culture treatment.

The initial cell culture treatment with at least one somatic cell reprogramming factor may be accompanied by an additional treatment with one or more compounds/factors that have a role in cellular metabolism. For example, valproic acid, sodium azide and vitamin C may be added to the cell culture upon treatment with the purified somatic cell reprogramming factor(s). In another embodiment, valproic acid and sodium azide are added, white vitamin C is not. In yet another embodiment, vitamin C or valproic acid is the only compound added with the initial protein treatment. These compounds, in one embodiment, are added directly to culture medium, e.g., by pipetting.

Once the initial treatment is complete, cell cultures are incubated in an incubator, for example a 37° C./5% CO₂ incubator. In one embodiment, the incubation period is about 8 hours, about 9, hours, about 10 hours, about 11 hours, about 12 hours, about 14 hours, about 16 hours, about 17 hours, about 18 hours, about 19 hours, about 20 hours, about 21 hours, about 22 hours, about 23 hours, about 24 hours, about 30 hours, about 32 hours, about 34 hours, about 36 hours, about 38 hours, about 40 hours, about 42 hours, about 44 hours, about 46 hours, about 48 hours, about 60 hours, about 72 hours, about 96 hours, about 120 hours, about 144 hours (about 6 days), about 168 hours (about 7 days), about 192 hours (about 8 days) or about 216 hours (about 9 days).

In one embodiment, after the incubation period, one or more additional treatments are carried out. The additional treatments may or may not include one or more purified somatic cell reprogramming factors. For example, a second treatment may be limited to valproic acid and vitamin C.

Upon completion of the initial incubation period, the treated somatic cells can be treated at additional times, as described further below. In one embodiment, the cells, after treatment(s) are completed, are allowed to grow until at least one somatic cell has been reprogrammed, for example until at least one iPSC has been generated. In another embodiment, after the one or more treatments are completed, the treated somatic cells are harvested as a cell suspension, and added to a cell culture containing feeder cells, or a culture comprising extracellular matrix proteins.

### Non Fee Cell Embodiments

In one embodiment, once the somatic cell treatment (or multiple treatments) is complete, and the cells have been incubated for the desired amount of time, the treated somatic cells can be harvested to form a somatic cell suspension. In one embodiment, the treated somatic cell suspension is replated, and grown in a cell culture containing the necessary constituents to allow for cell maintenance without the presence of feeder cells. In one embodiment the cell cuture comprises LIE and STAT3 (see, e.g., Williams et al. (1988), Nature 336:684 and Raz et al. (1999), Proc Natl. Acad. Sci. USA 96:2846)).

In another embodiment, the treated somatic cell suspension is replated, and grown in a cell culture comprising an inhibitor of glycogen synthase kinase (GSK-3) inhibitor. In a further embodiment, the GSK-3 inhibitor is 6-Bromoindirubin-3′-oxime (BIO), available, for example, from Tocris Bioscience (Ellisville, Mo.). This inhibitor has been shown to maintain embryonic stem cells in the undifferentiated state (Sato and Brivanlou (2006), Methods in Molecular Biology 331:115-128, ISBN 978-1-58829-497-5 (Print) 978-1-59745-046-1 (Online)).

In yet another embodiment, the treated somatic cell suspension is replated, and grown in a cell culture comprising medium supplemented with 15% serum replacement, a combination of growth factors including transforming growth factor beta1 (TGFbeta1), leukemia inhibitory factor, basic fibroblast growth factor, and fibronectin matrix (Shariki et al. (2004), Biol Reprod. 70:837-845).

In one embodiment, the treated somatic cell suspension is replated, and cultured on Matrigel™ (available, for example, from BD Biosciences, Franklin Lakes, N.J.), or laminin coated plates. Matrigel™ comprises mostly a mixture of laminin, collagen IV and heparan sulfate proteoglycan. In the Matrigel™ and laminin embodiments, the medium used for cell culture is conditioned by mouse embryonic fibroblasts (see Xu et al. (2001), Nat. Biotechnol. 19:971-974).

Other feeder free systems are described in the art, for example by Amit (Amit (2007), Methods in Molecular Biology 407:11-20, 978-1-58829-744-0 (Print) 978-1-59745-536-7 (Online)), incorporated herein by reference in its entirety. Embodiments of the present invention include growing a treated somatic cell culture the feeder-free culture systems described by Amit.

### Feeder Cell Embodiments

Upon completion of the initial incubation period, the treated somatic cells can be treated at additional times, as described further below, followed by adding the treated cells to a feeder cell culture or a culture comprising extracellular matrix proteins. Alternatively, the treated somatic cells can be added to a feeder cell culture (or a culture comprising extracellular matrix proteins) for additional growth, after initial treatment and incubation.

In one embodiment, feeder cells, such as human embryonic fibroblasts treated with MITC (HEF-MITC) are plated on gelatin coated cell culture plates and allowed to grow for at least 24 hours in a 37° C./5% CO₂ incubator. In one embodiment, feeder cells are plated at a density of about 0.9×10⁵ cells per well in standard 6-well plates, about 1.0×10⁵ cells per well, about 1.1×10⁵ cells per well, about 1.2×10⁵ cells per well, about 1.3×10⁵ cells per well, about 1.4×10⁵ cells per well, about 1.5×10⁵ cells per well, about 1.6×10⁵ cells per well, about 1.7×10⁵ cells per well, about 1.8×10⁵ cells per well, about 1.9×10⁵ cells per well, about 2.0×10⁵ cells per well, about 2.1×10⁵ cells per well, about 2.2×10⁵ cells per well about 2.3×10⁵ cells per well, about 2.4×10⁵ cells per well, about 2.5×10⁵ cells per well, about 2.6×10⁵ cells per well, about 2.7×10⁵ cells per well, about 2.8×10⁵ cells per well, about 2.9×10⁵ cells per well, about 3.0×10⁵ cells per well or more.

The treated somatic cells (either after initial treatment and incubation, or further treatments and incubations) are dissociated from their respective culture wells, for example with 1×PBS with Ca²⁺ and Mg²⁺ containing collagenase IV, or DMEM/F12 containing collagenase IV. For example, in one embodiment, the concentration of collagenase IV used is about 0.5 mg/mL, about it mg/mL, about 1.5 mg/mL or about 2 mg/mL. In one embodiment, the dissociation takes place at 37° C., and takes approximately 5 minutes. However, this time period will vary depending on the density of the cell cultures and the concentration of collagenase IV.

In another embodiment, the treated somatic cells are dissociated with 0.05% trypsin-EDTA instead of collagenase IV.

Once the treated cells are dissociated, medium, for example, HEScGRO Basal Medium (Millipore, Billerica, Mass.) is added to each well containing a PBS cell suspension. The ordinary skilled artisan will readily know which medium to select based on the specific cell type employed. The cell suspensions are then collected and transferred to separate sterile centrifuge tubes, or alternatively, consolidated and transferred to one sterile centrifuge tube. Consolidation may be desirable when there are a limited number of cells in each well/dish. The cells are then pelleted in a refrigerated (4° C.) centrifuge. In one embodiment, centrifugation for five minutes at 800 rpm is sufficient to pellet the cells. This time may increase for denser suspensions.

The cell pellet is then resuspended in an appropriate volume of medium. In one embodiment, the medium used in this resuspension step is the same medium that was added to the dissociated cells. The cells are then transferred to the already plated feeder cells. In one embodiment, the ratio of treated somatic cells (dissociated cells) to feeder cells is for example, 1:5, 1:4, 1:3, 1:2, 1:1, 2:1, 3:1, 4:1, 5:1 or more.

As described above, the treated somatic cells can be cultured in the presence of feeder cells or in a feeder-free culture. Regardless of the option chosen, after culturing the treated somatic cells, in one embodiment, the cell culture is then incubated in a 37° C./5% CO₂ incubator. In one embodiment, the medium can be supplemented with one or more of the following agents—valproic acid, sodium azide, vitamin C. The cultures are checked for stem cell like colonies regularly, for example daily. In one embodiment, the medium of each culture is changed daily. In one embodiment, it takes about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or more for stem cells to appear. In this embodiment, day 1 is the day of initial treatment with the one or more somatic cell reprogramming factors factor(s).

### Multiple Treatment Steps

The present invention is directed in part, to a method for generating a reprogrammed somatic cell, for example an induced pluripotent stem cell (iPSC). The method comprises treating at least one somatic cell with an effective amount of at least one purified somatic cell reprogramming factor with or without an effective amount of a reprogramming enhancing factor. In one embodiment, the somatic cell is present in a cell culture vessel, and prior to the treating step, the growth medium from the vessel is aspirated from the vessel. Then, medium supplemented with the protein(s) and histone deacetylase inhibitor can be added to the cells. In a further embodiment, the medium is further supplemented with one or more of vitamin C, sodium azide.

In another embodiment, fresh cell culture medium can be added to the cells after an aspiration step, followed by the addition of the purified somatic cell reprogramming factors and optionally one or reprogramming enhancing factor to the medium, by pipetting. Alternatively, in one embodiment, the medium is not replaced prior to a treatment step. In another embodiment, cells are washed prior to at least one of the treatments.

In one embodiment, multiple treatments are included in the treating step, and are carried out to reprogram at least one somatic cell, for example to generate at least one iPSC. For example, in one embodiment, the invention comprises 1, 2, 3, 4, 5, 6, 7, 8, 9, 10 or more treatment steps, spaced at 24 hour intervals. Alternatively, the invention comprises 1, 2, 3, 4, 5, 6, 7, 8, 9, 10 or more treatment steps, spaced at 12 hour intervals. In even another embodiment, the invention comprises 1, 2, 3, 4, 5, 6, 7, 8, 9, 10 or more contacting steps spaced at 6 hour intervals, 12 hour intervals, 18 hour intervals, 24 hour intervals, 30 hour intervals, 36 hour intervals, 42 hour intervals 48 hour intervals or more. The intervals can be varied and need not be the same between each treatment.

In the multiple treatment embodiments, the intervals between each treatment need not consist of the same time period. For example, in one embodiment, the invention comprises 3 treatment steps, and the second treatment is about 24 hours after the first treatment and the third treatment is about 48 hours after the second treatment. The cell culture medium may or may not be changed prior to each treatment.

In a particular embodiment where multiple contacting steps are employed, the specific components, and concentrations of components used in the first treating step (e.g., the at least one purified somatic cell reprogramming factor with or without at least one reprogramming enhancing factor) are the components and concentrations used in the second treatment.

In another embodiment, the components in the second treatment utilizes an additional component not included in the first contacting step, in one embodiment, the additional component is a compound which effects cellular metabolism, or is an additional purified somatic cell reprogramming factor. For example, if the first treatment employs a solution comprising one histone deacetylase inhibitor, the second contacting step, in this embodiment, employs two histone deacetylase inhibitors, or one histone deacetylase inhibitor and vitamin C. Alternatively, a second, third or fourth treatment comprises valproic acid, sodium azide and vitamin C. This concept is further described in the example section of the present specification, and in Table 4, below.

TABLE 4
Non-limiting cell treatment embodiments of the invention

| | treatment 1 | treatment 2 | treatment 3 | treatment 4 | treatment 5 |
|---|---|---|---|---|---|
| embodiment 1 | VPA, Oct4, | VPA, Oct4, | VPA, Oct4, | n/a | n/a |
| | Klf4, Sox2 | Klf4, Sox2 | Klf4, Sox2 | | |
| embodiment 2 | VPA, Sodium | VPA, Sodium | VPA, Sodium | n/a | n/a |
| | Azide, Oct4, | Azide, Oct4, | Azide, Oct4, | | |
| | Klf4, Sox2 | Klf4, Sox2 | Klf4, Sox2 | | |
| embodiment 3 | VPA, Sodium | VPA, Sodium | VPA, Sodium | n/a | n/a |
| | Azide, Vit. C, | Azide, Vit. C, | Azide, Vit. C, | | |
| | Oct4, Klf4, | Oct4, Klf4, | Oct4, Klf4, | | |
| | Sox2 | Sox2 | Sox2 | | |
| embodiment 4 | VPA, Oct4, | VPA, Oct4, | VPA, Oct4, | n/a | n/a |
| | Klf4, Sox2, c- | Klf4, Sox2, c- | Klf4, Sox2, c- | | |
| | Myc | Myc | Myc | | |
| embodiment 5 | VPA, Sodium | VPA, Sodium | VPA, Sodium | n/a | n/a |
| | Azide, Oct4, | Azide, Oct4, | Azide, Oct4, | | |
| | Klf4, Sox2, c- | Klf4, Sox2, c- | Klf4, Sox2, c- | | |
| | Myc | Myc | Myc | | |
| embodiment 6 | VPA, Sodium | VPA, Sodium | VPA, Sodium | n/a | n/a |
| | Azide, Vit. C, | Azide, Vit. C, | Azide, Vit. C, | | |
| | Oct4, Klf4, | Oct4, Klf4, | Oct4, Klf4, | | |
| | Sox2, c-Myc | Sox2, c-Myc | Sox2, c-Myc | | |
| embodiment 7 | VPA, Sodium | VPA, Sodium | VPA, Sodium | n/a | n/a |
| | Azide, Vit. C, | Azide, Vit. C, | Azide, Vit. C, | | |
| | Oct4, Klf4, | Oct4, Klf4, | Oct4, Klf4, | | |
| | Sox2, c-Myc, | Sox2, c-Myc, | Sox2, c-Myc, | | |
| | hdm2 | hdm2 | hdm2 | | |
| embodiment 8 | nanog, Oct4, | nanog, Oct4, | Oct4, c-myc, | n/a | n/a |
| | c-myc, Vit. C, | c-myc, Vit. C, | Vit. C, VPA | | |
| | VPA | VPA | | | |
| embodiment 7 | SAHA, Vit. C, | SAHA, Vit. C, | SAHA, Vit. C, | SAHA, Vit. C, | n/a |
| | Oct4, Klf4, | Oct4, Klf4, | Oct4, Klf4, | Oct4, Klf4, | |
| | Sox2 | Sox2 | Sox2 | Sox2 | |
| embodiment 8 | SAHA, Vit. C, | SAHA, Vit. C, | SAHA, Vit. C, | n/a | n/a |
| | Oct4, Klf4, | Oct4, Klf4, | Oct4, Klf4, | | |
| | Sox2 | Sox2 | Sox2 | | |
| embodiment 9 | Oct4, Klf4, | Oct4, Klf4, | Oct4, Klf4, | Oct4, Klf4, | |
| | Sox2 | Sox2 | Sox2 | Sox2 | |

### Material Transfers

The present invention encompasses embodiments where the method steps are carried out by one or more parties in one or more facilities, Accordingly, in one embodiment, the somatic cell culture is obtained by one party from another party, or transferred from a separate facility, before the culture is treated with the one or more purified somatic cell reprogramming factors. In another embodiment, the somatic cell culture is treated by one party and then transferred to another party or another facility. In yet another embodiment, both of the transfers described above take place.

In one embodiment, once the treated somatic cell culture is harvested, it is transferred to another party or another facility. The latter then grows the treated somatic cell culture in order to reprogram the somatic cell(s).

### Validation Assays

In order to determine whether the generation of iPSCs is successful, the cells are examined for the expression of markers of pluripotency. For example, alkaline phosphatase (AP), stage-specific embryonic antigen-3 (SSEA-3), stage-specific embryonic antigen-3 (SSEA-4) Oct-4, the homeobox protein Nanog, TRA-1-60, Rex1, Gdf3, hTERT, ALP, and ESG1 can each be probed for to determine whether the cells, subjected to the methods described above, are pluripotent.

In one embodiment, the cells subjected to the methods of the invention are immunostained with antibodies specific for the one or more markers given above, to determine whether the methods of the present invention generated iPSCs. Immunofluorescence methods are well known to those of ordinary skill in the art. Additionally, antibodies are commercially available for the above identified markers.

Alternatively or additionally, to determine whether the cells of the invention have pluripotent characteristics, the cells are subjected to fluorescent activated cell sorting (FACS) to determine if any of the above markers are expressed.

Alternatively or additionally, FACS or fluorescence microscopy (or both) can be employed to detect specific proteins expressed in ectoderm, mesoderm and endoderm cells. For example, cell cultures can be probed for nestin (ectoderm), desmine (mesoderm), and hepatocyte necrosis factor (EINF 3β, endoderm). Antibodies for these factors are available commercially, for example from Santa Cruz Biotechnology, Santa Cruz, Calif. or Chemicon, now a part of Millipore (Billerica, Mass.).

In another embodiment, to determine whether iPSCs or other reprogrammed somatic cells have been generated, the cells are lysed, and mRNA isolated, followed by RT-PCR. The PCR is specific for one or more of the somatic cell reprogramming factors given above. In one RT-PCR embodiment, single cell RT-PCR is performed.

In another embodiment, bisulfite genomic sequencing analysis of the Oct4 and/or nanog promoters is employed to detect level of demethylation. Reprogrammed somatic cells have decreased methylation of their stem cell factor promoters compared to MEFs (Zhou et al. (2009), Cell Stem Cell 4:381-384). Accordingly, a decrease in methylation is correlated to the generation of a reprogrammed somatic cell.

### Uses of the Cells of the Invention

The reprogrammed somatic cells of the current invention (for example, the iPSCs) may be further differentiated into endoderm, mesoderm and/or ectoderm tissue. Methods of differentiating pluripotent stem cells are known in the art. For example the reprogrammed somatic cells of the current invention (for example, the iPSCs) derived according to the method of the current invention can be used to generate differentiated neurons according to the methods of Chambers, et al. (2009), Nat. Biotechnol. 27:275-280, hematopoietic or endothelial cells according to the methods of Choi et al. (2009), Stem Cells, 27:559-567, pancreatic insulin producing cells according to the methods of Zhang, et al, (2009), Cell Res. 19:429-438, cardiomyocytes according to the method of Zhang et a. (2009), Circ. Res. 104:e30-41, hepatocyte like cells according to the methods of Song et al. (2009), Cell Res. 19:1233-1242 and retinal cells according to the methods of Meyer, et al. (2009), Proc. Natl. Acad. Aci. USA 106:16698-16703. Without being bound by theory, it is believed that the reprogrammed somatic cells derived in accordance with the methods of the current invention may be differentiated in to any cells of the endoderm, mesoderm and ectoderm layer by any of the methods previously described for pluripotent cells or totipotent embryonic stem cells.

In another aspect of the current invention, the derived differentiated cells may be used for any cellular application for which differentiated cells may be used, including but not limited to cellular assays, for example drug screening assays, disease modeling, or cell replacement therapy. Using the reprogrammed cells, i.e. the iPSC, derived differentiated cells of the current invention provides an advantage over the currently available cellular therapies in that there is no viral integration. DNA or RNA and autologous cells may be used thereby preventing tissue rejection.

### EXAMPLES

The present invention is further illustrated by reference to the following Examples. However, it should be noted that these Examples, like the embodiments described above, are illustrative and are not to be construed as restricting the enabled scope of the invention in any way.

### Materials and Methods

Mitomycin-C was purchased from Sigma (presently catalog no. M4287), reconstituted in water to 1 mg/mL and used immediately. CelLytic™ B Plus Kit was purchased from Sigma Aldrich (presently catalog no. CB0500-1KT). Alkaline phosphatase staining kit was purchased from Millipore and used according to the manufacturer's instructions (presently catalog no, SCR004). Valproic acid sodium salt (presently catalog no. P4543), sodium azide (presently catalog no. S8032) and vitamin C (presently catalog no. A4034) were purchased from Sigma.

### Stem Cell Medium Composition (ES-Cm Medium)

200 mL DMEM/F12 (Invitrogen, presently catalog no. 11320-033)

50 ml FBS ES-Qualified (Fisher Scientific, presently catalog no. SH3007003E) (or 50 mL Knockout)

2.5 mL NEAA (non-essential amino acids) (Invitrogen, presently catalog no. 11140-050)

2.5 mL Glutamine (2 mM) (Invitrogen, presently catalog no. 25030081)

2.5 mL P/S (Invitrogen, presently catalog no, 15070-063)

2 μl BME (TC grade) (VWR, presently catalog no. 95017-124)

500 μL bFGF (4 μg/mL) presently catalog no. GF003)

The medium was filtered with a 0.22 μm sterifilter (Nalgene, presently catalog no. 565-0020).

### Mouse Feeder Cells.

Mouse Feeder (MEF-MITC) cells were obtained from the American Type Culture Collection (“ATCC,” presently catalog no. SCRC-1008.2). Before plating the cells, 0.1% gelatin solution (Millipore EmbryoMax® ES Cell Qualified 0.1% Gelatin Solution, catalog no. ES-006B) was added to individual wells in a 6 well cell culture plate. The plate was then incubated for 45 minutes in a 37° C. CO₂ incubator. Gelatin solution was removed prior to plating cells. MEF-MITC cells were plated at 1.6×10³ cell per well in 15% FBS (VWR, presently catalog no. 95025-546)/DMEM (Invitrogen, presently catalog no. 11995-065) on gelatin coated 6 well plates.

### Human Feeder Cells

A vessel of HIT-1 cells (ATCC presently catalog no. SCRC-1041) were stored in liquid nitrogen until ready for use. The cells were quickly thawed by placing the lower half of the vessel in a 37° C. water bath for 30 seconds to 1 minute. The vessel's outer surfaces were then washed with ethanol. The cells in the vessel were resuspended by gently pipetting the cells with a 2 mL pipette. The cells were then transferred to a 15 mL tube. The cell suspension was then centrifuged for 5 minutes at 800 RPM, in order to pellet the cells. The cell pellet was then resuspended in an appropriate volume of culture medium (DMEM/10% FBS, VAT, catalog no. 21030-CV) supplemented with 1× penicillin-streptomycin (PS)). The suspension was then plated in a cell culture flask. Cells were treated with mitomycin C (final concentration of 10 μg/mL) at passages 3 and 4. The cell culture flasks were then incubated for 3 hours in a 3° C./5% CO₂ incubator.

After the 3 hour incubation, each flask was washed with 10 mL PBS, two times. Trypsin-EDTA (VWR presently catalog no, 4500-662) was added to the flasks to dissociate cells (1 minute in a 37° C./5% CO₂ incubator). An equal amount of culture medium was added to inactivate the trypsin; and cells were gently pipetted up and down to break up any clumps. The cell suspension was transferred to a 50 mL tube and centrifuged at 220×g for 5 minutes at 4° C. The pellet was resuspended in culture medium and the cells were brought to a final concentration of 5×10⁶ cells/mL. An equal volume of 2× freezing medium (20% DMSO, ATcc catalog no. 4-X, 80% FBS, VWR catalog no. 95025-546) was added to the cell suspension to bring the concentration of cells to 2.5×10° cells/mL. The cells were then frozen in a Mr. Frosty container (Nalgenee) the next day, and transferred to liquid nitrogen.

### Mouse Oct4-GFP Embryonic Fibroblast Cells

Mouse embryonic Oct4-GFP⁺ fibroblasts were prepared according to published protocol (see Nature Protocols (2007), 2(12):3081).

A pregnant Oct4-GFP⁺ transgenic female mouse (B6; 129S4-Pou5fl^{tm2Jae}/J, Jackson) (E12.5-13.5) was sacrificed by CO₂ asphyxiation and cervical dislocation. The uterus was isolated and briefly washed with 1×PBS. Ten embryos were separated from the placenta and surrounding membranes with forceps. The head and visceral tissue gonads were removed from the isolated embryos. The embryos were washed with 1×PBS and hashed out with a pair of scissors. Hashed embryonic tissue was transferred into two 50 mL tubes containing 3 mL trypsin-EDTA solution per embryo (5 embryos per tube). Embryo tissues in trypsin-EDTA solutions were incubated at 37° C. for 20 minutes. Additional trypsin-EDTA solution (3 mL/embryo) was added to each 50 mL tube, and the tubes were incubated for another 20 minutes at 37° C.

An equal amount of DMEM and 10% fetal bovine serum (FBS)-penicillin-streptomycin (PS) (FBS-PS) (6 mL/embryo) was added to each tube, and tissues were dissociated by pipetting (at this point, the two tubes were divided into 4 tubes). The tissue suspension was kept at room temperature for 5 minutes to remove debris. The suspension was then decanted into new sterile 50 mL tubes. Decanted medium was centrifuged at 200×g for 5 minutes, and the respective supernatants were discarded. The pellets were resuspended in fresh medium. Cell number was counted and adjusted the concentration to 1×10⁶ cells/mL. The cell suspensions were transferred to 10-cm dishes (1×10⁷ cells/dish). The dishes were incubated at 37° C. with 5% CO₂ for 24 hrs. On the following day, all plates were trypsinized and passaged to 1:4 dilution (Passage 2). After the cells became fully confluent (approximately 2 days), the cells were trypsinized and a frozen stock was prepared in 10% DMSO-15% FBS DMEM. For the generation of iPSCs, the fibroblast cells were prepared from the frozen stock and were immediately used.

### Human Dermal Fibroblast HDF-Neonatal (HDF-n) and HDF-Adult (HDFa) Cells

Human dermal fibroblast HDF-neonatal (HDF-n) and HDF-adult (HDFa) cells were purchased from Cell Applications (catalog nos, 106-05N and 106-05a). HDF cells at passage 4 were used for induction of iPSCs. HDF cells were thawed in cryopreserved vessel of HDF that had been stored in liquid nitrogen. Cells were quickly thawed by placing the lower half of the vessel in 37° C. water bath for 30 seconds to 1 minute. The vessel's outer surface was washed with alcohol. The cells in the vessel were resuspended by gently pipetting the cells up and down with a 2 mL pipette. The cell suspension was transferred to a 15 mL tube, centrifuged for 5 minutes at 800 RPM at 4° C. The cell pellet was subsequently resuspended in an appropriate volume of human fibroblast culture media and plated into wells of 6-well plates. Potential human iPSCs were grown on MEF-MITC or HFE-MITC with ES-cm medium changed daily. HDF cells were maintained in Human Fibroblast Culture Media (Cell Applications, presently catalog no. 116-500).

Oligonucleotides Used for Cloning into pTAT-Ha Vectors

| Mouse OCT4 (POU51) | |
|---|---|
| Primers | |
| (SEQ ID NO: 3) | |
| Xho-Oct4-F: GATCC TCGAG ATGGC TGGAC ACCTG GCTTC AG | |
| (SEQ ID NO: 4) | |
| Eco-Oct4-R: GATCG AATTC TCAGT TTGAA TGCAT GGGAG AGC | |
| (SEQ ID NO: 5) | |
| Mouse OCT4 sequence | |
| 1 | atggctggac acctggcttc agacttcgcc ttctcacccc caccaggtgg gggtgatggg |
| 61 | tcagcagggc tggagccggg ctgggtggat cctcgaacct ggctaagctt ccaagggcct |
| 121 | ccaggtgggc ctggaatcgg accaggctca gaggtattgg ggatctcccc atgtccgccc |
| 181 | gcatacgagt tctgcggagg gatggcatac tgtggacctc aggttggact gggcctagtc |
| 241 | ccccaagttg gcgtggagac tttgcagcct gagggccagg caggagcacg agtggaaagc |
| 301 | aactcagagg gaacctcctc tgagccctgt gccgaccgcc ccaatgccgt gaagttggag |
| 361 | aaggtggaac caactcccga ggagtcccag gacatgaaag ccctgcagaa ggagctagaa |
| 421 | cagtttgcca agctgctgaa gcagaagagg atcaccttgg ggtacaccca ggccgacgtg |
| 481 | gggctcaccc tgggcgttct ctttggaaag gtgttcagcc agaccaccat ctgtcgcttc |
| 541 | gaggccttgc agctcagcct taagaacatg tgtaagctgc ggcccctgct ggagaagtgg |
| 601 | gtggaggaag ccgacaacaa tgagaacctt caggagatat gcaaatcgga gaccctggtg |
| 661 | caggcccgga agagaaagcg aactagcatt gagaaccgtg tgaggtggag tctggagacc |
| 721 | atgtttctga agtgcccgaa gccctcccta cagcagatca ctcacatcgc caatcagctt |
| 781 | gggctagaga aggatgtggt tcgagtatgg ttctgtaacc ggcgccagaa gggcaaaaga |
| 841 | tcaagtattg agtattccca acgagaagag tatgaggcta cagggacacc tttcccaggg |
| 901 | ggggctgtat cctttcctct gcccccaggt ccccactttg gcaccccagg ctatggaagc |
| 961 | ccccacttca ccacactcta ctcagtccct tttcctgagg gcgaggcctt tccctctgtt |
| 1021 | cccgtcactg ctctgggctc tcccatgcat tcaactga |
| Mouse SOX2 | |
| Primers | |
| (SEQ ID NO: 6) | |
| Xho-Sox2-F: GATCC TCGAG ATGTA TAACA TGATG GAGAC G | |
| (SEQ ID NO: 7) | |
| Eco-Sox2-R: GATCG AATTC TCCA TGTGC GACAG GGGCA GTG | |
| (SEQ ID NO: 8) | |
| Mouse SOX2 sequence | |
| 1 | atgtataaca tgatggagac ggagctgaag ccgccggtcc cgcagcaagc ttcggggggc |
| 61 | ggcggcggag gaggcaacgc cacggcggcg gcgaccggcg gcaaccagaa gaacagcccg |
| 121 | gaccgcgtca agaggcccat gaacgccttc atggtatggt cccgggggca gcggcgtaag |
| 181 | atggcccagg agaaccccaa gatgcacaac tcggagatca gcaagcgcct gggcgcggag |
| 241 | tggaaacttt tgtccgagac cgagaagcgg ccgttcatcg acgaggccaa gcggctgcgc |
| 301 | gctctgcaca tgaaggagca cccggattat aaataccggc cgcggcggaa aaccaagacg |
| 361 | ctcatgaaga aggataagta cacgcttccc ggaggcttgc tggcccccgg cgggaacagc |
| 421 | atggcgagcg gggttggggt gggcgccggc ctgggtgcgg gcgtgaacca gcgcatggac |
| 481 | agctacgcgc acatgaacgg ctggagcaac ggcagctaca gcatgatgca ggagcagctg |
| 541 | ggctacccgc agcacccggg cctcaacgct cacggcgcgg cacagatgca accgatgcac |
| 601 | cgctacgacg tcagcgccct gcagtacaac tccatgacca gctcgcagac ctacatgaac |
| 661 | ggctcgccca cctacagcat gtcctactcg cagcagggca cccccggtat ggcgctgggc |
| 721 | tccatgggct ctgtggtcaa gtccgaggcc agctccagcc cccccgtggt tacctcttcc |
| 781 | tcccactcca gggcgccctg ccaggccggg gacctccggg acatgatcag catgtacctc |
| 841 | cccggcgccg aggtgccgga gcccgctgcg cccagtagac tgcacatggc ccagcactac |
| 901 | cagagcggcc cggtgcccgg cacggccatt aacggcacac tgcccctgtc gcacatgtga |
| Mouse KLF4 | |
| Primers | |
| (SEQ ID NO: 9) | |
| Xho-KLF4-F: GATCC TCGAG GCTGT CAGCG ACGCT CTGCT C | |
| (SEQ ID NO: 10) | |
| Eco-Klf4-R: GATCG AATTC TTAAA AGTGC CTCTT CATGT GTAAG | |
| (SEQ ID NO: 11) | |
| Mouse KLF4 sequence | |
| 1 | atgaggcagc cacctggcga gtctgacatg gctgtcagcg acgctctgct cccgtccttc |
| 61 | tccacgttcg cgtccggccc ggcgggaagg gagaagacac tgcgtccagc aggtgccccg |
| 121 | actaaccgtt ggcgtgagga actctctcac atgaagcgac ttcccccact tcccggccgc |
| 181 | ccctacgacc tggcggcgac ggtggccaca gacctggaga gtggcggagc tggtgcagct |
| 241 | tgcagcagta acaacccggc cctcctagcc cggagggaga ccgaggagtt caacgacctc |
| 301 | ctggacctag actttatcct ttccaactcg ctaacccacc aggaatcggt ggccgccacc |
| 361 | gtgaccacct cggcgtcagc ttcatcctcg tcttccccgg cgagcagcgg ccctgccagc |
| 421 | gcgccctcca cctgcagctt cagctatccg atccgggccg ggggtgaccc gggcgtggct |
| 481 | gccagcaaca caggtggagg gctcctctac agccgagaat ctgcgccacc tcccacggcc |
| 541 | cccttcaacc tggcggacat caatgacgtg agcccctcgg gcggcttcgt ggctgagctc |
| 601 | ctgcggccgg agttggaccc agtatacatt ccgccacagc agcctcagcc gccaggtggc |
| 661 | gggctgatgg gcaagtttgt gctgaaggcg tctctgacca cccctggcag cgagtacagc |
| 721 | agcccttcgg tcatcagtgt tagcaaagga agcccagacg gcagccaccc cgtggtagtg |
| 781 | gcgccctaca gcggtggccc gccgcgcatg tgccccaaga ttaagcaaga ggcggtcccg |
| 841 | tcctgcacgg tcagccggtc cctagaggcc catttgagcg ctggacccca gctcagcaac |
| 901 | ggccaccggc ccaacacaca cgacttcccc ctggggcggc agctccccac caggactacc |
| 961 | cctacactga gtcccgagga actgctgaac agcagggact gtcaccctgg cctgcctctt |
| 1021 | cccccaggat tccatcccca tccggggccc aactaccctc ctttcctgcc agaccagatg |
| 1081 | cagtcacaag tcccctctct ccattatcaa gagctcatgc caccgggttc ctgcctgcca |
| 1141 | gaggagccca agccaaagag gggaagaagg tcgtggcccc ggaaaagaac agccacccac |
| 1201 | acttgtgact atgcaggctg tggcaaaacc tataccaaga gttctcatct oaaggcacac |
| 1261 | ctgcgaactc acacaggcga gaaaccttac cactgtgact gggacggctg tgggtggaaa |
| 1321 | ttcgcccgct ccgatgaact gaccaggcac taccgcaaac acacagggca ccggcccttt |
| 1381 | cagtgccaga agtgtgacag ggccttttcc aggtcggacc accttgcctt acacatgaag |
| 1441 | aggcactttt aa |
| Mouse C-Myc | |
| Primers | |
| (SEQ ID NO: 12) | |
| Xho-Myc-F: GATCC TCGAG CCCCT CAACG TGAAC TTCAC C | |
| (SEQ ID NO: 13) | |
| Eco-Myc-R: GATCG AATTC TTATG CACCA GAGTT TCGAA GCTG | |
| (SEQ ID NO: 14) | |
| Mouse C-Myc Sequence | |
| 1 | ctggatttcc tttgggcgtt ggaaaccccg cagacagcca cgacgatgcc cctcaacgtg |
| 61 | aacttcacca acaggaacta tgacctcgac tacgactccg tacagcccta tttcatctgc |
| 121 | gacgaggaag agaatttcta tcaccagcaa cagcagagcg agctgcagcc gcccgcgccc |
| 181 | agtgaggata tctggaagaa attcgagctg cttcccaccc cgcccctgtc cccgagccgc |
| 241 | cgctccgggc tctgctctcc atcctatgtt gcggtcgcta cgtccttctc cccaagggaa |
| 301 | gacgatgacg gcggcggtgg caacttctcc accgccgatc agctggagat gatgaccgag |
| 361 | ttacttggag gagacatggt gaaccagagc ttcatctgcg atcctgacga cgagaccttc |
| 421 | atcaagaaca tcatcatcca ggactgtatg tggagcggtt tctcagccgc tgccaagctg |
| 481 | gtctcggaga agctggcctc ctaccaggct gcgcgcaaag acagcaccag cctgagcccc |
| 541 | gcccgcgggc acagcgtctg ctccacctcc agcctgtacc tgcaggacct caccgccgcc |
| 601 | gcgtccgagt gcattgaccc ctcagtggtc tttccctacc cgctcaacga cagcagctcg |
| 661 | cccaaatcct gtacctcgtc cgattccacg gccttctctc cttcctcgga ctcgctgctg |
| 721 | tcctccgagt cctccccacg ggccagccct gagcccctag tgctgcatga ggagacaccg |
| 781 | cccaccacca gcagcgactc tgaagaagag caagaagatg aggaagaaat tgatgtggtg |
| 841 | tctgtggaga agaggcaaac ccctgccaag aggtcggagt cgggctcatc tccatcccga |
| 901 | ggccacagca aacctccgca cagcccactg gtcctcaaga ggtgccacgt ctccactcac |
| 961 | cagcacaact acgccgcacc cccctccaca aggaaggact atccagctgc caagagggcc |
| 1021 | aagttggaca gtggcagggt cctgaagcag atcagcaaca accgcaagtg ctccagcccc |
| 1081 | aggtcctcag acacggagga aaacgacaag aggcggacac acaacgtctt ggaacgtcag |
| 1141 | aggaggaacg agctgaagcg cagctttttt gccctgcgtg accagatccc tgaattggaa |
| 1201 | aacaacgaaa aggcccccaa ggtagtgatc ctcaaaaaag ccaccgccta catcctgtcc |
| 1261 | attcaagcag acgagcacaa gctcacctct gaaaaggact tattgaggaa acgacgagaa |
| 1321 | cagttgaaac acaaactcga acagcttcga aactctggtg cataa |
| Mouse Sall4 | |
| Primers | |
| (SEQ ID NO: 15) | |
| Sall4-F: CAGCG CCGCC GCGGT GGATC CACCA TGGCC ATGTC GAGGC GCAAG CAGGC G | |
| (SEQ ID NO: 16) | |
| Sall4-R: AAGCT TCGAA TTCAC CGCAT GCACT TAGCT GACAG CAATC TTATT TTCCT | |
| (SEQ ID NO: 17) | |
| Sall4 Sequence | |
| 1 | atgtcgaggc gcaagcaggc gaagccccag cacatcaact gggaggaggg ccagggcgag |
| 61 | cagcctcagc agctaccgag ccccgacctc gccgaggcgc tggcggcgga ggaacccggt |
| 121 | gctccagtga actcccctgg gaactgcgat gaagcctcag aggactccat accggtgaag |
| 181 | cggccccggc gggaggacac tcacatctgc aacaaatgct gtgccgagtt ctttagtctc |
| 241 | tctgaattca tggaacacaa gaaaagttgc actaaaaccc ctcctgtcct catcatgaat |
| 301 | gacagcgagg ggccagtgcc ttcagaggac ttttccagag ctgccctgag ccaccagctg |
| 361 | ggcagcccaa gcaataaaga cagtctccag gagaacggca gcagctcggg ggacttgaag |
| 421 | aagctgggca cggactccat cctgtacttg aagacagagg ctacccagcc atccacaccc |
| 481 | caggacataa gctatttacc caaaggcaaa gtagccaaca ccaatgtgac tctgcaggcg |
| 541 | ctccgcggca ccaaggtggc cgtgaaccaa cggggtgcag aggcacccat ggcgcccatg |
| 601 | cctgctgccc aaggcatccc ttgggtcctg gagcagatcc tgtgcctgca gcagcagcaa |
| 661 | ctccagcaaa tccagcttac ggaacagatt cgcgtccagg tgaacatgtg ggcagcgcac |
| 721 | gcgctccact ctggagtggc gggggccgac acgctgaagg ccttaagcag ccatgtgtct |
| 781 | cagcaagtgt ccgtgtccca gcaggtgtcg gctgccgtgg ccctgctcag ccagaaagcc |
| 841 | tcaaacccag ctctgtcgct cgatgccttg aaacaagcca agctacctca tgccagcgtc |
| 901 | ccctccgcag ccagcccgtt gtcctcgggg ttaacgtcct tcaccttgaa gcctgacggg |
| 961 | acacgggttc tccccaactt cgtgtctcgc cttcccagtg ccctgctacc tcagactccg |
| 1021 | ggctctgtgc tcctgcagag tcccttctcc gctgtgacgc tcgaccagtc caagaaagga |
| 1081 | aaggggaaac cccagaacct ctccgcctct gcctcggtgt tagatgtcaa ggccaaggac |
| 1141 | gaagtcgtcc tcggtaagca caagtgtagg tactgtccca aggttttcgg gacagatagc |
| 1201 | tcccttcaga ttcaccttcg ctcccacacc ggagagagac cttacgtgtg ccctatctgt |
| 1261 | ggtcaccgct tcaccaccaa gggcaatctc aaggtccact tacaacgaca ccctgaggtg |
| 1321 | aaggcaaacc cccagctgtt ggccgaattc caggacaaag gggcagtgag tgccgcttct |
| 1381 | cactatgcac tccctgtccc cgtccctgcc gatgaatcga gtctctctgt agacgccgag |
| 1441 | cctgtcccgg tcacgggaac cccttctcta gggctacctc aaaagctcac gtcagggcct |
| 1501 | aattccaggg acctcatggg tggctccttg cccaatgaca tgcagccagg gccttctcca |
| 1561 | gaaagtgagg cgggccttcc actccttggg gtggggatga tacataatcc cccaaaggct |
| 1621 | gggggcttcc agggcactgg ggccccagag tcagggtccg agaccctgaa attgcagcaa |
| 1681 | ctagtggaga acatagacaa ggccactact gaccccaacg agtgtctcat ttgtcatcgg |
| 1741 | gtcctcagct gtcagagttc cctgaagatg cattaccgta cccacacagg ggagagacca |
| 1001 | ttccagtgca agatctgtgg ccgggccttc tccaccaaag gcaacctgaa gacacacctt |
| 1061 | ggggttcacc gaaccaacac gaccgtaaag acccaacatt cgtgccccat ctgccagaag |
| 1921 | aaattcacca acgccgtcat gttacagcag catatccgga tgcacatggg tggccagatc |
| 1981 | cccaacaccc ctctgccaga gagtccctgt gacttcacgg ctcccgagcc cgtggccgtc |
| 2041 | agtgagaatg gcagtgccag cggggtctgc caggacgacg cagcagaagg gatggaagcc |
| 2101 | gaggaggtct gttctcagga tgttcccagt ggcccctcaa ctgtctctct gccggttccc |
| 2161 | agtgcccacc tggcatcgcc ctctctgggc ttctctgtgt tggcctccct ggatacgcag |
| 2221 | gggaaagggg ctcttccggc gctggccctg cagaggcaga gcagtcgaga aaacagctcc |
| 2281 | ctggagggcg gtgacactgg tccagccaat gactcttcct tgctcgtggg tgaccaggag |
| 2341 | tgtcagagcc gaagcccaga tgccacggag accatgtgct accaggcagt gtcacctgcc |
| 2401 | aatagccaag ccggaagtgt caagtcccgg tctcccgagg gtcacaaggc cgagggcgtg |
| 2461 | gagagctgcc gcgttgacac cgaaggtcgt accagcctcc ctccaacatt tatccgagca |
| 2521 | cagcccacct ttgtcaaagt tgaagtgcct ggcacctttg tgggaccccc cagcatgccc |
| 2581 | tcgggtatgc cgcctttgct agcatcgcag ccgcagccac gccgccaggc caagcagcac |
| 2641 | tgctgcacac ggtgtggaaa gaacttctcg tctgccagtg ccctgcagat ccacgagcga |
| 2701 | acacacacgg gagagaagcc tttcgtgtgt aacatatgcg ggcgggcctt caccacgaaa |
| 2761 | ggcaacctga aggtacacta catgactcat ggggccaaca ataactccgc ccgccgggga |
| 2821 | aggaagctgg ccatagagaa ccccatggcc gcgctgagtg ctgagggaaa gagagcgccc |
| 2881 | gaggtgtttt ccaaggagct cctgtccccc gcggtgagtg tggaccccgc ctcctggaac |
| 2941 | cagtacacca gcgtcctgaa tgggggtctg gccatgaaga ccaacgagat ctccgtgatc |
| 3001 | cagagcggag gcatccccac gctgcctgtg tcgctggggg ccagctctgt ggtgagcaat |
| 3061 | ggcacgattt ccaagcttga cggctctcag accggtgtga gcatgcccat gagcgggaac |
| 3121 | ggagaaaagc tcgctgttcc cgacggcatg gccaaacacc agttccctca cttcctggag |
| 3181 | gaaaataaga ttgctgtcag ctaa |

EGFP
Primers
(SEQ ID NO: 18)
Xho-EGFP-F: GATCC TCGAG ATGGT GAGCA AGGGC GAGGA GCTG
(SEQ ID NO: 19)
Eco-EGFP-R: GATCG AATTC TCAGT TATCT ACTTG TACAG CTCGT CCATG C
(SEQ ID NO: 20)
EGFP Sequence
ATGGTGAG CAAGGGCGAG GAGCTGTTCA CCGGGGTGGT GCCCATCCTG
GTCGAGCTGG ACGGCGACGT AAACGGCCAC AAGTTCAGCG TGTCCGGCGA GGGCGAGGGC
GATGCCACCT ACGGCAAGCT GACCCTGAAG TTCATCTGCA CCACCGGCAA GCTGCCCGTG
CCCTGGCCCA CCCTCGTGAC CACCCTGACC TACGGCGTGC AGTGCTTCAG CCGCTACCCC
GACCACATGA AGCAGCACGA CTTCTTCAAG TCCGCCATGC CCGAAGGCTA CGTCCAGGAG
CGCACCATCT TCTTCAAGGA CGACGGCAAC TACAAGACCC GCGCCGAGGT GAAGTTCGAG
GGCGACACCC TGGTGAACCG CATCGAGCTG AAGGGCATCG ACTTCAAGGA GGACGGCAAC
ATCCTGGGGC ACAAGCTGGA GTACAACTAC AACAGCCACA ACGTCTATAT CATGGCCGAC
AAGCAGAAGA ACGGCATCAA GGTGAACTTC AAGATCCGCC ACAACATCGA GGACGGCAGC
GTGCAGCTCG CCGACCACTA CCAGCAGAAC ACCCCCATCG GCGACGGCCC CGTGCTGCTG
CCCGACAACC ACTACCTGAG CACCCAGTCC GCCCTGAGCA AAGACCCCAA CGAGAAGCGC
GATCACATGG TCCTGCTGGA GTTCGTGACC GCCGCCGGGA TCACTCTCGG CATGGACGAG
CTGTACAAG TAG ATA ACT GA

The oligonucleotides and sequences for human somatic cell reprogramming factors are provided below. The respective sequences were verified once cloning of a respective factor was complete.

| Human C-Myc (accession no. BC000141) | |
|---|---|
| Primers | |
| (SEQ ID NO: 21) | |
| Xho-hsMyc-F: GATCC TCGAG ATGCC CCTCA ACGTT AGCTT CACCA AC | |
| (SEQ. ID NO: 22) | |
| Eco-hsMyc-R: GATCG AATTC TTACG CACAA GAGTT CCGTA GCTG | |
| (SEQ ID NO: 23) | |
| Human C-Myc Sequence | |
| 1 | ctggattttt ttcgggtagt ggaaaaccag cagcctcccg cgacgatgcc cctcaacgtt |
| 61 | agcttcacca acaggaacta tgacctcgac tacgactcgg tgcagccgta tttctactgc |
| 121 | gacgaggagg agaacttcta ccagcagcag cagcagagcg agctgcagcc cccggcgccc |
| 181 | agcgaggata tctggaagaa attcgagctg ctgcccaccc cgcccctgtc ccctagccgc |
| 241 | cgctccgggc tctgctcgcc ctcctacgtt gcggtcacac ccttctccct tcggggagac |
| 301 | aacgacggcg gtggcgggag cttctccacg gccgaccagc tggagatggt gaccgagctg |
| 361 | ctgggaggag acatggtgaa ccagagtttc atctgcgacc cggacgacga gaccttcatc |
| 421 | aaaaacatca tcatccagga ctgtatgtgg agcggcttct cggccgccgc caagctcgtc |
| 481 | tcagagaagc tggcctccta ccaggctgcg cgcaaagaca gcggcagccc gaaccccgcc |
| 541 | cgcggccaca gcgtctgctc cacctccagc ttgtacctgc aggatctgag cgccgccgcc |
| 601 | tcagagtgca tcgacccctc ggtggtcttc ccctaccctc tcaacgacag cagctcgccc |
| 661 | aagtcctgcg cctcgcaaga ctccagcgcc ttctctccgt cctcggattc tctgctctcc |
| 721 | tcgacggagt cctccccgca gggcagcccc gagcccctgg tgctccatga ggagacaccg |
| 781 | cccaccacca gcagcgactc tgaggaggaa caagaagatg aggaagaaat cgatgttgtt |
| 841 | tctgtggaaa agaggcaggc tcctggcaaa aggtcagagt ctggatcacc ttctgctgga |
| 901 | ggccacagca aacctcctca cagcccactg gtcctcaaga ggtgccacgt ctccacacat |
| 961 | cagcacaact acgcagcgcc tccctccact cggaaggact atcctgctgc caagagggtc |
| 1021 | aagttggaca gtgtcagagt cctgagacag atcagcaaca accgaaaatg caccagcccc |
| 1081 | aggtcctcgg acaccgagga gaatgtcaag aggcgaacac acaacgtctt ggagcgccag |
| 1141 | aggaggaacg agctaaaacg gagctttttt gccctgcgtg accagatccc ggagttggaa |
| 1201 | aacaatgaaa aggcccccaa ggtagttatc cttaaaaaag ccacagcata catcctgtcc |
| 1261 | gtccaagcag aggagcaaaa gctcatttct gaagaggact tgttgcggaa acgacgagaa |
| 1321 | cagttgaaac acaaacttga acagctacgg aactcttgtg cgtaa |
| Human Oct4 (acession no. BC117435) | |
| Primers | |
| (SEQ ID NO: 24) | |
| Nco-hsOct4-F: GATCC CATGG CGGGA CACCT GGCTT CGGAT TTC | |
| (SEQ ID NO: 25) | |
| Eco-hsOct4-R: GATCG AATTC TCAGT TTGAA TGCAT GGGAG AGC | |
| (SEQ ID NO: 26) | |
| Human Oct4 Sequence | |
| 1 | atggcgggac acctggcttc ggatttcgcc ttctcgcccc ctccaggtgg tggaggtgat |
| 61 | gggccagggg ggccggagcc gggctgggtt gatcctcgga cctggctaag cttccaaggc |
| 121 | cctcctggag ggccaggaat cgggccgggg gttgggccag gctctgaggt gtgggggatt |
| 181 | cccccatgcc ccccgccgta tgagttctgt ggggggatgg cgtactgtgg gccccaggtt |
| 241 | ggagtggggc tagtgcccca aggcggcttg gagacctctc agcctgaggg cgaagcagga |
| 301 | gtcggggtgg agagcaactc cgatggggcc tccccggagc cctgcaccgt cacccctggt |
| 361 | gccgtgaagc tggagaagga gaagctggag caaaacccgg aggagtccca ggacatcaaa |
| 421 | gctctgcaga aagaactcga gcaatttgcc aagctcctga agcagaagag gatcaccctg |
| 481 | ggatatacac aggccgatgt ggggctcacc ctgggggttc tatttgggaa ggtattcagc |
| 541 | caaacgacca tctgccgctt tgaggctctg cagcttagct tcaagaacat gtgtaagctg |
| 601 | cggcccttgc tgcagaagtg ggtggaggaa gctgacaaca atgaaaatct tcaggagata |
| 661 | tgcaaagcag aaaccctcgt gcaggcccga aagagaaagc gaaccagtat cgagaaccga |
| 721 | gtgagaggca acctggagaa tttgttcctg cagtgcccga aacccacact gcagcagatc |
| 781 | agccacatcg cccagcagct tgggctcgag aaggatgtgg tccgagtgtg gttctgtaac |
| 841 | cggcgccaga agggcaagcg atcaagcagc gactatgcac aacgagagga ttttgaggct |
| 901 | gctgggtctc ctttctcagg gggaccagtg tcctttcctc tggccccagg gccccatttt |
| 961 | ggtaccccag gctatgggag ccctcacttc actgcactgt actcctcggt ccctttccct |
| 1021 | gagggggaag cctttccccc tgtctccgtc accactctgg gctctcccat gcattcaaac |
| 1081 | tga |
| (SEQ ID NO: 27) | |
| Human Oct4 Protein sequence | |
| MAGHLASDFAFSPPPGGGGDGPGGPEPGWVDPRTWLSFQGPPGGPGIGPGVGPGSEVWGI | |
| PPCPPPYEFCGGMAYCGPQVGVGLVPQGGLETSQPEGEAGVGVESNSDGASPEPCTVTPG | |
| AVKLEFEKLEQNPEESQDIKALQKELEQFAKLLKQKRITLGYTQADVGLTLGVLFGKVFS | |
| QTTICRFEALQLSFKNMCKLRPLLQKWVEEADNNENLQEICKAETLVQARKRKRTSIENR | |
| VRGNLENLFLQCPKPTLQQISHIAQQLGLEKDVVRVWFCNRRQKGKRSSSDYAQREDFEA | |
| AGSPFSGGPVSFPLAPGPHFGTPGYGSPHFTALYSSVPFPEGEAFPPVSVTTLGSPMHSN | |
| Human Sox2 (accession no. BC013923) | |
| Primers | |
| (SEQ ID NO: 28) | |
| Xho-hsSox2-F: GATCC TCGAG ATGTA CAACA TGATG GAGAC GG | |
| (SEQ ID NO: 29) | |
| Eco-hsSox2-R: GATCG AATTC TCACA TGTGT GAGAG GGGCA GTGT | |
| (SEQ ID NO: 30) | |
| Human Sox2 Sequence | |
| 1 | atgtacaaca tgatggagac ggagctgaag ccgccgggcc cgcagcaaac ttcggggggc |
| 61 | ggcggcggca actccaccgc ggcggcggcc ggcggcaacc agaaaaacag cccggaccgc |
| 121 | gtcaagcggc ccatgaatgc cttcatggtg tggtcccgcg ggcagcggcg caagatggcc |
| 181 | caggagaacc ccaagatgca caactcggag atcagcaagc gcctgggcgc cgagtggaaa |
| 241 | cttttgtcgg agacggagaa gcggccgttc atcgacgagg ctaagcggct gcgagcgctg |
| 301 | cacatgaagg agcacccgga ttataaatac cggccccggc ggaaaaccaa gacgctcatg |
| 361 | aagaaggata agtacacgct gcccggcggg ctgctggccc ccggcggcaa tagcatggcg |
| 421 | agcggggtcg gggtgggcgc cggcctgggc gcgggcgtga accagcgcat ggacagttac |
| 481 | gcgcacatga acggctggag caacggcagc tacagcatga tgcaggacca gctgggctac |
| 541 | ccgcagcacc cgggcctcaa tgcgcacggc gcagcgcaga tgcagcccat gcaccgctac |
| 601 | gacgtgagcg ccctgcagta caactccatg accagctcgc agacctacat gaacggctcg |
| 661 | cccacctaca gcatgtccta ctcgcagcag ggcacccctg gcatggctct tggctccatg |
| 721 | ggttcggtgg tcaagtccga ggccagctcc agcccccctg tggttacctc ttcctcccac |
| 781 | tccagggcgc cctgccaggc cggggacctc cgggacatga tcagcatgta tctccccggc |
| 841 | gccgaggtgc cggaacccgc cgcccccagc agacttcaca tgtcccagca ctaccagagc |
| 901 | ggcccggtgc ccggcacggc cattaacggc acactgcccc tctcacacat gtga |
| (SEQ ID NO: 31) | |
| Human Sox2 protein sequence | |
| MYNMMETELKPPGPQQTSGGGGGNSTAAAAGGNQKNSPDRVKRPMNAFMVWSRGQRRKMA | |
| QENPKMHNSEISKRLGAEWKLLSETEKRPFIDEAKRLRALHMKEHPDYKYRPRRKTKTLM | |
| KKDKYTLPGGLLAPGGNSMASGVGVGAGLGAGVNQRMDSYAHMNGWSNGSYSMMQDQLGY | |
| PQHPGLNAHGAAQMQPMHRYDVSALQYNSMTSSQTYMNGSPTYSMSYSQQGTPGMALGSM | |
| GSVVKSEASSSPPVVTSSSHSRAPCQAGDLRDMISMYLPGAEVPEPAAPSRLHMSQHYQS | |
| GPVPGTAINGTLPLSHM* | |
| Human Klf4 (accession no. NM_004235) | |
| Primers | |
| (SEQ ID NO: 32) | |
| Xho-hsKlf4-F: GATCC TCGAG ATGGC TGTCA GGGAC GCGGT GCT | |
| (SEQ ID NO: 33) | |
| Eco-hsKlf4-R: GATCG AATTC TTAAA AATGC CTCTT CATGT GTAAG G | |
| (SEQ ID NO: 34) | |
| Human Klf4 Sequence | |
| 1 | atgaggcagc cacctggcga gtctgacatg gctgtcagcg acgcgctgct cccatctttc |
| 61 | tccacgttcg cgtctggccc ggcgggaagg gagaagacac tgcgtcaagc aggtgccccg |
| 121 | aataaccgct ggcgggagga gctctcccac atgaagcgac ttcccccagt gcttcccggc |
| 181 | cgcccctatg acctggcggc ggcgaccgtg gccacagacc tggagagcgg cggagccggt |
| 241 | gcggcttgcg gcggtagcaa cctggcgccc ctacctcgga gagagaccga ggagttcaac |
| 301 | gatctcctgg acctggactt tattctctcc aattcgctga cccatcctcc ggagtcagtg |
| 361 | gccgccaccg tgtcctcgtc agcgtcagcc tcctcttcgt cgtcgccgtc gagcagcggc |
| 421 | cctgccagcg cgccctccac ctgcagcttc acctatccga tccgggccgg gaacgacccg |
| 481 | ggcgtggcgc cgggcggcac gggcggaggc ctcctctatg gcagggagtc cgctccccct |
| 541 | ccgacggctc ccttcaacct ggcggacatc aacgacgtga gcccctcggg cggcttcgtg |
| 601 | gccgagctcc tgcggccaga attggacccg gtgtacattc cgccgcagca gccgcagccg |
| 661 | ccaggtggcg ggctgatggg caagttcgtg ctgaaggcgt cgctgagcgc ccctggcagc |
| 721 | gagtacggca gcccgtcggt catcagcgtc agcaaaggca gccctgacgg cagccacccg |
| 781 | gtggtggtgg cgccctacaa cggcgggccg ccgcgcacgt gccccaagat caagcaggag |
| 841 | gcggtctctt cgtgcaccca cttgggcgct ggaccccctc tcagcaatgg ccaccggccg |
| 901 | gctgcacacg acttccccct ggggcggcag ctccccagca ggactacccc gaccctgggt |
| 961 | cttgaggaag tgctgagcag cagggactgt caccctgccc tgccgcttcc tcccggcttc |
| 1021 | catccccacc cggggcccaa ttacccatcc ttcctgcccg atcagatgca gccgcaagtc |
| 1081 | ccgccgctcc attaccaaga gctcatgcca cccggttcct gcatgccaga ggagcccaag |
| 1141 | ccaaagacgg gaagacgatc gtggccccgg aaaaggaccg ccacccacac ttgtgattac |
| 1201 | gcgggctgcg gcaaaaccta cacaaagagt tcccatctca aggcacacct gcgaacccac |
| 1261 | acaggtgaga aaccttacca ctgtgactgg gacggctgtg gatggaaatt cgcccgctca |
| 1321 | gatgaactga ccaggcacta ccgtaaacac acggggcacc gcccgttcca gtgccaaaaa |
| 1381 | tgcgaccgag cattttccag gtcggaccac ctcgccttac acatgaagag gcatttttaa |
| (SEQ ID NO: 35) | |
| Klf4 protein sequence | |
| MAVSDALLPSFSTFASGPAGREKTLRQAGAPNNRWREELSHMKRLPPVLPGRPYDLAAAT | |
| VATDLESGGAGAAGGGSNLAPLPRRETEEFNDLLDLDFILSNSLTHPPESVAATVSSSAS | |
| ASSSSSPSSSGPASAPSTCSFTYPIRAGNDPGVAPGGTGGGLLYGRESAPPPTAPFNLAD | |
| INDVSPSGGFVAELLRPELDPVYIPPQOPQPPGGGLMGKFVLKASESAPGSEYGSPSVIS | |
| VSKGSPDGSHPVVVAPYNGGPPRTCPKIKQEAVSSCTHLGAGPPLSNGHRPAAHDFPLGR | |
| QLPSRTTPTLGLEEVLSSRDCHPALPLPPGFHPHPGPNYPSFLPDQMQPQVPPLHYQELM | |
| PPGSCMPEEPKPKRGRRSWPRKRTATHTCDYAGCGKTYTKSSHLKAHLRTHTGEKPYHCD | |
| WDGCGWKFARSDELTRHYRKHTGHRPFQCQKCDRAFSRSDHLALHMKRHF* | |
| Human Sall4A (hSall4A) (accession no. AY172738) | |
| Primers | |
| (SEQ ID NO: 36) | |
| Kpn-hsSall4-F: GATCG GTACC ATGTC GAGGC GCAAG CAGGC GAAAC | |
| (SEQ ID NO: 37) | |
| Eco-hsSall4-R: GATCG AATTC TTAGC TGACC GCAAT CTTGT TTTC | |
| (SEQ ID NO: 38) | |
| hSall4A Sequence | |
| 1 | atgtcgaggc gcaagcaggc gaaaccccag cacatcaact cggaggagga ccagggcgag |
| 61 | cagcagccgc agcagcagac cccggagttt gcagatgcgg ccccagcggc gcccgcggcg |
| 121 | ggggagctgg gtgctccagt gaaccaccca gggaatgacg aggtggcgag tgaggatgaa |
| 181 | gccacagtaa agcggcttcg tcgggaggag acgcacgtct gtgagaaatg ctgtgaggag |
| 241 | ttcttcagca tctctgagtt cctggaacat aagaaaaatt gcactaaaaa tccacctgtc |
| 301 | ctcatcatga atgacagcga ggggcctgtg ccttcagaag acttctccgg agctgtactg |
| 361 | agccaccagc ccaccagtcc cggcagtaag gactgtcaca gggagaatgg cggcagctca |
| 421 | gaggacatga aggagaagcc ggatgcggag tctgtggtgt acctaaagac agagacagcc |
| 481 | ctgccaccca ccccccagga cataagctat ttagccaaag gcaaagtggc caacactaat |
| 541 | gtgaccttgc aggcactacg gggcaccaag gtggcggtga atcagcggag cgcggatgca |
| 601 | ctccctgccc ccgtgcctgg tgccaacagc atcccgtggg tcctcgagca gatcttgtgt |
| 661 | ctgcagcagc agcagctaca gcagatccag ctcaccgagc agatccgcat ccaggtgaac |
| 721 | atgtgggcct cccacgccct ccactcaagc ggggcagggg ccgacactct gaagaccttg |
| 781 | ggcagccaca tgtctcagca ggtttctgca gctgtggctt tgctcagcca gaaagctgga |
| 841 | agccaaggtc tgtctctgga tgccttgaaa caagccaagc tacctcacgc caacatccct |
| 901 | tctgccacca gctccctgtc cccagggctg gcacccttca ctctgaagcc ggatgggacc |
| 961 | cgggtgctcc cgaacgtcat gtcccgcctc ccgagcgctt tgcttcctca ggccccgggc |
| 1021 | tcggtgctct tccagagccc tttctccact gtggcgctag acacatccaa gaaagggaag |
| 1081 | gggaagccac cgaacatctc cgcggtggat gtcaaaccca aagacgaggc ggccctctac |
| 1141 | aagcaaaagt gtaagtactg tagcaaggtt tttgggactg atagctcctt gcagatccac |
| 1201 | ctccgctccc acactggaga gagacccttc gtgtgctctg tctgtggtca tcgcttcacc |
| 1261 | accaagggca acctcaaggt gcactttcac cgacatcccc aggtgaaggc aaacccccag |
| 1321 | ctgtttgccg agttccagga caaagtggcg gccggcaatg gcatccccta tgcactctct |
| 1381 | gtacctgacc ccatagatga accgagtctt tctttagaca gcaaacctgt ccttgtaacc |
| 1441 | acctctgtag ggctacctca gaatctttct tcggggacta atcccaagga cctcacgggt |
| 1501 | ggctccttgc ccggtgacct gcagcctggg ccttctccag aaagtgaggg tggacccaca |
| 1561 | ctccctgggg tgggaccaaa ctataattcc ccaagggctg gtggcttcca agggagtggg |
| 1621 | acccctgagc cagggtcaga gaccctgaaa ttgcagcagt tggtggagaa cattgacaag |
| 1681 | gccaccactg atcccaacga atgtctcatt tgccaccgag tcttaagctg tcagagctcc |
| 1741 | ctcaagatgc attatcgcac ccacaccggg gagagaccgt tccagtgtaa gatctgtggc |
| 1801 | cgagcctttt ctaccaaagg taacctgaag acacaccttg gggttcaccg aaccaacaca |
| 1861 | tccattaaga cgcagcattc gtgccccatc tgccagaaga agttcactaa tgccgtgatg |
| 1921 | ctgcagcaac atattcggat gcacatgggc ggtcagattc ccaacacgcc cctgccagag |
| 1981 | aatccctgtg actttacggg ttctgagcca atgaccgtgg gtgagaacgg cagcaccggc |
| 2041 | gctatctgcc atgatgatgt catcgaaagc atcgatgtag aggaagtcag ctcccaggag |
| 2101 | gctcccagca gctcctccaa ggtccccacg cctcttccca gcatccactc ggcatcaccc |
| 2161 | acgctagggt ttgccatgat ggcttcctta gatgccccag ggaaagtggg tcctgcccct |
| 2221 | tttaacctgc agcgccaggg cagcagagaa aacggttccg tggagagcga tggcttgacc |
| 2281 | aacgactcat cctcgctgat gggagaccag gagtatcaga gccgaagccc agatatcctg |
| 2341 | gaaaccacat ccttccaggc actctccccg gccaatagtc aagccgaaag catcaagtca |
| 2401 | aagtctcccg atgctgggag caaagcagag agctccgaga acagccgcac tgagatggaa |
| 2461 | ggtcggagca gtctcccttc cacgtttatc cgagccccgc cgacctatgt caaggttgaa |
| 2521 | gttcctggca catttgtggg accctcgaca ttgtccccag ggatgacccc tttgttagca |
| 2581 | gcccagccac gccgacaggc caagcaacat ggctgcacac ggtgtgggaa gaacttctcg |
| 2641 | tctgctagcg ctcttcagat ccacgagcgg actcacactg gagagaagcc ttttgtgtgc |
| 2701 | aacatttgtg ggcgagcttt taccaccaaa ggcaacttaa aggttcacta catgacacac |
| 2761 | ggggcgaaca ataactcagc ccgccgtgga aggaagttgg ccatcgagaa caccatggct |
| 2821 | ctgttaggta cggacggaaa aagagtctca gaaatctttc ccaaggaaat cctggcccct |
| 2881 | tcagtgaatg tggaccctgt tgtgtggaac cagtacacca gcatgctcaa tggcggtctg |
| 2941 | gccgtgaaga ccaatgagat ctctgtgatc cagagtgggg gggttcctac cctcccggtt |
| 3001 | tccttggggg ccacctccgt tgtgaataac gccactgtct ccaagatgga tggctcccag |
| 3061 | tcgggtatca gtgcagatgt ggaaaaacca agtgctactg acggcgttcc caaacaccag |
| 3121 | tttcctcact tcctggaaga aaacaagatt gcggtcagct aa |
| (SEQ ID NO: 39) | |
| hSall4B (accession no. AY170621) | |
| 1 | atgtcgaggc gcaagcaggc gaaaccccag cacatcaact cggaggagga ccagggcgag |
| 61 | cagcagccgc agcagcagac cccggagttt gcagatgcgg ccccagcggc gcccgcggcg |
| 121 | ggggagctgg gtgctccagt gaaccaccca gggaatgacg aggtggcgag tgaggatgaa |
| 181 | gccacagtaa agcggattcg tcgggaggag acgcacgtct gtgagaaatg ctgtgcggag |
| 241 | ttcttcagca tctctgagtt cctggaacat aagaaaaatt gcactaaaaa tccacctgtc |
| 301 | ctcatcatga atgacagcga ggggcctgtg ccttcanaag acttctccgg agctgtactg |
| 361 | agccaccagc ccaccagtcc cggcagtgag gactgtcaca gggagaatgg cggcagctca |
| 421 | naggacataa aggagaagcc ggatgcggag tctgtggtgt acctaaagac agagacagcc |
| 481 | ctgccaccca ccccccagga cataagctat ttagccaaag gcaaagtggc caacactaac |
| 541 | gtgaccttgc aggcactacg gggcaccaag gtggcggtga atcagcggag cgcggatgca |
| 601 | ctccctgccc ccgtgcctgg tgccaacagc atcccgtggg tcctcgagca gatcttgtgt |
| 661 | ctgcagcagc agcagctaca gcagatccag ctcaccgagc agatccgcat ccaggtgaac |
| 721 | atgtgggcct cccacgccct ccactcaagc ggggcagggg ccgacactct gaagaccttg |
| 781 | ggcagccaca tgtctcagca ggtttctgca gctgtggctt tgctcagcca gaaagctgga |
| 841 | agccaaggtc tgtctctgga tgccttgaaa caagccaagc tacctcacgc caacatccct |
| 901 | tctgccacca gctccctgtc cccagggctg gcacccttca ctctgaagcc ggatgggacc |
| 961 | cgggtgctcc cgaacgtcat gtcccgcctc ccgagcgctt tgcttcctca ggccccgggc |
| 1021 | tcggtgctct tccagagccc tttctccact gtggcgctag acacatccaa gaaagggaag |
| 1081 | gggaagccac cgaacatctc cgcggtggat gtcaaaccca aagacgaggc ggccctctac |
| 1141 | aagcacaagt gtcggagcag tctcccttcc acgtttatcc gagccccgcc gacctatgtc |
| 1201 | aaggttgaag ttcctggcac atttgtggga ccctcgacat tgtccccagg gatgacccct |
| 1261 | ttgttagcag cccagccacg cggacaggcc aagcaacatg gctgcacacg gtgtggnaag |
| 1321 | aacttntcgt ntgntagcgc tcttcagatc cacgagcgga ctcacantgg agagaagcct |
| 1381 | tttgtgtgca acatttgtgg gcgagctttt accaccaaag gcaacttaaa ggttcactac |
| 1441 | atgacacacg gggcgaacaa taactcagcc cgccgtggaa ggaagttggc catcgagaac |
| 1501 | accatggctc tgttaggtac ggacggaaaa agagtctcag aaatctttcc caaggaaatc |
| 1561 | ctggcccctt cagtgaatgt ggaccctgtt gtgtggaacc agtacaccag catgctcaat |
| 1621 | ggcggtctgg ccgtgaagac caatgagatc tctgtgatcc agagtggggg ggttcctacc |
| 1681 | ctcccggttt ccttgggggc cacctccgtt gtgaataacg ccactgtctc caagatggat |
| 1741 | ggctcccagt cgggtatcag tgcagatgtg gaaaaaccaa gtgctactga cggcgttccc |
| 1801 | aaacnccagt ttcctcactt cctggaagaa aacaagantg cggtcagcta a |
| h-UTF1 (accession no. NM_03577.2) | |
| Primers | |
| (SEQ ID NO: 40) | |
| h-UTf1-F: GATCC TCGAG ATGCT GCTCC GGCCC CGCAG GCCGC | |
| (SEQ ID NO: 41) | |
| h-UTF1-R: GATCG AATTC TCACT GGCAC GGGTC CCTGA GGACC C | |
| (SEQ ID NO: 42) | |
| Human UTF1 Sequence | |
| 1 | atgctgctcc ggccccgcag gccgcccccg ctcgcgcccc ccgcgccgcc ctcgcccgcc |
| 61 | agccccgacc ccgagccgcg gacacccgga gacgccccgg ggaccccgcc ccggaggccc |
| 121 | gcctcgccca gcgcgctggg ggaactcggg ttgccggtgt ccccgggctc ggcgcagcgc |
| 181 | acgccctgga gcgcccggga gacggagctg ctgctgggga cgctgctgca accggccgtg |
| 241 | tggcgcgcgc tgctcctgga ccgccgccag gccctgccca cctaccgccg cgtgtcggcc |
| 301 | gcgctggccc agcagcaggt gcgccgcacc cccgcgcagt gccgccgccg ctacaagttc |
| 361 | cttaaagaca agtttcgcga ggcgcacggc cagccgcccg ggcccttcga cgagcagatc |
| 421 | cggaagctca tggggctgct gggcgacaac gggcgcaaac ggcctcgccg ccgctccccg |
| 481 | gggtccgggc gcccccagcg cgcccgccgc ccggtcccca acgcgcacgc gccggctccc |
| 541 | agcgaaccag acgccacccc gctgcccacc gcccgcgacc gcgacgcgga ccccacctgg |
| 601 | acgctccgct tcagcccgtc cccaccgaag tctgcggacg cctcccccgc ccccggctcc |
| 661 | ccgccagctc ccgccccgac cgccctcgcc acctgcatcc ccgaggaccg cgcgcccgtc |
| 721 | cgcggccccg ggtccccgcc gccacccccg gcccgcgaag accccgactc gccgcccggc |
| 781 | cgccccgagg actgcgcgcc ccctccggcc gcgcccccgt cgctgaacac cgccctgctg |
| 841 | cagaccctgg ggcacctggg cgacatcgcg aacatcctgg gcccgctgcg cgaccagctg |
| 901 | ctgaccttga accagcacgt ggagcagctg cgcggcgcct tcgaccagac agtgtccctg |
| 961 | gccgtgggct tcattctggg cagcgcggcc gccgagcgag gggtcctcag ggacccgtgc |
| 1021 | cagtga |

To optimize the expression of mammalian stem cell factors in bacteria, the above genes were codon-optimized, synthesized and cloned into pTATHA. All clones were sequence-verified. The codon optimized sequences are provided below.

Mouse myc_codon_optimized sequence
(SEQ ID NO: 43)
gatcctcgagCCACTCAACGTTAATTTTACCAATCGTAATTATGACCTCGACTATGACTCAGTCCAGCCTT
ACTTCATCTGTGATGAGGAAGAAAACTTCTACCACCAACAGCAGCAAAGCGAACTGCAACCGCCCGCGCCT
AGTGAAGATATTTGGAAAAAATTTGAATTACTGCCGACCCCCCCCCTGTCCCCGTCCCGTCGTTCAGGACT
TTGTAGCCCGTCTTATGTGGCCGTCGCGACTAGCTTTTCACCTCGTGAGGACGATGATGGAGGCGGTGGCA
ACTTTTCGACCGCAGATCAACTCGAAATGATGACAGAACTTTTAGGCGGAGATATGGTAAATCAGTCTTTC
ATTTGTGACCCTGATGACGAAACCTTTATCAAAAACATTATTATTCAAGATTGCATGTGGTCTGGCTTTAG
CGCCGCCGCGAAACTTGTAAGCGAAAAATTAGCCTCATATCAAGCAGCACGCAAAGATTCTACCTCACTCA
GCCCTGCCCGCGGACACTCTGTATGTTCCACGTCTTCTCTGTACCTCCAAGACCTTACTGCCGCAGCCAGC
GAATGTATTGACCCGAGTGTTGTGTTTCCATATCCACTGAATGATTCCTCTAGTCCCAAATCTTGTACCTC
ATCCGACAGCACCGCATTCTCGCCGAGCTCAGACTCACTGTTATCCTCCGAAAGCAGCCCTCGCGCCTCCC
CCGAACCATTGGTTTTACACGAAGAAACACCACCAACCACTTCATCCGACTCTGAAGAAGAACAAGAAGAC
GAAGAAGAAATTGATGTAGTCAGTGTGGAAAAGCGTCAAACCCCGGCGAAACGTAGCGAATCTGGTTCCTC
TCCCTCGCGCGGACATTCTAAACCCCCACATAGCCCCCTCGTTTTAAAACGTTGTCACGTTTCAACTCACC
AGCATAATTATGCAGCACCACCATCTACCCGCAAAGACTATCCAGCAGCAAAACGCGCCAAACTCGATTCC
GGCCGCGTCCTGAAGCAAATTTCTAACAATCGCAAATGTTCCTCACCCCGTTCATCCGATACCGAAGAAAA
TGATAAACGCCGTACCCATAACGTTCTGGAACGCCAACGCCGTAACGAACTGAAACGTTCCTTTTTCGCAT
TGCGCGATCAGATCCCGGAGCTCGAAAATAATGAAAAAGCACCTAAAGTAGTTATCCTGAAAAAAGCAACC
GCATATATTCTGAGCATTCAAGCCGACGAACACAAATTAACATCCGAAAAAGACTTATTACGTAAACGTCG
CGAACAACTGAAACATAAACTGGAACAATTACGCAACTCCGGAGCGTAAgaattcgatc
Mouse myc protein sequence
(SEQ ID NO: 44)
PLNVNFTNRNYDLDYDSVQPYFICDEEENFYHQQQQSELQPPAPSEDIWKKFELLPTPPL
SPSRRSGLCSPSYVAVATSFSPREDDDGGGGNFSTADQLEMMTELLGGDMVNQSFICDPD
DETFIKNIIIQDCMWSGFSAAAKLVSEKLASYQAARKDSTSLSPARGHSVCSTSSLYLQD
LTAAASECIDPSVVFPYPLNDSSSPKSCTSSDSTAFSPSSDSLLSSESSPRASPEPLVLH
EETPPTTSSDSEEEQEDEEEIDVVSVEKRQTPAKRSESGSSPSRGHSKPPHSPLVLKRCH
VSTHQHNYAAPPSTRKDYPAAKRAKLDSGRVLKQISNNRKCSSPRSSDTEENDKRRTHNV
LERQRRNELKRSFFALRDQIPELENNEKAPKVVILKKATAYILSIQADEHKLTSEKDLLR
KRREQLKHKLEQLRNSGA-
Human myc codon optimized DNA sequence
(SEQ ID NO: 45)
gatcctccagATGCCCCTTAATGTCTCATTTACGAACCGTAACTACGATCTTGATTACGACAGCGTTCAAC
CTTACTTTTACTGCGATGAAGAAGAAAATTTCTATCAGCAACAACAGCAAAGCGAACTGCAACCCCCGGCC
CCTTCAGAGGATATCTGGAAAAAATTCGAACTTTTGCCAACCCCGCCCCTGTCACCTTCTCGCCGCTCTGG
TTTATGCTCCCCGTCCTATGTAGCCGTCACTCCATTTTCCTTACGTGGTGATAACGACGGTGGTGGCGGTA
GCTTTTCAACCGCCGATCAGTTAGAAATGGTTACCGAACTCTTAGGCGGCGATATGGTTAATCAGTCTTTC
ATTTGTGACCCAGATGACGAAACCTTTATTAAAAACATTATCATTCAAGACTGCATGTGGTCTGGTTTCTC
AGCCGCCGCAAAACTTGTGTCTGAAAAACTTGCATCCTACCAAGCTGCCCGCAAAGATTCCGGCTCCCCAA
ACCCCGCTCGTGGCCATTCCGTGTGTAGCACCTCGTCCCTTTATTTGCAGGACTTATCAGCAGCAGCATCT
GAATGTATCGATCCGTCCGTTGTCTTCCCATACCCGTTGAATGACTCAAGCTCTCCAAAATCCTGCGCCTC
CCAAGATTCCTCCGCTTTTAGCCCCTCCTCCGATAGTCTCCTTTCTTCCACCGAGAGTTCCCCACAGGGAT
CCCCAGAACCGTTAGTTTTGCACGAAGAAACGCCTCCAACCACCTCAAGCGATAGCGAAGAAGAACAAGAA
GATGAAGAAGAAATTGATGTTGTTTCCGTTGAAAAACGCCAAGCCCCAGGTAAACGCTCCGAATCCGGCTC
TCCATCCGCTGGCGGCCACTCTAAACCACCTCATAGCCCGTTAGTACTCAAACGCTGCCATGTCTCTACCC
ATCAACATAATTATGCCGCACCTCCAAGTACGCGCAAAGACTACCCAGCAGCCAAACGCGTGAAACTGGAT
AGTGTCCGTGTCCTCCGTCAAATTAGCAATAATCGTAAATGCACTTCTCCCCGGTCCTCAGATACTGAAGA
AAACGTAAAACGCCGTACTCATAACGTCTTAGAACGTCAGCGCCGTAACGAACTGAAACGCTCATTTTTTG
CGCTTCGTGATCAAATCCCCGAATTAGAAAATAATGAAAAAGCGCCTAAAGTTGTTATCCTGAAAAAAGCC
ACAGCCTATATCTTATCCGTACAAGCCGAAGAACAAAAACTTATCTCTGAAGAAGATCTGCTCCGCAAACG
CCGTGAACAATTAAAACATAAACTGGAACAATTACGTAATAGCTGCGCCTAAgaattcgatc
Human Myc protein sequence
(SEQ ID NO: 46)
MPLNVSFTNRNYDLDYDSVQPYFYCDEEENFYQQQQQSELQPPAPSEDIWKEFELLPTPP
LSPSPRSGLCSPSYVAVTPFSLRGDNDGGGGSFSTADQLEMVTELLGGDMVNQSFICDPD
DETFIKNIIIQDCMWSGFSAAAKLVSEKLASYQAARKDSGSPNPARGHSVCSTSSLYLQD
LSAAASECIDPSVVFPYPLNDSSSPKSCASQDSSAFSPSSDSLLSSTESSPQGSPEPLVL
HEETPPTTSSDSEEEQEDEEEIDVVSVEKRQAPGKRSESGSPSAGGHSKPPHSPLVLKRC
HVSTHQHNYAAPPSTRKDYPAAKRVKLDSVRVLRQISNNRKCTSPRSSDTEENVKRRTHN
VLERQRRNELKRSFFALRDQIPELENNEKAPKVVILKKATAYILSVQAEEQKLISEEDLL
RKRREQLKHKLEQLRNSCA
Human oct4 codon optimized DNA sequence
(SEQ ID NO: 47)
gatcccatggATGGCTGGTCATCTTGCAAGTGATTTCGCCTTTTCACCTCCCCCAGGTGGCGGCGGTGACG
GTCCGGGCGGTCCAGAACCAGGTTGGGTTGATCCACGCACGTGGTTAAGTTTTCAAGGICCTCCAGGTGGT
CCAGGAATTGGTCCCGGTGTTGGCCCCGGCAGTGAAGTGTGGGGCATCCCCCCGTGTCCTCCCCCCTATGA
ATTTTGCGGTGGCATGGCGTATTGCGGTCCTCAAGTTGGTGTTGGTTTGGTCCCACAAGGTGGTCTCGAAA
CCTCACAACCCGAAGGAGAAGCTGGCGTGGGTGTAGAATCAAACAGCGATGGCGCCTCAGCTGAACCATGC
ACTGTCACTCCTGGCGCGGTTAAATTGGAAAAAGAAAAATTAGAGCAGAACCCAGAAGAATCCCAAGATAT
CAAAGCCCTTCAGAAAGAATTAGAACAATTTGCCAAACTCTTGAAACAAAAACGTATCACTCTCGGATATA
CGCAAGCCGATGTTGGCCTGACCCTCGGTGTATTATTCGGGAAAGTATTTTCACAGACAACAATCTGCCGT
TTTGAAGCACTGCAACTGTCTTTTAAAAACATGTGCAAATTACGCCCCCTGCTGCAGAAATGGGTCGAAGA
AGCAGATAACAATGAAAACTTACAGGAAATTTGCAAGGCCGAAACCTTAGTTCAAGCTCGCAAACGTAAAC
GCACCAGCATTGAAAATCGTGTACGTGGTAATCTCGAAAATTTATTCTTACAGTGTCCTAAACCAACTTTA
CAGCAAATCAGCCATATCGCTCAGCAACTCGGTCTTGAGAAAGACGTCGTTCGGGTTTGGTTTTGTAATCG
TCGTCAAAAAGGTAAACGCTCGTCATCCGACTACGCCCAACGGGAAGATTTTGAAGCTGCAGGTAGTCCCT
TTAGTGGCGGCCCCGTTTCGTTCCCCCTCGCTCCAGGCCCACATTTTGGTACCCCAGGTTACGGTAGTCCT
CATTTTACAGCATTATATTCATCCGTTCCGTTTCCCGAAGGCGAGGCATTCCCTCCAGTATCGGTTACTAC
TCTCGGCTCACCTATC3CACTCCAATTAAgaattcgatc
Human Oct4 codon optimized protein sequence
(SEQ ID NO: 48)
MAGHLASDFAFSPPPGGGGDGPGGPEPGWVDPRTWLSFQGPPGGPGIGPGVGPGSEVWGI
PPCPPPYEFCGGMAYCGPQVGVGLVPQGGLETSQPEGEAGVGVESNSDGASPEPCTVTPG
AVKLEKEKLEQNPEESQDIKALQKELEQFAKLLKQKRITLGYTQADVGLTLGVLFGKVFS
QTTICRFEALQLSFKNMCKLRPLLQKWVEEADNNENLQEICKAETLVQARKRKRTSIENR
VRGNLENLFLQCPKPTLQQISHIAQQLGLEKDVVRVWFCNRRQKGKRSSSDYAQREDFEA
AGSPFSGGPVSFPLAPGPHFGTPGYGSPHFTALYSSVPFPEGEAFPPVSVTTLGSPMHSN
Human Sox2 codon optimized DNA sequence
(SEQ ID NO: 49)
gatcctcgagATGTACAACATGATGGAAACAGAACTCAAACCTCCAGGCCCTCAACAAACTTCCGGTGGTG
GCGGCGGCAACTCAACTGCAGCAGCAGCAGGTGGTAATCAGAAAAATAGCCCGGATCGTGTTAAACGCCCG
ATGAACGCATTTATGGTATGGTCCCGCGGTCAACGTCGGAAAATGGCTCAAGAAAACCCTAAAATGCATAA
CAGCGAAATTTCTAAACGTTTAGGTGCTGAATGGAAACTCTTATCTGAAACCGAAAAACGTCCGTTTATTG
ATGAAGCCAAACGCTTGCGCGCGCTCCACATGAAAGAACATCCCGATTATAAATACCGTCCTCGTCGTAAA
ACCAAAACGTTAATGAAAAAAGATAAATACACTCTTCCAGGTGGTCTCTTAGCTCCAGGCGGTAACTCTAT
GGCGTCAGGGGTCGGGGTCGGTGCTGGACTGGGGGCCGGAGTTAATCAGCGTATGGACTCTTATGCCCACA
TGAACGGTTGGTCAAATGGCAGCTACAGCATGATGCAAGATCAGCTTGGTTATCCTCAACATCCCGGTTTG
AACGCTCATGGCGCAGCTCAAATGCAACCGATGCACCGTTACGACGTATCCGCATTACAGTATAACAGTAT
GACTAGCTCGCAAACTTACATGAATGGATCACCGACCTACAGTATGAGTTATTCACAACAAGGCACCCCCG
GCATGGCCTTAGGCTCAATGGGCTCCGTCGTCAAATCCGAAGCATCCTCTTCCCCACCAGTCGTTACGTCC
TCCTCACACTCTCGTGCACCTTGTCAAGCTGGAGATTTACGCGATATGATCTCAATGTATCTCCCCGGCGC
AGAAGTACCAGAACCAGCCGCTCCTTCACGTCTTCACATGTCTCAGCATTATCAATCTGGCCCTGTTCCAG
GTACCGCAATTAACGGCACATTACCATTATCTCACATGTAAgaattcgatc
Human Sox2 codon optimized protein sequence
(SEQ ID NO: 50)
MYNMMETELKPPGPQQTSGGGGGNSTAAAAGGNQKNSPDRVKRPMNAFMVWSRGQRRKMA
QENPKMHNSEISKRLGAEWKLLSETEKRPFIDEAKRLRALHMKEHPDYKYRPRRKTKTLM
KKDKYTLPGGLLAPGGNSMASGVGVGAGLGAGVNQRMDSYAHMNGWSNGSYSMMQDQLGY
PQHPGLNAHGAAQMQPMHRYDVSALQYNSMTSSQTYMNGSPTYSMSYSQQGTPGMALGSM
GSVVKSEASSSPPVVTSSSHSRAPCQAGDLRDMISMYLPGAEVPEPAAPSRLHMSQHYQS
Human klf4 codon optimized DNA sequence
(SEQ ID NO: 51)
gatcctcgagATGGCCGTCTCCGACGCACTGTTGCCTAGCTTCAGCACCTTTGCTTCAGGTCCCGCAGGCC
GCGAAAAAACACTCCGCCAAGCAGGCGCCCCCAATAACCGTTGGCGCGAAGAACTTTCACATATGAAACGT
CTGCCCCCAGTGTTGCCGGGTCGCCCTTATGATTTAGCTGCAGCGACCGTGGCCACCGACCTCGAATCGGG
TGGAGCAGGCGCAGCCTGTGGTGGCAGTAATTTAGCCCCTCTTCCCCGTCGCGAAACTGAAGAATTTAATG
ATCTGTTAGACCTCGACTTTATTTTATCCAACTCTCTCACCCATCCACCAGAATCAGTCGCCGCAACTGTT
TCGTCCTCCGCATCAGCTTCATCGTCTAGCTCTCCGTCGTCAAGCGGCCCTGCATCGGCCCCATCTACATG
CTCTTTTACATACCCCATCCGCGCTGGTAACGATCCGGGTGTTGCCCCAGGAGGTACCGGAGGAGGCTTAC
TGTATGGTCGCGAATCAGCCCCTCCACCGACAGCCCCGTTCAACCTTGCCGATATTAATGACGTGTCCCCT
AGTGGTGGCTTTGTGGCCGAATTGCTGCGTCCAGAACTTGACCCCGTTTATATCCCGCCTCAACAACCTCA
GCCCCCTGGCGGTGGCCTCATGGGTAAATTTGTCTTAAAAGCAAGCTTGTCCGCACCTGGTTCCGAATATG
GTAGTCCTTCCGTTATCTCTGTTTCCAAGGGTTCTCCTGATGGCTCCCATCCAGTTGTAGTTGCACCTTAT
AATGGCGGTCCCCCACGTACCTGTCCTAAAATCAAACAGGAAGCTGTTTCCTCCTGCACACATTTAGGTGC
CGGCCCTCCTCTGAGCAACGGCCATCGCCCAGCGGCCCACGATTTCCCTTTAGGTCGTCAACTTCCATCCC
GTACGACACCAACCTTAGGCTTAGAAGAAGTCCTGTCCTCTCGTGACTGCCATCCTGCTTTACCTCTGCCT
CCAGGTTTTCATCCACATCCAGGCCCGAATTACCCTTCCTTCTTACCAGATCAAATGCAACCACAAGTCCC
CCCCTTACACTACCAAGAACTGATGCCACCGGGCTCCTGCATGCCAGAAGAACCAAAACCGAAACGCGGCC
GCCGTTCCTGGCCCCGCAAACGTACCGCCACCCACACCTGTGACTATGCTGGTTGCGGCAAAACATACACT
AAAAGTTCACACCTTAAAGCACATCTTCGTACGCATACTGGCGAAAAACCTTATCACTGCGATTGGGATGG
CTGTGGTTGGAAATTCGCACGCTCCGATGAGTTAACCCGTCATTATCGCAAACATACTGGACATCGCCCAT
TCCAATGCCAAAAATGCGATCGCGCGTTTTCCCGTTCAGACCATTTAGCCTTACACATGAAACGCCACTTT
TAAgaattcgatc
Human klf3 codon optimized protein sequence
(SEQ ID NO: 52)
MAVSDALLPSFSTFASGPAGREKTLRQAGAPNNRWREELSHMKRLPPVLPGRPYDLAAAT
VATDLESGGAGAACGGSNLAPLPRRETEEFNDLLDLDFILSNSLTHPPESVAATVSSSAS
ASSSSSPSSSGPASAPSTCSFTYPIRAGNDPGVAPGGTGGGLLYGRESAPPPTAPFNLAD
INDVSPSGGFVAELLRPELDPVYIPPQQPQPPGGGLMGKFVLKASLSAPGSEYGSPSVIS
VSKGSPDGSHPVVVAPYNGGPPRTCPKIKQEAVSSCTHLGAGPPLSNGHRPAAHDFPLGR
QLPSRTTPTLGLEEVLSSRDCHPALPLPPGFHPHPGPNYPSFLPDQMQPQVPPLHYQELM
PPGSCMPEEPKPKRGRRSWPRKRTATHTCDYAGCGKTYTKSSHLKAHLRTHTGEKPYHCD
WDGCGWKFARSDELTRHYRKHTGHRPFQCQKCDRAFSRSDHLALHMKRHF

### Designing of Nuclear Localization Sequences for Stem Cell Factors

Most stem cell inducing factors are nuclear proteins. In order to increase the effectiveness of the transducible stem cell proteins described herein, we hypothesized that fusion of a nuclear localization signal sequence to each stem cell factor may increase their nuclear localization, and hence increase the effectiveness of these proteins in reprogramming somatic cells. Because there is no KpnI/XhoI/AgeI site in mouse Oct4, mSox2, mKklf4, mMyc optimized sequences, the SV40 Large T nuclear localization sequence (NLS) was inserted into either a KpnI or XhoI site for each respective gene.

### SV40 Large T-NLS (KpnI/xhoI/KpnI)

The nuclear translocation peptide PPKKKRKV (from pJG4-5, SEQ ID NO: 73) was also optimized with E. coli codons. Because AgeI and Xho were next to each other, sequential digestions were performed.

hSox2 optimized (XhoI/R1) Noncutters AgeI and XhoI

hMyc optimized (xhoI/R1) Noncutters AgeI, KpnI and XhoI

hoct4 optimized (NcoI/R1) Noncutters NcoI (KpnI and XhoI not available)

HKlf4 optimized (xhoI/R1) Noncutters AgeI and XhoI

The following oligonucleotides were synthesized for generating nuclear targeting stem cell factors.

Age/xhoSense
(SEQ ID NO: 53)
ACCGG TCCGC CTAAA AAGAA ACGCAA AGTAC TCGAG
Age/Xho Rev-comp
(SEQ ID NO: 54)
CTCGA GTACT TTGCG TTTCT TTTTA GGCGG ACCGG T
Nls-sen (age/xho)
(SEQ ID NO: 55)
CCGGT CCGCC TAAAA AGAAA CGCAA AGTAC
Nls-anti(age/Xho)
(SEQ ID NO: 56)
TCGAG TACTT TGCGT TTCTT TTTAG GCGGA
Nls-sen (age/xho)-P
(SEQ ID NO: 57)
CCGGT CCTAA AAAGA AACGC AAAGT AC
Nls-anti(age/Xho)-P
(SEQ ID NO: 58)
TCGAG TACTT TGCGT TTCTT TTTAG GA
Nco1NLS sense:
(SEQ ID NO: 59)
CCATG GCCCC GCCTA AAAAG AAACG CAAAG TAGCC ATGG
Nco1NLS antisense
(SEQ ID NO: 60)
CCATG GCTAC TTTGC GTTTC TTTTT AGGCG GGGCC ATGG
Nco1NLS sense
(SEQ ID NO: 61)
CATGG CCCCG CCTAA AAAGA AACGC AAAGT AGC
Nco1NLS antisense
(SEQ ID NO: 62)
CATGG CTACT TTGCG TTTCT TTTTA CGCGG GGC
Nco1NLS sense-G-P
(SEQ ID NO: 63)
CATGG CCCCT AAAAA GAAAC GCAAA GTAC
Nco1NLS antisense-G-P
(SEQ ID NO: 64)
CATGG TACTT TGCGT TTCTT TTTAG GGGC

Transducible dominant negative p53, MDM2 and p53 tetramirazation domains were used as somatic cell reprogramming factors. Below are the DNA sequences for these genes, as well as primers used for cloning.

| HDM2 (accession no. Z12020) | |
|---|---|
| Primers | |
| (SEQ ID NO: 65) | |
| HDM2-F: GATCC TCGAG ATGTG CAATA CCAAC ATGTC TGTAC C | |
| (SEQ ID NO: 66) | |
| HDM2-R: GATCG AATTC CTAGG GGAAA TAAGT TAGCA CAATC | |
| (SEQ ID NO: 67) | |
| Cloned Sequence | |
| 1 | atgtgcaata ccaacatgtc tgtacctact gatggtgctg taaccacctc acagattcca |
| 61 | gcttcggaac aagagaccct ggttagacca aagccattgc ttttgaagtt attaaagtct |
| 121 | gttggtgcac aaaaagacac ttatactatg aaagaggttc ttttttatct tggccagtat |
| 181 | attatgacta aacgattata tgatgagaag caacaacata ttgtatattg ttcaaatgat |
| 241 | cttctaggag atttgtttgg cgtgccaagc ttctctgtga aagagcacag gaaaatatat |
| 301 | accatgatct acaggaactt ggtagtagtc aatcagcagg aatcatcgga ctcaggtaca |
| 361 | tctgtgagtg agaacaggtg tcaccttgaa ggtgggagtg atcaaaagga ccttgtacaa |
| 421 | gagcttcagg aagagaaacc ttcatcttca catttggttt ctagaccatc tacctcatct |
| 481 | agaaggagag caattagtga gacagaagaa aattcagatg aattatctgg tgaacgacaa |
| 541 | agaaaacgcc acaaatctga tagtatttcc ctttcctttg atgaaagcct ggctctgtgt |
| 601 | gtaataaggg agatatgttg tgaaagaagc agtagcagtg aatctacagg gacgccatcg |
| 661 | aatccggatc ttgatgctgg tgtaagtgaa cattcaggtg attggttgga tcaggattca |
| 721 | gtttcagatc agtttagtgt agaatttgaa gttgaatctc tcgactcaga agattatagc |
| 781 | cttagtgaag aaggacaaga actctcagat gaagatgatg aggtatatca agttactgtg |
| 841 | tatcaggcag gggagagtga tacagattca tttgaagaag atcctgaaat ttccttagct |
| 901 | gactattgga aatgcacttc atgcaatgaa atgaatcccc cccttccatc acattgcaac |
| 961 | agatgttggg cccttcgtga gaattggctt cctgaagata aagggaaaga taaaggggaa |
| 1021 | atctctgaga aagccaaact ggaaaactca acacaagctg aagagggctt tgatgttcct |
| 1081 | gattgtaaaa aaactatagt gaatgattcc agagagtcat gtgttgagga aaatgatgat |
| 1141 | aaaattacac aagcttcaca atcacaagaa agtgaagact attctcagcc atcaacttct |
| 1201 | agtagcatta tttatagcag ccaagaagat gtgaaagagt ttgaaaggga agaaacccaa |
| 1261 | gacaaagaag agagtgtgga atctagtttg ccccttaatg ccattgaacc ttgtgtgatt |
| 1321 | tgtcaaggtc gacctaaaaa tggttgcatt gtccatggca aaacaggaca tcttatggcc |
| 1381 | tgctttacat gtgcaaagaa gctaaagaaa aggaataagc cctgcccagt atgtagacaa |
| 1441 | ccaattcaaa tgattgtgct aacttatttc ccctag |
| p53R173h primers for amplifying p53R173H constructs | |
| (SEQ ID NO: 68) | |
| p53-F: GATCC TCGAG ATGGA GGAGC CGCAG TCAGA TCC | |
| (SEQ ID NO: 69) | |
| p53-393R: GATCG AATTC TCAGT CTGAG TCAGG CCCTT CTGTC | |
| Mouse MDM2 (accession no. X58876.1) | |
| Primers | |
| (SEQ ID NO: 70) | |
| Mdm2-F: GATCC TCGAG ATGTG CAATA CCAAC ATGTC TGTGT C | |
| (SEQ ID NO: 71) | |
| Mdm2-R: GATCG AATTC CTAGT TGAAG TAACT TAGCA CAATC | |
| (SEQ ID NO: 72) | |
| Cloned sequence | |
| 1 | atgtgcaata ccaacatgtc tgtgtctacc gagggtgctg caagcacctc acagattcca |
| 61 | gcttcggaac aagagactct ggttagacca aaaccattgc ttttgaagtt gttaaagtcc |
| 121 | gttggagcgc aaaacgacac ttacactatg aaagagatta tattttatat tggccagtat |
| 181 | attatgacta agaggttata tgacgagaag cagcagcaca ttgtgtattg ttcaaatgat |
| 241 | ctcctaggag atgtgtttgg agtcccgagt ttctctgtga aggagcacag gaaaatatat |
| 301 | gcaatgatct acagaaattt agtggctgta agtcagcaag actctggcac atcgctgagt |
| 361 | gagagcagac gtcagcctga aggtgggagt gatctgaagg atcctttgca agcgccacca |
| 421 | gaagagaaac cttcatcttc tgatttaatt tctagactgt ctacctcatc tagaaggaga |
| 481 | tccattagtg agacagaaga gaacacagat gagctacctg gggagcggca ccggaagcgc |
| 541 | cgcaggtccc tgtcctttga tccgagcctg ggtctgtgtg agctgaggga gatgtgcagc |
| 601 | ggcggcacga gcagcagtag cagcagcagc agcgagtcca cagagacgcc ctcgcatcag |
| 661 | gatcttgacg atggcgtaag tgagcattct ggtgattgcc tggatcagga ttcagtttct |
| 721 | gatcagttta gcgtggaatt tgaagttgag tctctggact cggaagatta cagcctgagt |
| 781 | gacgaagggc acgagctctc agatgaggat gatgaggtct atcgggtcac agtctatcag |
| 841 | acaggagaaa gcgatacaga ctcttttgaa ggagatcctg agatttcctt agctgactat |
| 901 | tggaagtgta cctcatgcaa tgaaatgaat cctccccttc catcacactg caaaagatgc |
| 961 | tggacccttc gtgagaactg gcttccagac gataagggga aagataaagt ggaaatctct |
| 1021 | gaaaaagcca aactggaaaa ctcagctcag gcagaagaag gcttggatgt gcctgatggc |
| 1081 | aaaaagctga cagagaatga tgctaaagag ccatgtgctg aggaggacag cgaggagaag |
| 1141 | gccgaacaga cgcccctgtc ccaggagagt gacgactatt cccaaccatc gacttccagc |
| 1201 | agcattgttt atagcagcca agaaagcgtg aaagagttga aggaggaaac gcagcacaaa |
| 1261 | gacgagagtg tggaatctag cttctccctg aatgccatcg aaccatgtgt gatctgccag |
| 1321 | gggcggccta aaaatggctg cattgttcac ggcaagactg gacacctcat gtcatgtttc |
| 1381 | acgtgtgcaa agaagctaaa aaaaagaaac aagccctgcc cagtgtgcag acagccaatc |
| 1441 | caaatgattg tgctaagtta cttcaactag |

### Preparation of Recombinant Somatic Cell Reprogramming Factor Proteins

Mouse and human reprogramming factors (Oct4, Sox2, Klf4, c-Myc), where individually inserted into a pTAT-HA vector, as described above. Additionally, SALL4, Utf1, dominant negative p53-175h, GFP, MDM2, and HDM2 and their nuclear localization versions were inserted into a pTAT-HA vector, as described above. The DNA for these factors was subsequently cloned using standard molecular biology techniques, well known to those of ordinary skill in the art. DNA sequences were confirmed by sequencing, and the recombinant clones were subsequently transformed in E. coli (BL21DE3Plys). Bacterial colonies were innoculated into TB or LB broth containing either carbenicilin or ampicillin, and grown at 37° C. until the optical density of the respective preparation reached about 0.5-1 (at 600 nm). Recombinant protein expression was then induced with IPTG. After induction with IPTG, cells were allowed to grow for an additional 2-4 hours at 37° C., or overnight at 18° C. Cells were then pelleted in a microfuge, followed by aspiration of the respective supernatant. Cell pellets were then suspended in lysis buffer (CelLyuc™ B Plus Kit, Sigma Aldrich, Mo., USA), lysed, and the lysate centrifuged. The pellet for each respective sample was resuspended in 6M Urea. The heterologous proteins were then affinity-purified with nickel agarose beads (Novagen, EMD Bioscienses, San Diego, Calif.) under denaturing conditions (6M Urea). Eluted proteins were then stored at −80° C. until used.

### Protein Handling

Immediately prior to protein use, aliquots of proteins were thawed on ice (for approximately 10 minutes). The proteins were combined with Z buffer (8 M urea, 100 mM NaCl and 20 mM HEPES, pH 8.0) in a 1:3 ratio, unless otherwise indicated, and incubated at room temperature for 10-30 minutes. The proteins were spot dialyzed on Millipore membranes (shiny side up) in 250 int PBS (at a ratio of about 1:1,000) in a beaker on ice for 30 minutes. Protein concentration was estimated by comparing Coomasie staining in SDS-PAGE gels.

### Example 1

### Reprogramming of Mouse Embryonic Fibroblasts

MEF cells (ATCC catalog no. SCRC-1008) were plated at 1.13×10⁵ cells per well on a 6-well plate (day 0) and incubated overnight in HDF medium. On day 1, the cells were treated with three purified somatic cell reprogramming factors operatively linked to the TAT peptide (m-Oct4 at 15.6 nM, m-Sox2 at 34.1 nM, at m-Klf4 22.5 nM), GFP-TAT fusion protein, VPA (2 mM) and sodium azide (0.002%). Three days later (day 4), the media was replaced and cells were treated with the same components at the same concentration. The cells were then harvested from the dishes on day 7, and frozen at −80° C. Upon thawing on day 8, the cells were put onto mitomycin-treated MEF cells (feeder) in ES cell culture basal medium (“ES-cm,” containing 10% FBS in DMEM/F12 supplemented with 2 mM glutamine, 1×MEM NEAA, 100 μM 2-mercaptoethanol, 4 μg/mL β-FGF, 100 U/mL penicillin, and 100 μg/mL streptomycin). The medium in each plate was changed daily.

On day 20, stem cell like colonies were harvested and treated with 300 μL of collagenase IV solution in individual wells of a 96-well plate, for 3˜5 minutes. The cell suspensions were transferred onto the new MEF-MITC feeder cells in ES-cm medium.

On day 23, one of the stem cell like colonies was stained AP-positive.

### Example 2

### Reprogramming Human Dermal Fibroblasts with Three Purified Somatic Cell Reprogramming Factors

### MEF-MITC Feeder Cell Preparation

0.1% gelatin solution was added to the wells of a 6-well cell culture plate and incubated for 45 minutes in a 37° C./5% CO₂ incubator. Gelatin solution was removed prior to plating cells. MEF-MITC were plated at 1.6×10⁵ cell per well on gelatin coated 6 well plates. The feeder cells were allowed to grow for 24 hours in a 37° C./5% CO₂ incubator.

HDFn cells were plated at 2.11×10⁵ cells per well on a 6 well plate and incubated overnight. Three purified somatic cell reprogramming factors (each operatively linked to the TAT peptide) (m-Oct4 at 15.6 nM, m-Sox2 34.1 nM, m-Klf4 22.5 nM), GFP-TAT chimera, VPA (2 mM) and SA (0.002%) were added on day 1, day 2 and day 3, in HDF-culture medium. Before addition of the compounds on each day, the medium in each well was changed. On Day 9, cells were transferred to MEF-MITC feeder cells.

On day 9, treated HDFn cells were dissociated via Collagenase IV and replated to feeder cells (MEF-MITC). For passaging, treated HDFn cells were incubated with PBS containing 1 mg/mL Collagenase TV (Invitrogen) at 37° C. After approximately 5 minutes, collagenase was removed, and 2 mL ES-cm media was added to the wells. Cells were collected into a 15 mL conical tube. The tube was centrifuged for 5 minutes at 800 RPM at 4° C.

The cell pellet was resuspended in an appropriate volume of medium of ES-cm medium (described above) and transferred to three wells of feeder cells (preparation described above). The split ratio was 1:3. This stage was defined as passage 1. Potential human induced pluripotent stem cells (h-IPSCs) were grown on MEF-MITC, with embryonic stem cell culture medium (ES-cm) changed daily. Five days after transferring the treated cells to feeder cells, the cells looked morphologically similar to embryonic stem cells (14 days after exposure to somatic cell reprogramming factors). One colony was stained AP positive on Day 34 using Millipore AP staining kit (catalog no. SCR.004).

### Example 3

### Reprogramming of Human Dermal Fibroblasts with Three Purified Somatic Cell Reprogramming, Factors, Valproic Acid, Sodium Azide and Vitamin C

Human dermal fibroblast (HDF) cells were plated in HDF medium, at 4×10⁵ cells per well in a 6-well plate (day 0). The cultures were incubated overnight in a 37° C./5% CO₂ incubator. The HDF medium was then replaced, and the cell cultures were treated with purified somatic cell reprogramming factors m-Sox2 (34.1 nM), m-Klf4 (22.5 nM), m-Oct4 (15.6 nM), as well as 2 mM valproic acid, sodium azide (0.002%), and vitamin C (10 μM) (day 1). The cultures were incubated for 24 hours in a 37° C./5% CO₂ incubator. The next day (day 2), the medium was replaced with fresh HDF medium. Cells were then treated as on day 1. The cultures were again incubated overnight in a 37° C./5% CO₂ incubator.

On day 3, the HDF medium was replaced with HEScGRO Basal Medium for Human ES Cell Culture from Millipore (presently Millipore catalog no. SCM020-100). Cells were treated with m-Sox2, m-Oct4 and m-Klf4 tranducible proteins, valproic acid and sodium azide, at the same concentrations as days 1 and 2. The cultures were again incubated overnight in a 37° C./5% CO₂ incubator.

On day 4, the Hescgro medium was replaced with fresh HEScGRO Basal Medium for Human ES Cell Culture from Millipore and the cells were treated with the same components given on day 3. Cultures were incubated overnight in a 37° C./5% CO, incubator.

On days 5, the HEScGRO Basal Medium for Human ES Cell Culture from Millipore was again replaced with fresh Hescgro medium, and the cells were treated with the same components given on day 3. Cultures were incubated overnight in a 37° C./5% CO₂ incubator.

On Day 6, medium in each well was replaced with fresh Hescgro medium. Cells were then treated with valproic acid, sodium azide and vitamin C, at the concentrations used on days 1 and 2. Cell cultures were then incubated for 3 days in a 37° C./5% CO₂ incubator.

On day 8, feeder cells, HFF-1 (ATCC presently catalog no. SCRC-1041) were plated in sterile cell culture dishes. These cells had previously been treated with mitomycin C and frozen in liquid nitrogen until needed. Prior to plating the feeder cells, 0.1% gelatin solution was added to the wells and incubated for 45 minutes in a 37° C./5% CO₂ incubator. Gelatin solution was removed and the HFF-MITC cells were plated at a density of 1.6×10⁵ cell per well on the gelatin coated 6 well plates. The feeder cells were allowed to grow for 24 hours in a 37° C./5% CO₂ incubator.

On day 9, treated HDF cells were dissociated by replacing the medium with 1×PBS containing 1 mg/mL collagenase IV (Invitrogen, Carlsbad, Calif.). Cells were then incubated for approximately 5 minutes at 37° C. (cells had detached at this point). Next, 2 mL Hescgro media was added to each well containing a PBS cell suspension. Cells suspensions were then collected and consolidated into a 15 mL conical tube and centrifuged for 5 minutes, at 800 RPM and 4° C. The cell pellet was then resuspended in an appropriate volume of Hescgro medium, and transferred to three wells of feeder cells. The split ratio was routinely 1:3 (HDF:feeder). This transfer was defined as passage 1. Potential human iPSCs were grown on the HFF-MITC feeder cells. Hescgro medium was changed daily. Stem cell-like colonies appeared on Day 15.

### Example 4

### Reprogramming of Human Dermal Fibroblasts with 5 Purified Somatic Cell Reprogramming Factors, Valproic Acid, Sodium Azide and Vitamin C

HDF adult cells were plated at 4×10⁵ cells per well in a 6 well plate and incubated overnight in HDF medium, in a 37° C./5% CO₂ incubator. The next day, cells were treated purified somatic cell reprogramming factors (1) m-Sox2 (34.1 nM), (2) m-Klf4 (22.5 nM), (3) m-Oct4 (15.6 nM), (4) m-Myc (11.5 nM), (5) p53-r175h (5.2 nM), as well as 2 mM valproic acid, sodium azide (0.002%) and 10 μM vitamin C. Cell cultures were then incubated for 24 hours in a 37° C./5% CO₂ incubator.

The next day (day 2), cell cultures were treated as indicated above for day 1 (medium was not replaced). The cultures were then incubated for 24 hours in a 37° C./5% CO₂ incubator.

On day 3, the medium in each well was changed to Hescgro medium, Cell cultures underwent the same treatment as indicated for days 1 and 2, except no vitamin C was added to the cultures. The cultures were then incubated for 24 hours in a 37° C./5% CO₂ incubator. The medium was again changed on day 4 to fresh Hescgro medium. The cultures were then treated in an identical manner to that of the treatment on day 3. Cell cultures were incubated for 24 hours in a 37° C./5% CO₂ incubator.

The medium was again changed on day 5 to flesh Hescgro medium (Millipore presently catalog no. SCM020-100). The cultures were then treated in an identical manner to that of the treatment on day 3. Cell cultures were incubated for 24 hours in a 37° C./5% CO₂ incubator.

On Day 6, Hescgro medium was replaced with fresh Hescgro medium. Cells were then treated with valproic acid, sodium azide and vitamin C at the concentrations indicated for day 1.

On day 8, feeder cells, HFF-1 (ATCC catalog no. SCRC-1041) were plated in sterile cell culture dishes. These cells had previously been treated with mitomycin-C (HFF-MITC) and frozen in liquid nitrogen until needed. Prior to plating the feeder cells, 0.1% gelatin solution was added to the wells and incubated for 45 minutes in a 37° C./5% CO₂ incubator. Gelatin solution was removed and the HFF-MITC cells were plated at a density of 1.6×10⁵ cell per well on the gelatin coated 6 well plates. The feeder cells were allowed to grow for 24 hours in a 37° C./5% CO₂ incubator.

On day 9, treated HDF cells were dissociated by replacing the medium with 1×PBS containing 1 mg/mL collagenase IV (Invitrogen). Cells were then incubated for approximately 5 minutes at 37° C. (cells had detached at this point). Next, 2 mL Hescgro media was added to each well containing a PBS cell suspension. Cells suspensions were then collected and consolidated into a 15 mL conical tube and centrifuged for 5 minutes, at 800 RPM and 4° C. The cell pellet was then resuspended in an appropriate volume of Hescgro medium, and transferred to three wells of feeder cells. The split ratio was 1:2 (HDF cells:feeder cells). This transfer was defined as passage 1. Potential human iPSCs were grown on the HFF-MITC feeder cells. Hescgro medium was changed daily. Stem cell-like colonies appeared on Day 11.

### Example 5

### Reprogramming of Human Dermal Fibroblasts and MEF with Somatic Cell Reprogramming Factors, Valproic Acid and Sodium Azide

The day before introducing proteins into cells, HDFn cells or MEF cells, or MEF-Oct4-GFP cells were thawed and plated as 2.0×10⁵ cells/well in multiple 6-well plates (defined as day 0). Fibroblast growth medium (Cell Applications, Inc, CA, USA) was used for culture. Cultures were incubated for 24 hours in a 37° C./5% CO₂ incubator.

On day 1, purified somatic cell reprogramming factors were added to each well at the following amounts—m-KLF4 at 1.5 μg/well, m-SOX2 at 3 μg/well, m-Oct4 at 1.5 μg/well, m-Myc at 1.5 μg/well, and m-p53-175 h at 1.5 μg/well. As described above, each protein was diluted with Buffer Z (8 M urea, 100 mM NaCl and 20 mM HEPES, pH 8.0) at a dilution ratio of 1:3 protein(volume):Buffer Z(volume), prior to introduction into the cell culture wells. Proteins were then incubated for 30 minutes at room temperature. Z-buffer-treated proteins were then dialyzed against ice cold PBS on the MF-Membrane Filters (Millopore, Mass., USA) for 30 minutes.

Before purified transducible somatic cell reprogramming factors were added into fibroblast cell culture, cell culture medium was changed. Also, valproic acid (2 mM) and sodium azide (0.002% final concentration in medium) were added into each respective well, together with the 5 somatic cell reprogramming factors proteins. Cells were then incubated for 48 hours in a 37° C./5% CO₂ incubator.

On day 3, the cell culture medium was replaced with fresh fibroblast growth medium. Each cell culture was individually treated with the 5 transducible proteins, valproic acid and sodium azide. Cells were then incubated for 48 hours in a 37° C./5% CO₂ incubator. On day 5, the same process (as day 3) was repeated. On day 7, the same process (as on days 3 and 5) was repeated.

On Day 8, feeder cells (HFF-MITC or MEF-MITC) were thawed to 6-well plates and cultured with 15% PBS containing DMEM. Cells were then incubated overnight in a 37° C./5% CO₂ incubator.

On Day 9, HDF or MEF-Oct4 cells were transferred onto feeder cells and incubated with either HEScGRO Basal Medium (Millipore, Billerica, Mass.) for HDF cells, or ES-cm media (DMEM/F12 with 20% PBS. 1% NEAA, 2 mM Glutamine, 1% P/S, 0.0008% BME, and 4 μg/mL bFGF) for MEF-Oct4 cells. Optical microscopy images indicated stem cell like colonies started to appear on Day 12. Additionally, fluorescence microscopy images showed that MEF-Oct4-GFP cell clusters appeared on Day 12.

On Day 22, all cells in each well were treated with collagenase IV and collected by centrifuge. Collected cells were re-suspended in 100 μL PBS and subcutaneously injected into the right flanks of Nu/Nu mice.

### Example 6

### Reprogramming of Human Dermal Fibroblasts with Three Transducible Somatic Cell Reprogramming Factors

On day 0, HDFn cells, at passage 3, were seeded in HDF medium (Cell Applications) in each well of a 6-well plate. On day 1, HDF cells were at 90% confluence, and medium was changed to fresh IMF media containing 2 mM sodium valproic acid and 0.002% sodium azide.

Each somatic cell reprogramming factor protein solution was mixed with Z-buffer at the 1:3 ratio and incubated for 10 minutes at room temperature, and then dialyzed using the ME-Membrane Filter (Millipore) on cold PBS for 30 minutes.

After dialysis, three protein solutions were directly added to each well at the final concentrations of 34.1 nM for m-Sox2, 22.5 nM for m-Klf4, and 15.6 nM for m-Oct4, and incubated for 48 hours in a 37° C./5% CO₂ incubator. This protein treatment and incubation period was carried out a total four times (day 1, day 3, day 5, and day 7). Prior to protein treatment on each day, the medium in each well was replaced with fresh medium.

On day 9, the treated HDF cells were trypsinyzed and transferred onto the MEF-MTIC feeder cells (ATCC) in ES-cm medium (10% FBS in DMEM/F12 supplemented with 2 mM glutamine, 1×MEM NEAA, 100 μM 2-mercaptoethanol, 4 mg/mL β-FGF, 100 U/mL penicillin, and 100 μg/mL streptomycin). After day 9, ES-cm media was changed daily.

Five stem cell-like colonies appeared on Day 12 and an additional five colonies appeared on Day 16. On day 22, the HDF cells were again transferred onto the new feeder cells by collagenase IV treatment. Alkaline phosphatase staining was conducted on day 31 using an alkaline phospatase (AP) detection kit (Millipore). One colony was stained AP positive.

### Example 7

### Reprogramming of Human Dermal Fibroblasts with Three Transducible Somatic Cell Reprogramming Factors

On day 0, HDFa cells, at passage 3, were seeded in HDF medium (Cell Applications) in each well of a 6-well plate. On day 1, HDF cells were at about 30% confluence, and medium was changed to fresh Hescgro medium containing 2 mM sodium valproic acid (VPA), 0.002% sodium azide (SA) and 10 μM vitamin C (VC).

Each somatic cell reprogramming factor protein solution was mixed with Z-buffer at the 1:3 ratio and incubated for 10 minutes at room temperature, and then dialyzed with cold PBS for 30 minutes.

After dialysis, three protein solutions were directly added to each well at the final concentrations of 34.1 nM for Sox2, 22.5 nM for Klf4, and 15.6 nM for Oct4, and incubated for 24 hours in a 37° C./5% CO₂ incubator. This protein treatment and incubation period was carried out a total five times (day 1, day 2, day 3, day 4 and day 5). Prior to protein treatment on each day, the medium in each well was replaced with fresh Hescgro medium. Further medium change containing VPA, SA and VC was done on day 7 and day 9.

On day 11, the treated HDF cells were transferred onto the HFF-MITC feeder cells (ATCC) in Hescgro medium by collagenase IV treatment. After day 11, ES-cm media was changed daily.

Multiple stem cell-like colonies appeared on Day 14. On day 17, alkaline phosphatase staining was conducted using an alkaline phospatase detection kit. Multiple colonies (at least 50 colonies) were stained AP positive.

### Example 8

### Reprogramming of Human Dermal Fibroblasts with Purified Somatic Cell Reprogramming Factors, Valproic Acid and Sodium Azide

The day before introducing proteins into cells, HDFa cells were thawed and plated at the density of 2.0×10⁵ cells per well in multiple 6-well plates (defined as day 0). Fibroblast growth medium (Cell Applications, mc, CA, USA) was used for culture. Cultures were incubated for 24 hours in a 37° C./5% CO₂ incubator.

On Day 1, purified somatic cell reprogramming factors were added to each well at the following amounts—m-KLF4 at 1.5 μg/well, m-SOX2 at 3 μg/well, m-Oct4 at 1.5 μg/well, m-Myc at 1.5 μg/well, and p53-175h at 1.5 μg/well (each protein had a TAT domain). As described above, each protein was diluted with Buffer Z (8 M urea, 100 mM NaCl and 20 mM HEPES, pH 8.0) at a dilution ratio of 1:3 protein:Buffer Z, prior to introduction into the cell culture wells. Proteins were then incubated for 30 minutes at room temperature. Z-buffer-treated proteins were then dialyzed against ice cold PBS for 30 minutes.

Before purified transducible somatic cell reprogramming factors were added into fibroblast cell culture, cell culture medium was changed. Also, valproic acid (2 mM) and sodium azide (0.002% final concentration in medium) were added into each respective well, together with the 5 somatic cell reprogramming factors proteins. Cells were then incubated for 48 hours in a 37° C./5% CO₂ incubator.

On day 2, the cell culture medium was replaced with fresh fibroblast growth medium. On day 3, each cell culture was individually treated with the 5 transducible proteins, valproic acid and sodium azide. Cells were then incubated in a 37° C./5% CO₂ incubator and culture medium was replaced with fresh fibroblast growth medium on day 4. On day 5, the same process of protein treatment (as day 3) was repeated. On day 7, the same process (as on days 3 and 5) was repeated. On day 6 and 8, culture medium was replaced with fresh fibroblast growth medium.

On Day 8, feeder cells (HFF-MITC) were thawed to 6-well plates and cultured with 15% FBS containing DMEM. Cells were then incubated overnight in a 37° C./5% CO₂ incubator.

On Day 9, HDFa cells were transferred onto feeder cells and incubated with HEScGRO Basal Medium (Millipore, Billerica, Mass.), Culture medium was replaced daily. Optical microscopy images indicated stem cell like colonies started to appear on Day 12, and over 50 colonies of pluripotent stern-cell like cells were observed under microscopy.

### Example 9

### Reprogramming of Mouse Embryonic Fibroblasts with Three Purified Somatic Reprogramming Proteins (without VPA, Sodium Azide, or Vitamin C)

MEF cells (ATCC currently catalog no. SCRC-1008) were plated at 1.13×10⁵ cells per well on a 6-well plate (day 0) and incubated overnight in 15% FBS/DMEM medium.

Each somatic cell reprogramming factor solution was mixed with Z-buffer at the 1:4 ratio and incubated for 10 minutes at room temperature, and then dialyzed with cold PBS for 30 minutes.

On day 1, day 2, day 3 and day 4, three purified somatic cell reprogramming factors operatively linked to the TAT peptide (m-Oct4 at 11.2 nM, m-Sox2 at 24.6 nM, at m-Klf4 16.2 nM) as well as GFP-TAT fusion protein were directly added to each well in a 6-well plate. Before purified transducible somatic cell reprogramming factors were added, the cell culture medium was changed to a fresh one.

On day 5, cells were transferred to MEF-MITC feeder cells in ES-cm media by collagenase IV treatment. The media was changed to fresh ES-cm media on day 6, and at least one stem cell like colony was observed on day 7.

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method for reprogramming at least one somatic cell, comprising:
growing a somatic cell culture;
treating the somatic cell culture with a solution comprising:
(a) an effective amount of at least one purified somatic cell reprogramming factor; and
(b) an effective amount of at least one somatic cell reprogramming enhancing factor,
harvesting the treated somatic cell culture to form a treated somatic cell suspension;
plating the treated somatic cell suspension to form a cell culture; and
growing the cell culture until at least one somatic cell has been reprogrammed.

2. The method of claim 1, wherein the reprogrammed somatic cell is an induced pluripotent stem cell.

3.-5. (canceled)

6. The method of claim 1, wherein the cell is a human cell.

7. The method of claim 1, wherein the at least one somatic cell reprogramming enhancing factor is a histone deacetylase inhibitor.

8. The method of claim 7, wherein the histone deacetylase inhibitor is selected from the group consisting of valproic acid (VPA), suberoylanilide hydroxamic acid (SAHA) and trichostatin A (TSA).

9. The method of claim 1, wherein the at least one somatic cell reprogramming factor is selected from the group consisting of Oct4, Sox2, Klf4, c-Myc, Utf1, Lin28, Sall4, AID, MDM2/HDM2, dominant negative p53, p53 inhibitors, and their protein family members.

10.-14. (canceled)

15. The method of claim 1, wherein the one or more purified somatic cell reprogramming factors is delivered to the cell by liposome, electroporation, or chemical transduction.

16. The method of claim 1, wherein the at least one purified somatic cell reprogramming factor is operatively linked to a protein transduction domain.

17. The method of claim 6, wherein the human cell is a fibroblast.

18. The method of claim 16, wherein the protein transduction domain is HIV-TAT.

19. (canceled)

20. The method of claim 1, wherein the at least one somatic cell reprogramming enhancing factor is a factor which which mimic hypoxia, glycolysis upregulating conditions or respiration inhibiting conditions.

21. (canceled)

22. The method of claim 1, wherein the at least one somatic cell reprogramming enhancing factor is vitamin C.

23. The method of claim 1, comprising at least two somatic cell reprogramming enhancing factors.

24. The method of claim 23, wherein the at least two somatic cell reprogramming factors are VPA and sodium azide.

25. The method of claim 1, comprising at least three somatic cell reprogramming enhancing factors.

26. The method of claim 25, wherein the at least three somatic cell reprogramming factors are VPA, sodium azide and vitamin C.

27.-28. (canceled)

29. A method for generating at least one induced pluripotent stem (iPS) cell comprising:
growing a somatic cell culture;
treating the somatic cell culture with a solution comprising:
(a) an effective amount of at least one chimeric protein comprising a protein transduction domain operatively linked to a somatic cell reprogramming factor; and
(b) an effective amount of at least one somatic cell reprogramming enhancing factor;
harvesting the treated somatic cell culture to form a treated somatic cell suspension;
plating the treated somatic cell suspension to form a cell culture; and
growing the cell culture until at least one induced pluripotent stem cell is generated.

30. The method of claim 29, wherein the at least one somatic cell reprogramming enhancing factor is a histone deacetylase inhibitor.

31. The method of claim 30 wherein the histone deacetylase inhibitor is selected from valproic acid (VPA), suberoylanilide hydroxamic acid (SAHA) and trichostatin A (TSA).

32.-34. (canceled)

35. The method of claim 29 wherein the at least one somatic cell reprogramming factor is selected from Oct4, Sox2, Klf4, c-Myc, Utf1, Lin28, Sa114, AID, MDM2/HDM2, dominant negative p53, p53 inhibitors and their protein family members.

36.-41. (canceled)

42. The method of claim 29, wherein the protein transduction domain is HIV-TAT.

43. (canceled)

44. The method of claim 29, wherein the at least one somatic cell reprogramming enhancing factor is a factor which mimic hypoxia, glycolysis upregulating conditions or respiration inhibiting conditions.

45. The method of claim 29, wherein somatic cell is a fibroblast.

46. The method of claim 29, wherein the at least one somatic cell reprogramming enhancing factor vitamin C.

47. The method of claim 29, comprising at least two somatic cell reprogramming enhancing factors.

48. The method of claim 47, wherein the at least two somatic cell reprogramming factors are VPA and sodium azide.

49. The method of claim 29, comprising at least three somatic cell reprogramming enhancing factors.

50. The method of claim 49, wherein the at least three somatic cell reprogramming factors are VPA, sodium azide and vitamin C.

51.-53. (canceled)

54. A method for generating induced pluripotent stem (iPS) cells comprising:
growing a somatic cell culture;
treating the somatic cell culture with a solution comprising:
(a) an effective amount of a chimeric protein comprising purified Sox2 N-terminally linked to HIV-TAT;
(b) an effective amount of a chimeric protein comprising purified Klf4 N-terminally linked to HIV-TAT;
(c) an effective amount of a chimeric protein comprising purified Oct4 N-terminally linked to HIV-TAT;
(d) an effective amount of valproic acid; and
(e) an effective amount of sodium azide;
harvesting the treated somatic cell culture to form a treated somatic cell suspension;
plating the treated somatic cell suspension on a layer or partial layer of feeder cells to form a cell culture; and
growing the cell culture until at least one induced pluripotent stem cell is generated.

55. The method of claim 54, wherein the solution further comprises an effective amount of a chimeric protein comprising purified c-Myc N-terminally fused to HIV-Tat or a fusion protein comprising purified dominant negative pS3 N-terminally fused to HIV-Tat.

56. The method of claim 54, wherein the solution further comprises an effective amount of vitamin C.

57.-58. (canceled)

59. The method of claim 54, wherein the somatic cell is a mammalian fibroblast.

60.-78. (canceled)
